(19) United States
(12) Patent Application Publication
(10) Pub. No.: US 2007/0122817 A1
(43) Pub. Date: May 31, 2007

(54) Methods for assembly of high fidelity synthetic polynucleotides

(76) Inventors: George Church, Brookline, MA (US); Joseph Jacobson, Newton, MA (US); Brian M. Baynes, Somerville, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/067,812

(22) Filed: Feb. 28, 2005

(51) Int. Cl. C12Q 1/68; C12P 19/34

(52) U.S. Cl. 435/6; 435/91.2

(57) ABSTRACT

Disclosed are methods of manufacturing synthetic DNAs, that is, DNAs made at least in significant part by chemical synthesis of polynucleotide polymers. Also provided are methods for assembling plural DNAs in the same pool by multiplexed assembly of synthetic oligonucleotides. In exemplary embodiments, the methods involve pre-amplification of one or more oligonucleotides using “universal” primers, reduction of the error rate in oligonucleotide and/or polynucleotide products, and sequence optimization and oligonucleotides design.

### BACKGROUND

Using the techniques of recombinant DNA chemistry, it is now common for DNA sequences to be replicated and amplified from nature and for those sequences to then be disassembled into component parts which are then recombined or reassembled into new DNA sequences. However, reliance on naturally available sequences significantly limits the possibilities that may be explored by researchers. While it is now possible for short DNA sequences to be directly synthesized from individual nucleosides, it has been generally impractical to directly construct large segments or assemblies of DNA sequences larger than about 400 base pairs. As a consequence, larger segments of DNA are generally constructed from component parts and segments which can be purchased, cloned or synthesized individually and then assembled into the DNA molecule desired.

Current methods for generating even basic oligonucleotides are expensive (e.g., US $0.11 per nucleotide) and have very high levels of errors (deletions at a rate of 1 in 100 bases and mismatches and insertions at about 1 in 400 bases). As a result, gene or genome synthesis from oligonucleotides is both expensive and prone to error. Correcting errors by clone sequencing and mutagenesis methods further increases the amount of labour and total cost (to at least US $2 per base pair). In principle, the cost of oligonucleotide synthesis can be reduced by performing massively parallel custom syntheses on microchips (Zhou et al. (2004) Nucleic Acids Res. 32:5409; Fodor et al. (1991) Science 251:767). This can now be achieved using a variety of methods, including ink-jet printing with standard reagents (Agilent; see e.g., U.S. Pat. No. 6,323,043), photolabile 5′ protecting groups (Nimblegen/Affymetrix; see e.g., U.S. Pat. No. 5,405,783; and PCT Publication Nos. WO 03/065038; 03/064699; WO 03/064026; 02/04597), photo-generated acid deprotection (Atactic/Xeotron; see e.g., X. Gao et al., Nucleic Acids Res. 29: 4744-50 (2001); X. Gao et al., J. Am. Chem. Soc. 120: 12698-12699 (1998); O. Srivannavit et al., Sensors and Actuators A. 116: 150-160 (2004); and U.S. Pat. No. 6,426,184) and electrolytic acid/base arrays (Oxamer/Combimatrix; see e.g., U.S. Patent Publication No. 2003/0054344; U.S. Pat. Nos. 6,093,302; 6,444,111; 6,280,595). However, current microchips have very low surface areas and hence only small amounts of oligonucleotides can be produced. When released into solution, the oligonucleotides are present at picomolar or lower concentrations per sequence, concentrations that are insufficiently high to drive bimolecular priming reactions efficiently.

The manufacture of accurate DNA constructs is severely impacted by error rates inherent in chemical synthesis techniques. By way of example, the table in FIG. 1 illustrates the effects of error rates on polynucleotide fidelity. For example, synthesis of a DNA having an open reading frame of 3000 base pairs using a method with an error rate of 1 base in 1000, will result in less than 5% of the copies of the synthesized DNA having the correct sequence.

A state of the art oligonucleotide synthesizer exploiting phosphoramidite chemistry makes errors at a rate of approximately one base in 200. DNAs synthesized on chips using photo labile synthesis techniques reportedly have an error rate of about 1/50, and potentially may be improved to about 1/100. High fidelity PCR has an error rate of about 1/10⁵. Even at such high fidelity duplication, for a gene 3000 bp in length, a polymerases operating ex vivo produce copies that contain an error about 3% of the time. Because the current best commercial DNA synthesis protocols represent the pinnacle of several decades of development, it seems unlikely that order of magnitude additional improvements in chemical synthesis of polynucleotides will be forthcoming in the near future.

The widespread use of gene and genome synthesis technology is hampered by limitations such as high cost and high error rate, and lack of automation. It is therefore an object of this invention to provide practical, economical methods of synthesizing custom polynucleotides, and large genetic systems. It is a further object to provide a method of producing synthetic polynucleotides that have lower error rates than synthetic polynucleotides made by methods known in the art.

### SUMMARY

Provided herein are methods that enable cost-effective production of useful, high fidelity synthetic DNA constructs by providing a group of improvements to the DNA assembly methods of Mullis (Mullis et al. (1986) Cold Spring Harb. Symp. Quant. Biol. 51 Pt 1:263) and Stemmer (Stemmer et al. (1995) Gene 164:49) which may be used individually or together. The improvements include advances in computational design of the oligonucleotides used for assembly, i.e., in the design of the “construction oligonucleotides” and for purification, i.e., the “selection oligonucleotides,” multiplexing of construction oligonucleotide assembly, i.e., making plural different assemblies in the same pool, construction oligonucleotide amplification techniques, and construction oligonucleotide error reduction techniques.

Described herein are methods for preparing a polynucleotide construct having a predefined sequence involving amplification of the oligonucleotides at various stages. The method comprises providing a pool of construction oligonucleotides having (i) partially overlapping sequences that define the sequence of the polynucleotide construct, (ii) at least one pair of primer hybridization sites flanking at least a portion of said construction oligonucleotides and common to at least a subset of said construction oligonucleotides, and (iii) cleavage sites between the primer hybridization sites and the construction oligonucleotides. The pool of construction oligonucleotides may then be amplified using at least one primer that binds to the primer hybridization sites. Optionally, the primer hybridization sites may then be removed from the construction oligonucleotides at the cleavage sites (e.g., using a restriction endonuclease, chemical cleavage, etc.). After amplification, the construction oligonucleotides may be subjected to assembly, e.g., by denaturing the oligonucleotides to separate the complementary strands and then exposing the pool of construction oligonucleotides to hybridization conditions and ligation and/or chain extension conditions.

Also described herein are methods for preparing a purified pool of construction oligonucleotides. The methods comprise contacting a pool of construction oligonucleotides with a pool of selection oligonucleotides under hybridization conditions to form duplexes. The reaction will form both stable duplexes (e.g., duplexes comprising a copy of a construction oligonucleotide and a copy of a selection oligonucleotide that do not contain a mismatch in the complementary region) and unstable duplexes (e.g., duplexes comprising a copy of a construction oligonucleotide and a copy of a selection oligonucleotide that contain one or more mismatches, e.g., base mismatches, insertions, or deletion, in the complementary region). The copies of the construction oligonucleotides that formed unstable duplexes may then be removed from the pool (e.g., using a separation technique such as a column) to form a pool of purified construction oligonucleotides. Optionally, the purification process (e.g., mixture of the construction and selection oligonucleotides) may be repeated at least once before use of the construction oligonucleotides. Additionally, the pool of construction oligonucleotides may be amplified before and/or after the various rounds of purification by selection. After forming the pool of purified construction oligonucleotides, they pool may be subjected to assembly conditions. For example, the pool of construction oligonucleotides may be exposed to hybridization conditions and ligation and/or chain extension conditions. In a variation of this purification method, the duplexes comprising construction and selection oligonucleotides may be contacted with a mismatch binding agent and the bound duplexes (e.g., duplexes containing one or more mismatches) may be removed from the pool (e.g., using a column or gel).

Also described herein are methods for preparing a plurality of polynucleotide constructs having different predefined sequences in a single pool. The method comprises (i) providing a pool of construction oligonucleotides comprising partially overlapping sequences that define the sequence of each of said plurality of polynucleotide constructs and (ii) incubating said pool of construction oligonucleotides under hybridization conditions and ligation and/or chain extension conditions. Optionally, the oligonucleotides and/or polynucleotide constructs may be subjected to one or more rounds of amplification and/or error reduction as desired. Additionally, the polynucleotide constructs may be subject to further rounds of assembly to produce even longer polynucleotide constructs. At least about 2, 4, 5, 10, 50, 100, 1,000 or more polynucleotide constructs may be assembled in a single pool.

Also described herein are methods for designing construction and/or selection oligonucleotides as well as an assembly strategy for producing one or more polynucleotide constructs. The method may comprise, for example, (i) computationally dividing the sequence of each polynucleotide construct into partially overlapping sequence segments; (ii) synthesizing construction oligonucleotides comprising sequences corresponding to the sets of partially overlapping sequence segments; and (iii) incubating said construction oligonucleotides under hybridization conditions and ligation and/or chain extension conditions. Optionally, the method may further comprise (i) computationally adding to the termini of at least a portion of said construction oligonucleotides one or more pairs of primer hybridization sites common to at least a subset of said construction oligonucleotides and defining cleavage sites between the primer hybridization sites and the construction oligonucleotides; (ii) amplifying said construction oligonucleotides using at least one primer that binds to said primer hybridization sites; and (iii) removing said primer hybridization sites from said construction oligonucleotides at said cleavage sites. Preferably such primer sites may be common to at least a portion of the construction oligonucleotides in the pool. The method may further comprise computationally designing at least one pool of selection oligonucleotides comprising sequences that are complementary to at least portions of said construction oligonucleotides, synthesizing said selection oligonucleotides, and conduction an error filtration process by hybridization the pool of construction oligonucleotides to the pool of selection oligonucleotides.

In one aspect, the invention provides a composition comprising a plurality of copies of a synthetic polynucleotide having a predefined sequence wherein said polynucleotide has a length of at least about 5, 10, or 100 kilobases, or more, and wherein at least about 1%, 5%, 10%, 20%, 50%, or more, of said copies do not contain an error is said predefined sequence. In an exemplary embodiment, the composition may be essentially free of one or more cellular contaminants without using a purifications step to remove the contaminant (e.g., the polynucleotide construct has been synthesized in a cell free manner). Cellular contaminants include those things which typically contaminant a preparation of a DNA or RNA that has been isolated from a cell or cell lysate sample, such as, for example, various proteins, lipids, lipopolysaccharides, carbohydrates, pyrogens, small molecules, etc.

In yet another aspect, the invention provides a method for synthesizing a polynucleotide construct that involves multiple rounds of amplification, error reduction, and/or assembly. For example, the method comprises: (i) providing a pool of construction oligonucleotides; (ii) amplifying the construction oligonucleotides and/or subjecting the construction oligonucleotides to one or more error reduction process; (iii) assembling the construction oligonucleotides (e.g., by exposing them to hybridization and chain extension and/or ligation conditions) to form subassemblies; (iv) amplifying the subassemblies and/or subjecting the subassemblies to one or more error reduction processes; and (v) assembling the subassemblies to form polynucleotide constructs (e.g., by exposing the subassemblies to hybridization and chain extension and/or ligation conditions). The polynucleotide constructs may then optionally be subjected to one or more rounds of amplification and/or error reduction. In various embodiments, the oligonucleotides, subassemblies, and/or polynucleotide constructs may be subjected to multiple rounds of amplification and/or error correction at each stage of assembly. The error reduction processes at any stage of assembly may include, for example, error filtration processes, error neutralization processes, and/or error correction processes. In an exemplary embodiment, shorter oligonucleotides are subjected to an error filtration process using hybridization to selection oligonucleotides, intermediate length subassemblies and/or polynucleotide constructs may be subjected to an error filtration process (e.g., by binding to a mismatch binding agent) or an error neutralization process, and long polynucleotide constructs may be subjected to an error filtration process or an error correction process.

In yet another aspect, the invention provides an iterative method for synthesizing long polynucleotide constructs. For example, the method may comprise: (i) providing a pool of input oligonucleotides under hybridization conditions and ligation and/or chain extension conditions to form at least one product polynucleotide that is longer than said oligonucleotides; (ii) amplifying said product polynucleotide(s) and/or subjecting the product polynucleotide(s) to an error reduction process; and (iii) repeating (i) and (ii) at least two times wherein said product polynucleotides constitute the input oligonucleotides in the next cycle.

In yet another aspect, the invention provides a method for multiplex assembly, in a single pool, of a plurality of polynucleotide constructs having different predefined sequences and at least one region of internal homology. For example, the method may comprise (i) providing a pool of construction oligonucleotides comprising partially overlapping sequences that define the sequence of each of said plurality of polynucleotide constructs; and (ii) exposing the pool of construction oligonucleotides to hybridization conditions and ligation and/or chain extension conditions. In certain embodiments, the oligonucleotides and/or polynucleotide constructs may be subjected to one or more rounds of amplification and/or error reduction. In an exemplary embodiment, at least about 2, 5, 10, 100, 1,000, 10,000 or more polynucleotide constructs having different predefined sequences and at least one region of internal homology may be synthesized in a single pool. For example, such methods may be useful for preparing a library of polynucleotide constructs that encode a plurality of RNAs or polypeptides. In certain embodiments, it may be desirable to introduce the polynucleotide constructs into a host cell and assay an expression product for a structural and/or functional characteristic.

In yet another aspect, the invention provides methods for assembling, in a single pool, two or more polynucleotide constructs having at least one region of internal homology based on methods that permit distinction between correct assembly products as compared to incorrect cross-over products. For example, in one embodiment, the construction oligonucleotides may be designed to contain a distinguishable complement of sequence tags such that correctly assembled products may be distinguished from incorrect assembly products on the basis of size (e.g., using a column or a gel). Alternatively, the 5′ and 3′ most terminal construction oligonucleotides may be designed to contain complementary sequences which permit circularization of the correctly circularized products while the incorrect cross-over products remain linear. The circularized products may then be separated from the linear products on the basis of size or by using an exonuclease to destroy the linear product. In certain embodiments, a bridging oligonucleotide may be used to facilitate circularization of the correctly assembled products.

In yet another aspect, the invention provides a composition comprising a plurality of construction oligonucleotides wherein at least a portion of said construction oligonucleotides comprise a MutH cut site flanking the construction oligonucleotide at the 5′ end, 3′ end, or both ends. In certain embodiments, at least a portion of said construction oligonucleotides further comprise at least one or more of the following: (i) at least one pair of primer hybridization sites flanking the construction oligonucleotides and common to at least a subset of said construction oligonucleotides, (ii) at least one cleavage site between the construction oligonucleotide and any flanking sequence and common to at least a subset of said construction oligonucleotides, and/or (iii) a tag (such as, for example, biotin, fluorescein, or an aptamer) common to at least a subset of said construction oligonucleotides.

In yet another aspect, the invention provides a process for a manufacturer to obtain customer orders for custom designed polynucleotide constructs in an automated process. For example, the method may comprise: (i) obtaining a desired sequence from the customer; (ii) computationally designing a set of construction oligonucleotides that define the desired sequence; and (iii) synthesizing the set of construction oligonucleotides. In certain embodiments, the methods may further comprise designing and synthesizing a set of selection oligonucleotides. The construction and/or selection oligonucleotides may be shipped to a customer for assembly at the destination. Alternatively, the manufacturer may further conduct the assembly process before shipping the final product to the customer.

In an exemplary embodiment, the construction and/or selection oligonucleotides may be synthesized on a solid support. The oligonucleotides may be amplified while attached to the support (e.g., the support serves as a template for production of copies of construction and/or selection oligonucleotides). Alternatively, the oligonucleotides may be severed from the solid support and optionally subjected to amplification.

In various embodiments, the polynucleotide constructs that may be assembled using the methods described herein may be at least about 1 kilobase, 10 kilobases, 100 kilobases, 1 megabase, or 1 gigabase in length, or longer. In certain embodiments, it may be desirable to insert the polynucleotide construct into a vector and/or a host cell. Additionally, it may be desirable to express one or more polypeptides from the polynucleotide construct (e.g., in a host cell, lysate, in vitro transcription/translation system, etc.).

In certain embodiments, the polynucleotide constructs produced by the methods described herein may have a base error rate of less than about 1 error in 500 bases, 1 error in 1,000 bases, 1 error in 10,000 bases, or better.

### BRIEF DESCRIPTION OF THE FIGURES

The foregoing and other features and advantages of the present invention will be more fully understood from the following detailed description of illustrative embodiments taken in conjunction with the accompanying drawings in which:

FIG. 1 shows Table 1 which displays the effects of error rates on polynucleotide fidelity.

FIG. 2 shows a schematic overview of one embodiment of a method for multiplex assembly of multiple polynucleotide constructs, from design of oligonucleotides to the production of a plurality of polynucleotide constructs having a predetermined sequence.

FIG. 3 illustrates three exemplary methods for assembly of construction oligonucleotides into subassemblies and/or polynucleotide constructs, including (A) ligation, (B) chain extension, and (C) chain extension plus ligation. The dotted lines represent strands that have been extended by polymerase.

FIG. 4 shows a schematic overview of one embodiment of a method for polynucleotide assembly that involves multiple rounds of assembly.

FIG. 5 shows a schematic overview of one embodiment of a method for polynucleotide assembly that utilizes universal primers to amplify an oligonucleotide pool.

FIG. 6 is a schematic overview demonstrating one embodiment of a method for polynucleotide assembly that utilizes one set of universal primers to amplify a pool of construction oligonucleotides and one set of universal primers to amplify a subassembly (e.g., abc).

FIG. 7 is a schematic overview showing one embodiment of a method for polynucleotide assembly that involves iterative rounds of error reduction and/or amplification and assembly.

FIG. 8 is a schematic overview demonstrating one method for increasing the efficiency of error reduction processes by subjecting an oligonucleotide pool to a round of denaturation/renaturation prior to error reduction. In the figure, Xs represent sequence errors (e.g., deviations from a desired sequence in the form of an insertion, deletion, or incorrect base).

FIG. 9 shows an illustration of various locations on a solid support with attached oligonucleotides; the inset shows that the center of the location contains higher fidelity oligonucleotides.

FIG. 10 is a schematic overview demonstrating one method for removing temporary primers using uracil-DNA glycosylase.

FIG. 11 illustrates possible crossover products that may arise when conducting multiplex assembly of polynucleotide constructs with internal homologous regions.

FIG. 12 illustrates crossover polymerization that may occur when conducting multiplex assembly of polynucleotide constructs with internal homologous regions.

FIG. 13 illustrates one embodiment of the circle selection method for multiplex assembly of polynucleotide constructs containing regions of homology.

FIG. 14 illustrates another embodiment of the circle selection method for multiplex assembly of polynucleotide constructs containing regions of homology.

FIG. 15 illustrates one embodiment of the size selection method for multiplex assembly of polynucleotide constructs containing regions of homology.

FIG. 16 illustrates another embodiment of the size selection method for multiplex assembly of polynucleotide constructs containing regions of homology.

FIG. 17 shows a schematic overview of one embodiment of a method for error filtration that is referred to as hybridization selection. 90-mer oligonucleotides (upper strands black, lower strands grey) are cut with type IIS restriction enzymes to release hybrids of 50-mers and complementary 44-mers, some of which have incorrect sequences (indicated by a bulge in the upper strand of the second 90-mer oligonucleotide). Only the correct upper 50-mer strand hybridizes well with left (L) then right (R) selection oligonucleotides (immobilized on beads in gray).

FIG. 18 illustrates one method for removal of error sequences using mismatch binding proteins.

FIG. 19 illustrates another method for removal of error sequences using mismatch binding proteins and universal tags containing cut sites for mismatch repair enzymes.

FIG. 20 illustrates neutralization of error sequences with mismatch recognition proteins.

FIG. 21 illustrates one method for strand-specific error correction.

FIG. 22 illustrates one method for local removal of DNA on both strands at the site of a mismatch.

FIG. 23 illustrates another method for local removal of DNA on both strands at the site of a mismatch.

FIG. 24 illustrates an exemplary mismatch binding agent that may be used to cleave oligonucleotides having a base error (mismatch). (A) shows one type of MMBP-N (mismatch binding protein-nuclease fusion protein), e.g., a FokI-MutS fusion, that may be used in accordance with the error reduction methods disclosed herein. (B) shows an exemplary method for removal of the error sequences from a reaction mixture. The reaction is conducted in a chamber separated by a membrane having a size barrier such that only the small excised pieces of DNA may pass through the filter. The filter preferably has affinity for the DNA pieces that pass through the membrane thereby retaining the small pieces and removing them from the reaction mixture.

FIG. 25 summarizes the effects of the methods of FIG. 18 applied to two DNA duplexes, each containing a single base (mismatch) error.

FIG. 26 shows an example of semi-selective removal of mismatch-containing segments.

FIG. 27 shows a procedure for reducing correlated errors in synthesized DNA.

FIG. 28 depicts a schematic of software useful in designing a set of construction oligonucleotides, selection oligonucleotides, and/or an assembly strategy.

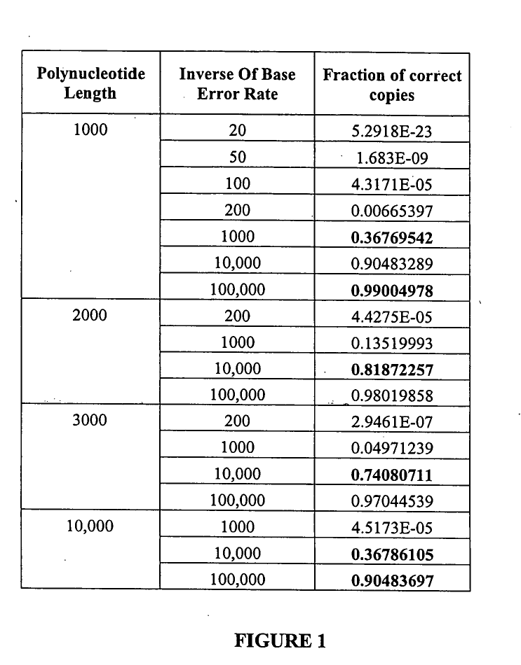

### DETAILED DESCRIPTION

1. Definitions As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “AlkA” refers to a 3-methyladenine DNA glycosylase II that corrects 5-formyluracil (fU)/G mispairs. Exemplary AlkA proteins include, for example, polypeptides encoded by nucleic acids having the following GenBank accession Nos.: D14465 (Bacillus subtilis) and K02498 (E. coli) as well as homologs, orthologs, paralogs, variants, or fragments thereof.

The term “amplification” means that the number of copies of a nucleic acid fragment is increased.

The term “AP endonuclease” refers to an endonuclease that recognizes an abasic (e.g., apurinic or apyrimidinic) site in a DNA duplex and removes the ribose-phosphate moiety from the backbone forming a single stranded break. Abasic sites may be formed by DNA glycosylases, such as, for example, Ura-DNA-glycosylase (recognizes uracil bases), thymine-DNA glycosylase (recognizes G/T mismatches), and Mut Y (recognizes G/A mismatches). Exemplary AP endonucleases include, for example, APE 1 (or HAP 1 or Ref-1), Endonuclease III, Endonuclease IV, Endonuclease VIII, Fpg, or Hogg1, all of which are commercially available, for example, from New England Biolabs (Beverly, Mass.).

The phrase “attenuated virus”, as used herein, means that the infection of a susceptible host by that virus will result in decreased probability of causing a disease in its host (loss of virulence) in accord with standard terminology in the art. See, e.g., B. Davis, R. Dulbecco, H. Eisen, and H. Ginsberg, Microbiology, 132 (3rd ed. 1980).

The term “base-pairing” refers to the specific hydrogen bonding between purines and pyrimidines in double-stranded nucleic acids including, for example, adenine (A) and thymine (T), guanine (G) and cytosine (C), (A) and uracil (U), and guanine (G) and cytosine (C), and the complements thereof. Base-pairing leads to the formation of a nucleic acid double helix from two complementary single strands.

The term “cleavage” as used herein refers to the breakage of a bond between two nucleotides, such as a phosphodiester bond.

The terms “comprise” and “comprising” are used in the inclusive, open sense, meaning that additional elements may be included.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

The term “construction oligonucleotide” refers to a single stranded oligonucleotide that may be used for assembling nucleic acid molecules that are longer than the construction oligonucleotide itself. In exemplary embodiments, a construction oligonucleotide may be used for assembling a nucleic acid molecule that is at least about 3-fold, 4-fold, 5-fold, 10-fold, 20-fold, 50-fold, 100-fold, or more, longer than the construction oligonucleotide. Typically a set of different construction oligonucleotides having predetermined sequences will be used for assembly into a larger nucleic acid molecule having a desired sequence. In exemplary embodiments, construction oligonucleotides may be from about 25 to about 200, about 50 to about 150, about 50 to about 100, or about 50 to about 75 nucleotides in length. Assembly of construction oligonucleotides may be carried out by a variety of methods including, for example, PAM, PCR assembly, ligation chain reaction, ligation/fusion PCR, dual asymmetrical PCR, overlap extension PCR, and combinations thereof. Construction oligonucleotides may be single stranded oligonucleotides or double stranded oligonucleotides. In an exemplary embodiment, construction oligonucleotides are synthetic oligonucleotides that have been synthesized in parallel on a substrate. Sequence design for construction oligonucleotides may be carried out with the aid of a computer program such as, for example, DNAWorks (Hoover and Lubkowski, Nucleic Acids Res. 30: e43 (2002), Gene2Oligo (Rouillard et al., Nucleic Acids Res. 32: W176-180 (2004) and world wide web at berry.engin.umich.edu/gene2oligo), or the implementation systems and methods discussed further below.

The term “dam” refers to an adenine methyltransferases that plays a role in coordinating DNA replication initiation, DNA mismatch repair and the regulation of expression of some genes. The term is meant to encompass prokaryotic dam proteins as well as homologs, orthologs, paralogs, variants, or fragments thereof. Exemplary dam proteins include, for example, polypeptides encoded by nucleic acids having the following GenBank accession Nos. AF091142 (Neisseria meningitidus strain BF13), AF006263 (Treponema pallidum), U76993 (Salmonella typhimurium) and M22342 (Bacteriphage T2).

The terms “denature” or “melt” refer to a process by which strands of a duplex nucleic acid molecule are separated into single stranded molecules. Methods of denaturation include, for example, thermal denaturation and alkaline denaturation.

The term “detectable marker” refers to a polynucleotide sequence that facilitates the identification of a cell harboring the polynucleotide sequence. In certain embodiments, the detectable marker encodes for a chemiluminescent or fluorescent protein, such as, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED). In other embodiments, the detectable marker may be an antigenic or affinity tag such as, for example, a polyHis tag, myc, HA, GST, protein A, protein G, calmodulin-binding peptide, thioredoxin, maltose-binding protein, poly arginine, poly His-Asp, FLAG, etc.

The term “DNA repair” refers to a process wherein sequence errors in a nucleic acid (DNA:DNA duplexes, DNA:RNA and, for purposes herein, also RNA:RNA duplexes) are recognized by a nuclease that excises the damaged or mutated region from the nucleic acid; and then further enzymes or enzymatic activities synthesize a replacement portion of a strand(s) to produce the correct sequence.

The term “DNA repair enzyme” refers to one or more enzymes that correct errors in nucleic acid structure and sequence, i.e., recognizes, binds and corrects abnormal base-pairing in a nucleic acid duplex. Examples of DNA repair enzymes include, for example, mutH, mutL, mutM, mutS, mutY, dam, thymidine DNA glycosylase (TDG), uracil DNA glycosylase, AlkA, MLH1, MSH2, MSH3, MSH6, Exonuclease I, T4 endonuclease V, Exonuclease V, RecJ exonuclease, FEN1 (RAD27), dnaQ (mutD), polC (dnaE), or combinations thereof, as well as homologs, orthologs, paralogs, variants, or fragments of the forgoing. Enzymatic systems capable of recognition and correction of base pairing errors within the DNA helix have been demonstrated in bacteria, fungi and mammalian cells.

The term “duplex” refers to a nucleic acid molecule that is at least partially double stranded. A “stable duplex” refers to a duplex that is relatively more likely to remain hybridized to a complementary sequence under a given set of hybridization conditions. In an exemplary embodiment, a stable duplex refers to a duplex that does not contain a basepair mismatch, insertion, or deletion. An “unstable duplex” refers to a duplex that is relatively less likely to remain hybridized to a complementary sequence under a given set of hybridization conditions. In an exemplary embodiment, an unstable duplex refers to a duplex that contains at least one basepair mismatch, insertion, or deletion.

The term “error reduction” refers to process that may be used to reduce the number of sequence errors in a nucleic acid molecule, or a pool of nucleic acid molecules, thereby increasing the number of error free copies in a composition of nucleic acid molecules. Error reduction includes error filtration, error neutralization, and error correction processes. “Error filtration” is a process by which nucleic acid molecules that contain a sequence error are removed from a pool of nucleic acid molecules. Methods for conducting error filtration include, for example, hybridization to a selection oligonucleotide, or binding to a mismatch binding agent, followed by separation. “Error neutralization” is a process by which a nucleic acid containing a sequence error is restricted from amplifying and/or assembling but is not removed from the pool of nucleic acids. Methods for error neutralization include, for example, binding to a mismatch binding agent and optionally covalent linkage of the mismatch binding agent to the DNA duplex. “Error correction” is a process by which a sequence error in a nucleic acid molecule is corrected (e.g., an incorrect nucleotide at a particular location is changed to the nucleic acid that should be present based on the predetermined sequence). Methods for error correction include, for example, homologous recombination or sequence correction using DNA repair proteins.

The term “gene” refers to a nucleic acid comprising an open reading frame encoding a polypeptide having exon sequences and optionally intron sequences. The term “intron” refers to a DNA sequence present in a given gene which is not translated into protein and is generally found between exons.

The term “hybridize” or “hybridization” refers to specific binding between two complementary nucleic acid strands. In various embodiments, hybridization refers to an association between two perfectly matched complementary regions of nucleic acid strands as well as binding between two nucleic acid strands that contain one or more mismatches (including mismatches, insertion, or deletions) in the complementary regions. Hybridization may occur, for example, between two complementary nucleic acid strands that contain 1, 2, 3, 4, 5, or more mismatches. In various embodiments, hybridization may occur, for example, between partially overlapping and complementary construction oligonucleotides, between partially overlapping and complementary construction and selection oligonucleotides, between a primer and a primer binding site, etc. The stability of hybridization between two nucleic acid strands may be controlled by varying the hybridization conditions and/or wash conditions, including for example, temperature and/or salt concentration. For example, the stringency of the hybridization conditions may be increased so as to achieve more selective hybridization, e.g., as the stringency of the hybridization conditions are increased the stability of binding between two nucleic acid strands, particularly strands containing mismatches, will be decreased.

The term “including” is used to mean “including but not limited to”. “Including” and “including but not limited to” are used interchangeably.

The term “ligase” refers to a class of enzymes and their functions in forming a phosphodiester bond in adjacent oligonucleotides which are annealed to the same oligonucleotide. Particularly efficient ligation takes place when the terminal phosphate of one oligonucleotide and the terminal hydroxyl group of an adjacent second oligonucleotide are annealed together across from their complementary sequences within a double helix, i.e. where the ligation process ligates a “nick” at a ligatable nick site and creates a complementary duplex (Blackburn, M. and Gait, M. (1996) in Nucleic Acids in Chemistry and Biology, Oxford University Press, Oxford, pp. 132-33, 481-2). The site between the adjacent oligonucleotides is referred to as the “ligatable nick site”, “nick site”, or “nick”, whereby the phosphodiester bond is non-existent, or cleaved.

The term “ligate” refers to the reaction of covalently joining adjacent oligonucleotides through formation of an internucleotide linkage.

The terms “mismatch binding agent” or “MMBA” refer to an agent that binds to a double stranded nucleic acid molecule that contains a mismatch. The agent may be chemical or proteinacious. In an exemplary embodiment, a MMBA is a mismatch binding protein (MMBP) such as, for example, Fok I, MutS, T7 endonuclease, a DNA repair enzyme as described herein, a mutant DNA repair enzyme as described in U.S. Patent Publication No. 2004/0014083, or fragments or fusions thereof Mismatches that may be recognized by an MMBA include, for example, one or more nucleotide insertions or deletions, or improper base pairing, such as A:A, A:C, A:G, C:C, C:T, G:G, G:T, T:T, C:U, G:U, T:U, U:U, 5-formyluracil (fU):G, 7,8-dihydro-8-oxo-guanine (8-oxoG):C, 8-oxoG:A or the complements thereof.

The term “MLH1” and “PMS1” (PMS2 in humans) refers to the components of the eukaryotic mutL-related protein complex, e.g., MLH1-PMS1, that interacts with MSH2-containing complexes bound to mispaired bases. Exemplary MLH1 proteins include, for example, polypeptides encoded by nucleic acids having the following GenBank accession Nos. AI389544 (Drosophila melanogaster), AI387992 (Drosophila melanogaster), AF068257 (Drosophila melanogaster), U80054 (Rattus norvegicus) and U07187 (Saccharomyces cerevisiae), as well as homologs, orthologs, paralogs, variants, or fragments thereof.

The term “MSH2” refers to a component of the eukaryotic DNA repair complex that recognizes base mismatches and insertion or deletion of up to 12 bases. MSH2 forms heterodimers with MSH3 or MSH6. Exemplary MSH2 proteins include, for example, polypeptides encoded by nucleic acids having the following GenBank accession Nos.: AF109243 (Arabidopsis thaliana), AF030634 (Neurospora crassa), AF002706 (Arabidopsis thaliana), AF026549 (Arabidopsis thaliana), L47582 (Homo sapiens), L47583 (Homo sapiens), L47581 (Homo sapiens) and M84170 (S. cerevisiae) and homologs, orthologs, paralogs, variants, or fragments thereof. Exemplary MSH3 proteins include, for example, polypeptides encoded by the nucleic acids having GenBank accession Nos.: J04810 (Human) and M96250 (Saccharomyces cerevisiae) and homologs, orthologs, paralogs, variants, or fragments thereof. Exemplary MSH6 proteins include, for example, polypeptide encoded by nucleic acids having the following GenBank accession Nos.: U54777 (Homo sapiens) and AF031087 (Mus musculus) and homologs, orthologs, paralogs, variants, or fragments thereof.

The term “mutH” refers to a latent endonuclease that incises the unmethylated strand of a hemimethylated DNA, or makes a double strand cleavage on unmethylated DNA, 5′ to the G of d(GATC) sequences or. The term is meant to include prokaryotic mutH (e.g., Welsh et al., 262 J. Biol. Chem. 15624 (1987)) as well as homologs, orthologs, paralogs, variants, or fragments thereof.

The term “mutHLS” refers to a complex between mutH, mutL, and mutS proteins (or homologs, orthologs, paralogs, variants, or fragments thereof).

The term “mutL” refers to a protein that couples abnormal base-pairing recognition by mutS to mutH incision at the 5′-GATC-3′ sequences in an ATP-dependent manner. The term is meant to encompass prokaryotic mutL proteins as well as homologs, orthologs, paralogs, variants, or fragments thereof. Exemplary mutL proteins include, for example, polypeptides encoded by nucleic acids having the following GenBank accession Nos. AF170912 (Caulobacter crescentus), AI518690 (Drosophila melanogaster), AI456947 (Drosophila melanogaster), AI389544 (Drosophila melanogaster), AI387992 (Drosophila melanogaster), AI292490 (Drosophila melanogaster), AF068271 (Drosophila melanogaster), AF068257 (Drosophila melanogaster), U50453 (Thermus aquaticus), U27343 (Bacillus subtilis), U71053 (U71053 (Thermotoga maritima), U71052 (Aquifex pyrophilus), U13696 (Human), U13695 (Human), M29687 (S.typhimurium), M63655 (E. coli) and L19346 (Escherichia coli). Exemplary mutL homologs include, for example, eukaryotic MLH1, MLH2, PMS1, and PMS2 proteins (see e.g., U.S. Pat. Nos. 5,858,754 and 6,333,153).

The term “mutM” refers to an 8-oxoguanine DNA glycosylase that removes 7,8-dihydro-8-oxoguanine (8-oxoG) and formamido pyrimidine (Fapy) lesions from DNA. Exemplary mutM proteins include, for example, polypeptides encoded by nucleic acids having the following GenBank accession Nos. AF148219 (Nostoc PCC8009), AF026468 (Streptococcus mutans), AF093820 (Mastigocladus laminosus), AB010690 (Arabidopsis thaliana), U40620 (Streptococcus mutans), AB008520 (Thermus thermophilus) and AF026691 (Homo sapiens), as well as homologs, orthologs, paralogs, variants, or fragments thereof.

The term “mutS” refers to a DNA-mismatch binding protein that recognizes and binds to a variety of mispaired bases and small (1-5 bases) single-stranded loops. The term is meant to encompass prokaryotic mutS proteins as well as homologs, orthologs, paralogs, variants, or fragments thereof. The term also encompasses homo- and hetero-dimmers and multimers of various mutS proteins. Exemplary mutS proteins include, for example, polypeptides encoded by nucleic acids having the following GenBank accession Nos. AF146227 (Mus musculus), AF193018 (Arabidopsis thaliana), AF144608 (Vibrio parahaemolyticus), AF034759 (Homo sapiens), AF104243 (Homo sapiens), AF007553 (Thermus aquaticus caldophilus), AF109905 (Mus musculus), AF070079 (Homo sapiens), AF070071 (Homo sapiens), AH006902 (Homo sapiens), AF048991 (Homo sapiens), AF048986 (Homo sapiens), U33117 (Thermus aquaticus), U16152 (Yersinia enterocolitica), AF000945 (Vibrio cholarae), U698873 (Escherichia coli), AF003252 (Haemophilus influenzae strain b (Eagan), AF003005 (Arabidopsis thaliana), AF002706 (Arabidopsis thaliana), L10319 (Mouse), D63810 (Thermus thermophilus), U27343 (Bacillus subtilis), U71155 (Thermotoga maritima), U71154 (Aquifex pyrophilus), U16303 (Salmonella typhimurium), U21011 (Mus musculus), M84170 (S. cerevisiae), M84169 (S. cerevisiae), M18965 (S. typhimurium) and M63007 (Azotobacter vinelandii). Exemplary mutS homologs include, for example, eukaryotic MSH2, MSH3, MSH4, MSH5, and MSH6 proteins (see e.g., U.S. Pat. Nos. 5,858,754 and 6,333,153).

The term “mutY” refers to an adenine glycosylase that is involved in the repair of 7,8-dihydro-8-oxo-2′-deoxyguanosine (OG):A and G:A mispairs in DNA. Exemplary mutY proteins include, for example, polypeptides encoded by nucleic acids having the following GenBank accession Nos. AF121797 (Streptomyces), U63329 (Human), AA409965 (Mus musculus) and AF056199 (Streptomyces), as well as homologs, orthologs, paralogs, variants, or fragments thereof.

The terms “nucleic acid” or “polynucleotide” refer to a polymeric form of nucleotides, either ribonucleotides and/or deoxyribonucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “oligonucleotide” refers to a short nucleic acid molecule, e.g., a nucleic acid molecule having from about 10 to about 200 nucleotides. Oligonucleotides may be single stranded or double stranded.

The term “operably linked”, when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (e.g., inducers and polymerases) are bound to the control or regulatory sequence(s).

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “polynucleotide construct” refers to a long nucleic acid molecule having a predetermined sequence. Polynucleotide constructs may be assembled from a set of construction oligonucleotides and/or a set of subassemblies.

A “region of internal homology” refers to an internal portion of a sequence that has substantial identity with an internal portion of another sequence, e.g., portions of the sequences of two subassemblies, portions of the sequences of two polynucleotide constructs, etc. An internal portion means that the homologous sequence portion does not encompass either the 5′ or the 3′ terminal most sequences of the polynucleotide. The degree of homology between the internal sequence portions is sufficiently high to permit hybridization between complementary strands of the sequence portions under conditions suitable for polynucleotide assembly as described herein. For example, the regions of internal homology may comprise at least about 70%, 75%, 80%, 85%, 90%, 95%, 96%, 96%, 98%, 99% or 100% sequence identity. The region of internal homology may span at least about 10, 15, 20, 25, 30, 40, 50, 60, 70, 80, 90, 100, or more, consecutive nucleic acid residues. In an exemplary embodiment, the region of internal homology spans at least the length of a construction oligonucleotide.

The term “restriction endonuclease recognition site” refers to a nucleic acid sequence capable of binding one ore more restriction endonucleases. The term “restriction endonuclease cleavage site” refers to a nucleic acid sequence that is cleaved by one or more restriction endonucleases. For a given enzyme, the restriction endonuclease recognition and cleavage sites may the same or different. Restriction enzymes include, but are not limited to, type I enzymes, type II enzymes, type IIS enzymes, type III enzymes and type IV enzymes. The REBASE database provides a comprehensive database of information about restriction enzymes, DNA methyltransferases and related proteins involved in restriction-modification. It contains both published and unpublished work with information about restriction endonuclease recognition sites and restriction endonuclease cleavage sites, isoschizomers, commercial availability, crystal and sequence data (see Roberts RJ et al. (2005) REBASE—restriction enzymes and DNA methyltransferases. Nucleic Acids Res.;33 Database Issue:D230-2).

The term “selectable marker” refers to a polynucleotide sequence encoding a gene product that alters the ability of a cell harboring the polynucleotide sequence to grow or survive in a given growth environment relative to a similar cell lacking the selectable marker. Such a marker may be a positive or negative selectable marker. For example, a positive selectable marker (e.g., an antibiotic resistance or auxotrophic growth gene) encodes a product that confers growth or survival abilities in selective medium (e.g., containing an antibiotic or lacking an essential nutrient). A negative selectable marker, in contrast, prevents polynucleotide-harboring cells from growing in negative selection medium, when compared to cells not harboring the polynucleotide. A selectable marker may confer both positive and negative selectability, depending upon the medium used to grow the cell. The use of selectable markers in prokaryotic and eukaryotic cells is well known by those of skill in the art. Suitable positive selection markers include, e.g., neomycin, kanamycin, hyg, hisD, gpt, bleomycin, tetracycline, hprt SacB, beta-lactamase, ura3, ampicillin, carbenicillin, chloramphenicol, streptamycin, gentamycin, phleomycin, and nalidixic acid. Suitable negative selection markers include, e.g., hsv-tk, hprt, gpt, and cytosine deaminase.

The term “selection oligonucleotide” refers to a single stranded oligonucleotide that is complementary to at least a portion of a construction oligonucleotide (or the complement of the construction oligonucleotide). Selection oligonucleotides may be used for removing copies of a construction oligonucleotide that contain sequencing errors (e.g., a deviation from the desired sequence) from a pool of construction oligonucleotides. In an exemplary embodiment, a selection oligonucleotide may be end immobilized on a substrate. In one embodiment, selection oligonucleotides are synthetic oligonucleotides that have been synthesized in parallel on a substrate. Preferably, selection oligonucleotides are complementary to at least about 20%, 25%, 30%, 50%, 60%, 70%, 80%, 90%, or 100% of the length of the construction oligonucleotide (or the complement of the construction oligonucleotide). In an exemplary embodiment, a pool of selection oligonucleotides is designed such that the melting temperature (Tm) of a plurality of construction/selection oligonucleotide pairs is substantially similar. In one embodiment, a pool of selection oligonucleotides is designed such that the melting temperature of substantially all of the construction/selection oligonucleotides pairs is substantially similar. For example, the melting temperature of at least about 50%, 60%, 70%, 75%, 80%, 90%, 95%, 97%, 98%, 99%, or greater, of the construction/selection oligonucleotide pairs is within about 10° C., 7° C., 5° C., 4° C., 3° C., 2° C., 1° C., or less, of each other. Sequence design for selection oligonucleotides may be carried out with the aid of a computer program such as, for example, DNAWorks (Hoover and Lubkowski, Nucleic Acids Res. 30: e43 (2002), Gene2oligo (Rouillard et al., Nucleic Acids Res. 32: W176-180 (2004) and world wide web at berry.engin.umich.edu/gene2oligo), or the implementation systems and methods discussed further below.

The term “sequence homology” refers to the proportion of base matches between two nucleic acid sequences or the proportion of amino acid matches between two amino acid sequences. When sequence homology is expressed as a percentage, e.g., 50%, the percentage denotes the proportion of matches over the length of a desired sequence as compared to another sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids occurs in both sequences to yield the number of matched positions, dividing the number of matched positions by the total number of positions in the comparison window, and multiplying the result by 100 to yield the percentage of sequence identity. Methods to calculate sequence identity are known to those of skill in the art and described in further detail below.

The terms “stringent conditions” or “stringent hybridization conditions” refer to conditions which promote specific hybridization between two complementary polynucleotide strands so as to form a duplex. Stringent conditions may be selected to be about 5° C. lower than the thermal melting point (Tm) for a given polynucleotide duplex at a defined ionic strength and pH. The length of the complementary polynucleotide strands and their GC content will determine the Tm of the duplex, and thus the hybridization conditions necessary for obtaining a desired specificity of hybridization. The Tm is the temperature (under defined ionic strength and pH) at which 50% of a polynucleotide sequence hybridizes to a perfectly matched complementary strand. In certain cases it may be desirable to increase the stringency of the hybridization conditions to be about equal to the Tm for a particular duplex.

A variety of techniques for estimating the Tm are available. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex. Other methods for calculating Tm are described in SantaLucia and Hicks, Annu. Rev. Biomol. Struct. 33: 415-40 (2004) using the formula Tm=ΔH°×1000/(ΔS°+R×ln(C_{T}/x))−273.15, where C_{T} is the total molar strand concentration, R is the gas constant 1.9872 cal/K-mol, and x equals 4 for nonself-complementary duplexes and equals 1 for self-complementary duplexes.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may also include another agent affecting the stringency, for example, hybridization conducted in the presence of 50% formamide increases the stringency of hybridization at a defined temperature. In an exemplary embodiment, Betaine, e.g., about 5M Betaine, may be added to the hybridization reaction to minimize or eliminate the base pair composition dependence of DNA thermal melting transitions (see e.g., Rees et al., Biochemistry 32: 137-144 (1993)). In another embodiment, low molecular weight amides or low molecule weight sulfones (such as, for example, DMSO, tetramethylene sulfoxide, methyl sec-butyl sulfoxide, etc.) may be added to a hybridization reaction to reduce the melting temperature of sequences rich in GC content (see e.g., Chakarbarti and Schutt, BioTechniques 32: 866-874 (2002)).

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, e.g., 0.1 or 0.2% SDS. For example, hybridization may be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10×Denhardt (0.2% Ficoll, 0.2% Polyvinylpyrrolidone, 0.2% bovine serum albumin) and 200 μg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization may consist of hybridizing two nucleic acids in solution, or a nucleic acid in solution to a nucleic acid attached to a solid support, e.g., a filter. When one nucleic acid is on a solid support, a prehybridization step may be conducted prior to hybridization. Prehybridization may be carried out for at least about 1 hour, 3 hours or 10 hours in the same solution and at the same temperature as the hybridization solution (without the complementary polynucleotide strand).

Appropriate stringency conditions are known to those skilled in the art or may be determined experimentally by the skilled artisan. See, for example, Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press, N.Y; S. Agrawal (ed.) Methods in Molecular Biology, volume 20; Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York; Tibanyenda, N. et al., Eur. J. Biochem. 139:19 (1984) and Ebel, S. et al., Biochem. 31:12083 (1992); Rees et al., Biochemistry 32: 137-144 (1993); Chakarbarti and Schutt, BioTechniques 32: 866-874 (2002); and SantaLucia and Hicks, Annu. Rev. Biomol. Struct. 33: 415-40 (2004).

As applied to proteins, the term “substantial identity” means that two sequences, when optimally aligned, such as by the programs GAP or BESTFIT using default gap weights, typically share at least about 70 percent sequence identity, alternatively at least about 80, 85, 90, 95 percent sequence identity or more. For amino acid sequences, amino acid residues that are not identical may differ by conservative amino acid substitutions, which are described above.

The term “subassembly” refers to a nucleic acid molecule that has been assembled from a set of construction oligonucleotides. Preferably, a subassembly is at least about 3-fold, 4-fold, 5-fold, 10-fold, 20-fold, 50-fold, 100-fold, or more, longer than the construction oligonucleotide, e.g., about 300-600 bases long.

The term “synthetic,” as used herein with reference to a nucleic acid molecule, refers to production by in vitro chemical and/or enzymatic synthesis.

The term “TDG” refers to a thymine-DNA glycosylase that recognizes G/T mismatches. An exemplary TDG protein includes, for example, a polypeptide encoded by a nucleic acid having GenBank accession No. AF117602 (Ateles paniscus chamek), as well as homologs, orthologs, paralogs, variants, or fragments thereof. “Transcriptional regulatory sequence” is a generic term used herein to refer to DNA sequences, such as initiation signals, enhancers, and promoters, which induce or control transcription of protein coding sequences with which they are operable linked. In preferred embodiments, transcription of one of the recombinant genes is under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

As used herein, the term “transfection” means the introduction of a nucleic acid, e.g., an expression vector, into a recipient cell, and is intended to include commonly used terms such as “infect” with respect to a virus or viral vector. The term “transduction” is generally used herein when the transfection with a nucleic acid is by viral delivery of the nucleic acid. The term “transformation” refers to any method for introducing foreign molecules, such as DNA, into a cell. Lipofection, DEAE-dextran-mediated transfection, microinjection, protoplast fusion, calcium phosphate precipitation, retroviral delivery, electroporation, natural transformation, and biolistic transformation are just a few of the methods known to those skilled in the art which may be used.

The term “type-IIs restriction endonuclease” refers to a restriction endonuclease having a non-palindromic recognition sequence and a cleavage site that occurs outside of the recognition site (e.g., from 0 to about 20 nucleotides distal to the recognition site). Type IIs restriction endonucleases may create a nick in a double stranded nucleic acid molecule or may create a double stranded break that produces either blunt or sticky ends (e.g., either 5′ or 3′ overhangs). Examples of Type IIs endonucleases include, for example, enzymes that produce a 3′ overhang, such as, for example, Bsr I, Bsm I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57I, Eco57M I, Gsu I, Ppi I, and Psr I; enzymes that produce a 5′ overhang such as, for example, BsmA I, Ple I, Fau I, Sap I, BspM I, SfaN I, Hga I, Bvb I, Fok I, BceA I, BsmF I, Ksp632I, Eco31I, Esp3I, Aar I; and enzymes that produce a blunt end, such as, for example, Mly I and Btr I. Type-IIs endonucleases are commercially available and are well known in the art (New England Biolabs, Beverly, Mass.). Information about the recognition sites, cut sites and conditions for digestion using type IIs endonucleases may be found, for example, on the world wide web at neb.com/nebecomm/enzymefindersearchbytypeIIs.asp).

The term “universal tag” refers to a nucleotide sequence that flanks a plurality of polynucleotide sequences on the 5′ and/or 3′ termini, e.g., the tag is common to a plurality of polynucleotides. Universal tags may comprise one or more of the following: a primer hybridization sequence, a mismatch repair enzyme cut site, a restriction enzyme recognition site, a restriction enzyme cut site (or half site, e.g., half of the site is contained in the universal tag and half of the site is contained in the polynucleotide sequence), an aptamer, one or more uracil residues, one or more modified nucleic acid residues, or a label for detection and/or immobilization (e.g., biotin, fluorescein, etc.). In an exemplary embodiment, the universal tag comprises a mismatch repair enzyme cut site, such as, for example, the sequence GATC which is cut by the mutH endonuclease or the mutHLS complex. In certain embodiments, the universal tags may comprise binding sites for universal primers.

The term “universal primers” refers to a set of primers (e.g., a forward and reverse primer) that may be used for chain extension/amplification of a plurality of polynucleotides, e.g., the primers hybridize to sites that are common to a plurality of polynucleotides. For example, universal primers may be used for amplification of all, or essentially all, polynucleotides in a single pool, such as, for example, a pool of construction oligonucleotides, a pool of selection oligonucleotides, a pool of subassemblies, and/or a pool of polynucleotide constructs, etc. In one embodiment, a single primer may be used to amplify both the forward and reverse strands of a plurality of polynucleotides in a single pool. In certain embodiments, the universal primers may be temporary primers that may be removed after amplification via enzymatic or chemical cleavage. In other embodiments, the universal primers may comprise a modification that becomes incorporated into the polynucleotide molecules upon chain extension. Exemplary modifications include, for example, a 3′ or 5′ end cap, a label (e.g., fluorescein), or a tag (e.g., a tag that facilitates immobilization or isolation of the polynucleotide, such as, biotin, etc.).

The term “UDG” refers to a uracil-DNA glycosylase that removes free uracil from single stranded or double stranded DNA containing a uracil. Exemplary UDG proteins include, for example, polypeptides encoded by nucleic acids having the following GenBank accession Nos.: AF174292 (Schizosaccharomyces pombe), AF108378 (Cercopithecine herpesvirus), AF125182 (Homo sapiens), AF125181 (Xenopus laevis), U55041 (Homo sapiens), U55041 (Mus musculus), AF084182 (Guinea pig cytomegalovirus), U31857 (Bovine herpesvirus), AF022391 (Feline herpesvirus), M87499 (Human), J04434 (Bacteriophage PBS2), U13194 (Human herpesvirus 6), L34064 (Gallid herpesvirus 1), U04994 (Gallid herpesvirus 2), L01417 (Rabbit fibroma virus), M25410 (Herpes simplex virus type 2), J04470 (S.cerevisiae), J03725 (E.coli), U02513 (Suid herpesvirus), U02512 (Suid herpesvirus) and L13855 (Pseudorabies virus) as well as homologs, orthologs, paralogs, variants, or fragments thereof.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

2. Assembly of High Fidelity Long Nucleic Acid Molecules

In one aspect, the invention provides synthetic polynucleotides having high fidelity. The synthetic polynucleotides are at least about 1, 2, 3, 4, 5, 8, 10, 15, 20, 25, 30, 40, 50, 75, or 100 kilobases (kb), or 1 megabase (mb), or longer. In exemplary embodiments, a compositions of synthetic polynucleotides contains at least about 1%, 2%, 3%, 4%, 5%, 6%, 7%, 8%, 9%, 10%, 15%, 20%, 25%, 50%, 60%, 70%, 80%, 90%, or more, copies that are error free (e.g., having a sequence that does not deviate from a predetermined sequence). The percent of error free copies is based on the number of error free copies in the compositions as compared to the total number of copies of the polynucleotide in the composition that were intended to have the correct, e.g., predefined, sequence. In certain embodiments, a composition of synthetic polynucleotides contain at least about 10 femtomoles, 100 femtomoles, 1 nanomoles, 10 nanomoles, 100 nanomoles, 1 micromole, 10 micromoles, or more of polynucleotide constructs in the composition. In other embodiments, the composition may comprise large amounts of one or more nucleic acid products, e.g., at least about 1 milligram, 1 gram, 10 grams, 100 grams, 1 kilogram, or more. Such large scale preparations will be useful, for example, for the preparation of vaccines, gene therapy constructs, or other commercial applications. The synthetic polynucleotides are constructed in a cell free environment and therefore do not contain one or more cellular contaminants and/or modifications that may be associated with nucleic acids produced in vivo. For example, synthetic polynucleotides may be free, or essentially free, from one or more of the following: membrane components (e.g., lipids), lipopolysaccharides (LPS), carbohydrates, pyrogens, proteins (including, for example, DNA binding proteins, DNase, RNase, etc.), or DNA binding molecules, and/or modifications such as methylation. In an exemplary embodiment, a composition of synthetic polynucleotides is free of any protein other than a protein purposefully added to the composition during the process of preparing the polynucleotides, e.g., polymerase, a mismatch binding protein, a restriction endonuclease, ligase, an exonuclease, and/or an antibody, etc. In another embodiment, a composition of synthetic polynucleotides is free of any small molecule other than a small molecule purposefully added to the composition during the process of preparing the polynucleotides, e.g., dNTPs, biotin, and/or a chemical cross-linking agent, etc.

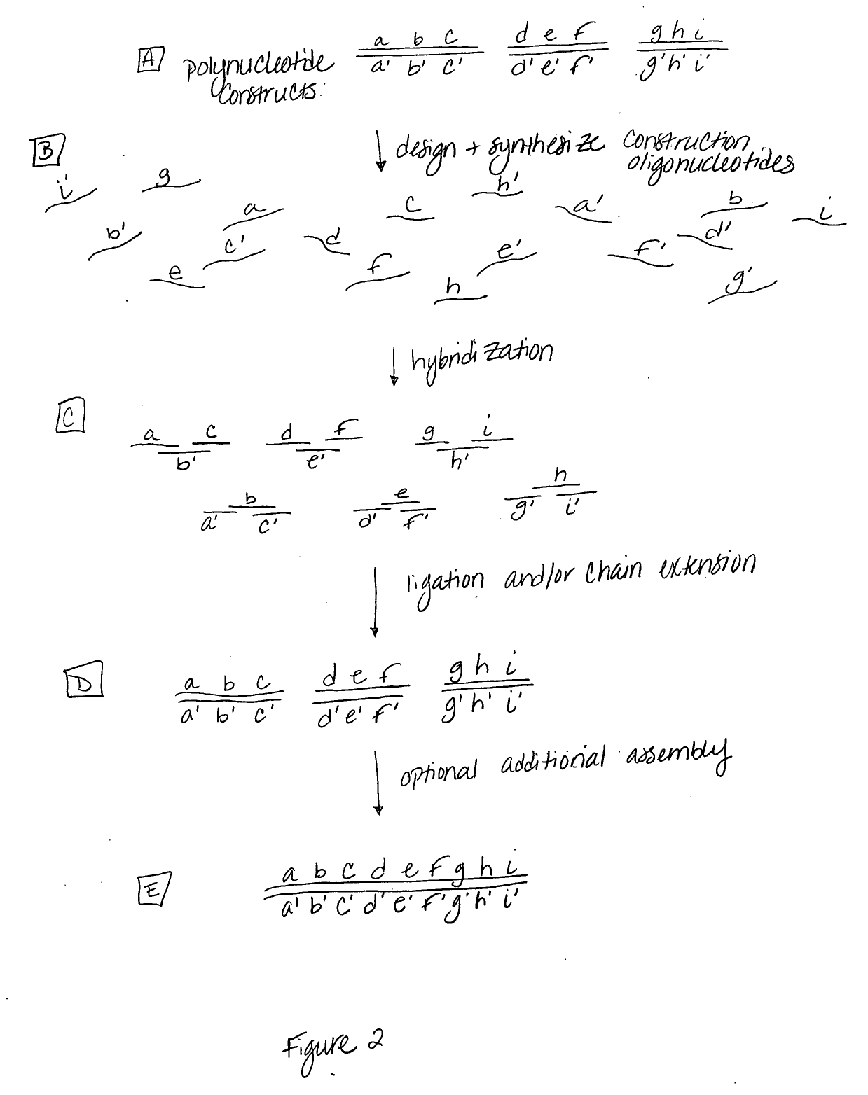

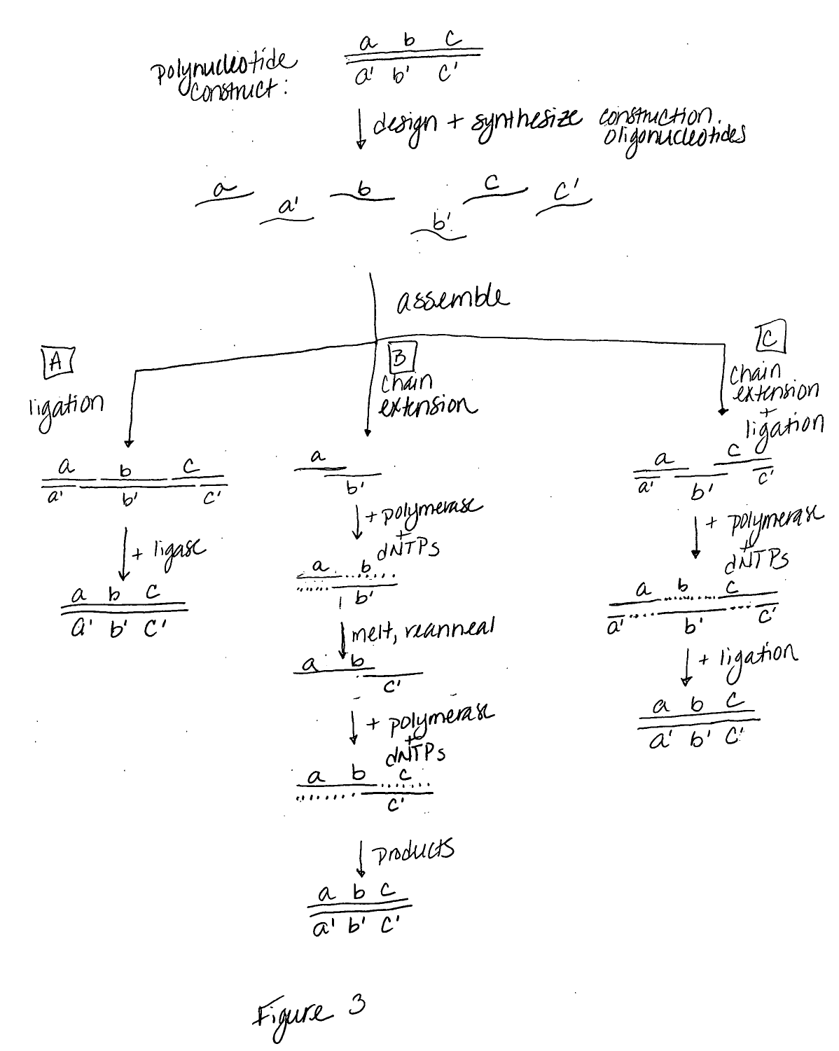

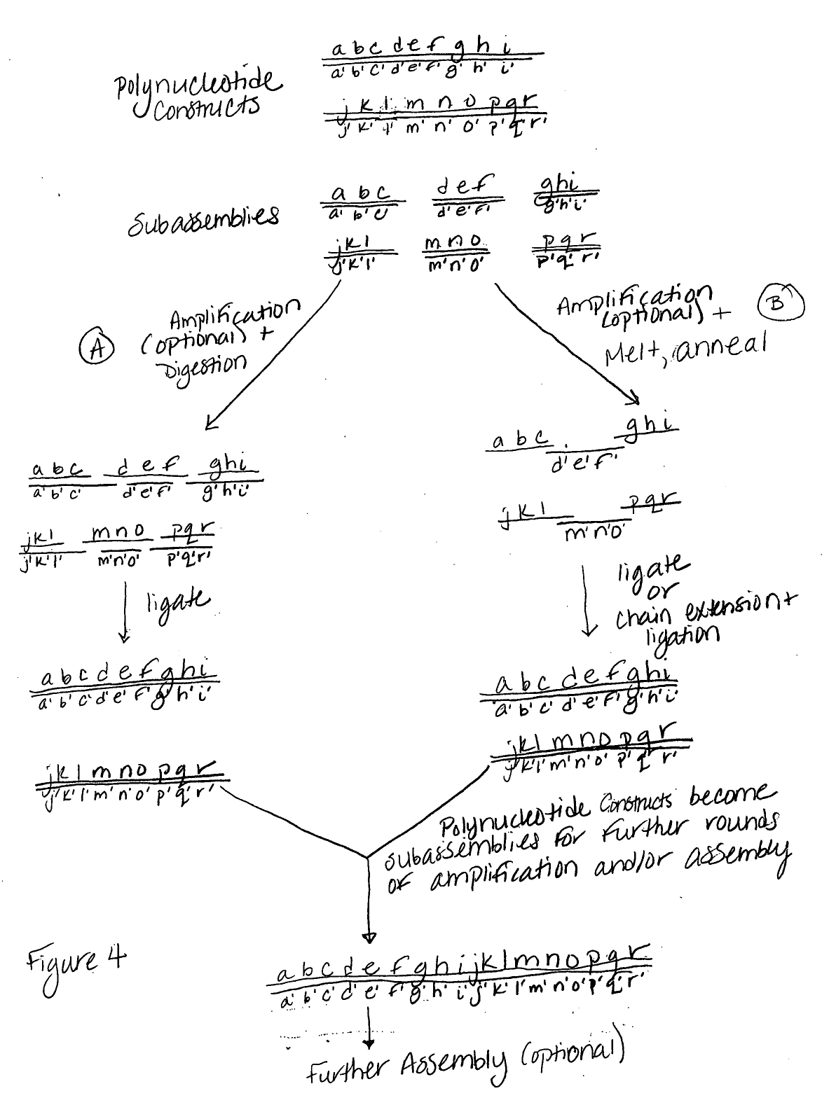

In another aspect, the invention provides methods for multiplex assembly of polynucleotide constructs, e.g., the assembly of two or more polynucleotide constructs having different predetermined sequences in a single reaction mixture. FIG. 2 provides one example of a multiplex assembly method provided herein. To produce two or more polynucleotide constructs having predetermined sequences, a set of construction oligonucleotides is designed that together cover the complete sequence of each of the polynucleotide constructs. The construction oligonucleotides are designed to have overlapping complementary regions that permit hybridization between complementary regions resulting in a properly ordered chain of construction oligonucleotides when mixed together under hybridization conditions (e.g., abc, def, and ghi in FIG. 2C). The assembly mixture is then subjected to ligation or polymerization and ligation to form a subassembly or polynucleotide construct (FIG. 2D). In certain embodiments, the construction oligonucleotides may cover the entire length of the polynucleotide construct so that when mixed together the oligos may simply be ligated together to form the subassembly/polynucleotide construct (e.g., FIG. 3A). Alternatively, the construction oligonucleotides may not be completely overlapping, but instead may leave gaps of single stranded regions that may be filled in with polymerase before ligation of the oligonucleotide segments into the subassembly/polynucleotide construct (FIG. 3C). Alternatively, the overlapping fragments may be sequentially extended through multiple rounds of denaturation/hybridization and chain extension until a full length product has been formed (see e.g., FIG. 3B). In certain embodiments, subassemblies of a variety of construction oligonucleotides may be further assembled into even longer polynucleotide constructs. For example, in one embodiment, the double stranded subassemblies may be melted and reannealed thus permitting hybridization between complementary regions of two or more subassemblies. The subassemblies can then be subjected to ligation or chain extension followed by ligation to form a polynucleotide construct formed of a set of subassemblies. Alternatively, the subassemblies may contain sequence specific sticky ends (e.g., 3′ or 5′ overhangs) that will permit joining of a variety of subassemblies in a desired order. The sticky ends may be formed through design of the construction oligonucleotides (e.g., the 5′ and/or 3′ most terminal construction oligonucleotides can be designed to have a single stranded overhang) or the subassemblies may be subjected to digestion with one or more restriction endonucleases to produce the sticky ends. After joining of the subassemblies via the sticky ends, the polynucleotide constructs may be formed by ligation and/or chain extension. The polynucleotide constructs formed from a set of subassemblies may optionally be subjected to further rounds of assembly to produce even longer polynucleotide constructs (see e.g., FIG. 4).

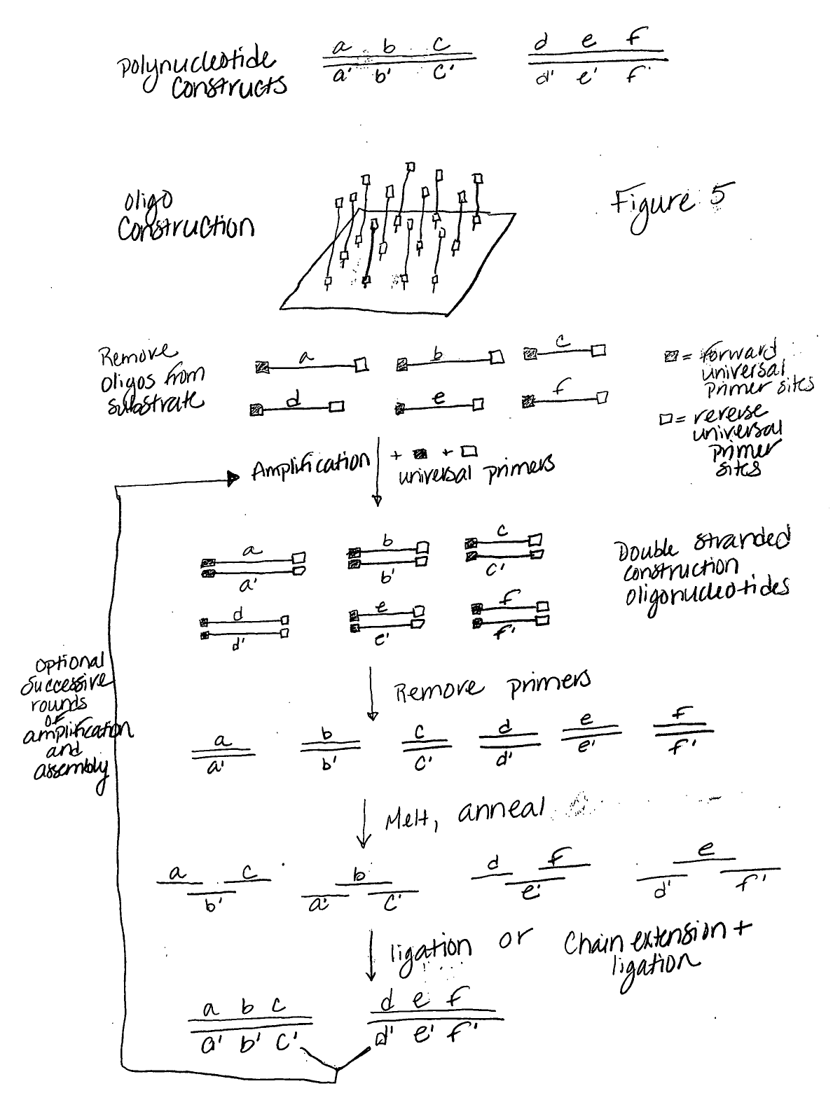

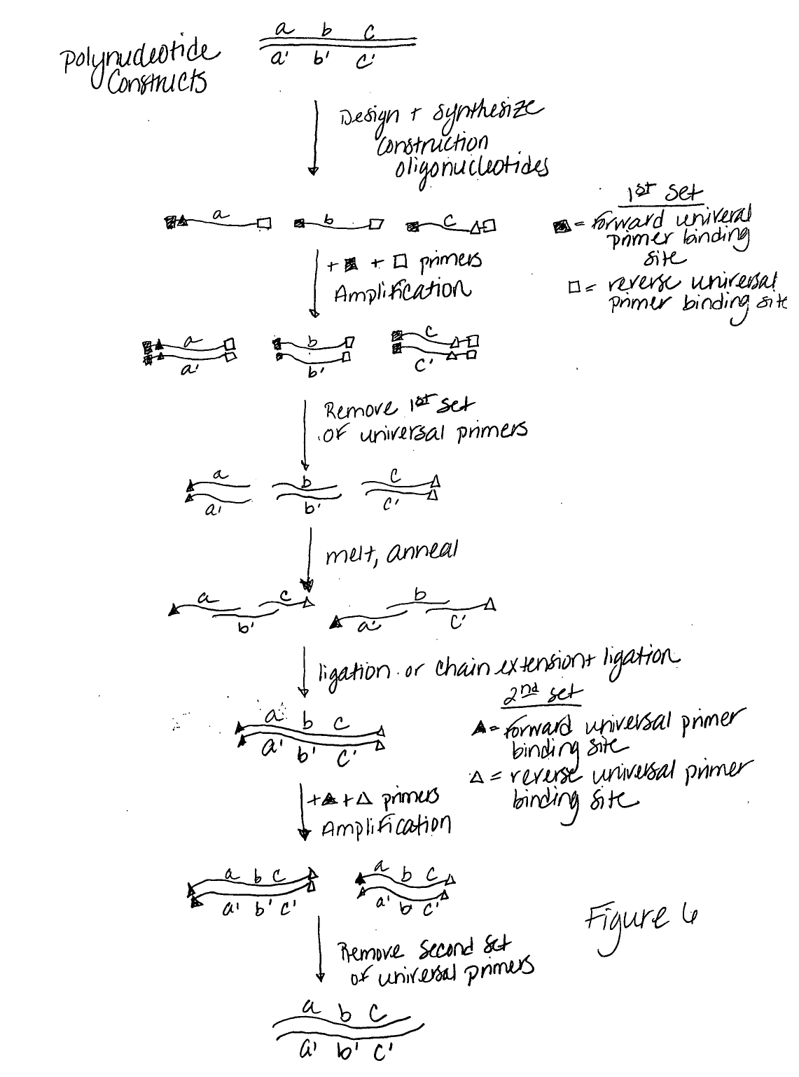

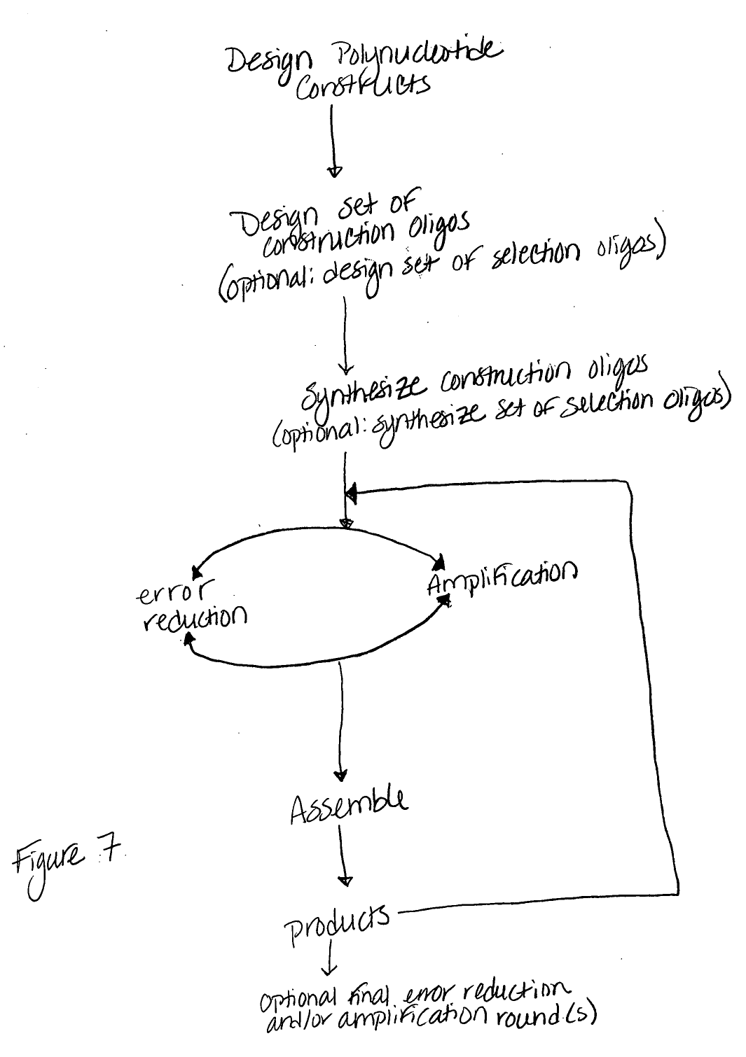

Also provided are methods for assembling polynucleotide constructs that involve amplification of polynucleotides at one or more steps using universal primers. For example, as shown in FIG. 5, construction oligonucleotides (e.g., a, b, c, d, e, and f) may be designed that comprise binding sites for universal primers (e.g., depicted as open and shaded squares). Before or after removal of construction oligonucleotides from the substrate, the entire pool may be amplified using a single set of universal primers. In an exemplary embodiment, the universal primers may be removed via enzymatic or chemical cleavage after amplification. The pool of amplified, construction oligonucleotides may then be melted, annealed and subjected to ligation and/or chain extension to form subassemblies (e.g., abc or def in FIG. 5). In certain embodiments, the subassemblies themselves may be amplified using a second set of universal primers (not shown). For example, the 5′ and 3′ most terminal construction oligonucleotides (e.g., a and d and c and f, respectively, in FIG. 5) may be designed to contain a second set of universal primer binding sites (see FIG. 6). Upon addition of the second set of universal primers to the subassembly pool, the plurality of subassemblies may be amplified. In an exemplary embodiment, the second set of universal primers may then be removed by chemical or enzymatic cleavage. The subassemblies may then be assembled into still longer polynucleotide constructs via hybridization of complementary strands or joining via sticky ends (as described above) followed by ligation and/or chain extension. This process may be repeated multiple times, e.g., successive rounds of amplification using universal primers (e.g., using a third set, fourth set, fifth set, etc.), cleavage of the primers, and assembly, until the desired polynucleotide construct has been formed. In exemplary embodiments, a plurality of assemblies may be carried out in a single reaction mixture. However, in certain embodiments, for example, when assembling a very large number of polynucleotide constructs, when assembling a set of highly homologous polynucleotide constructs, or when assembling a polynucleotide construct that contains one or more regions of internal homologies, it may be desirable to use a hierarchical assembly method. Various methods for hierarchical assembly are described below.

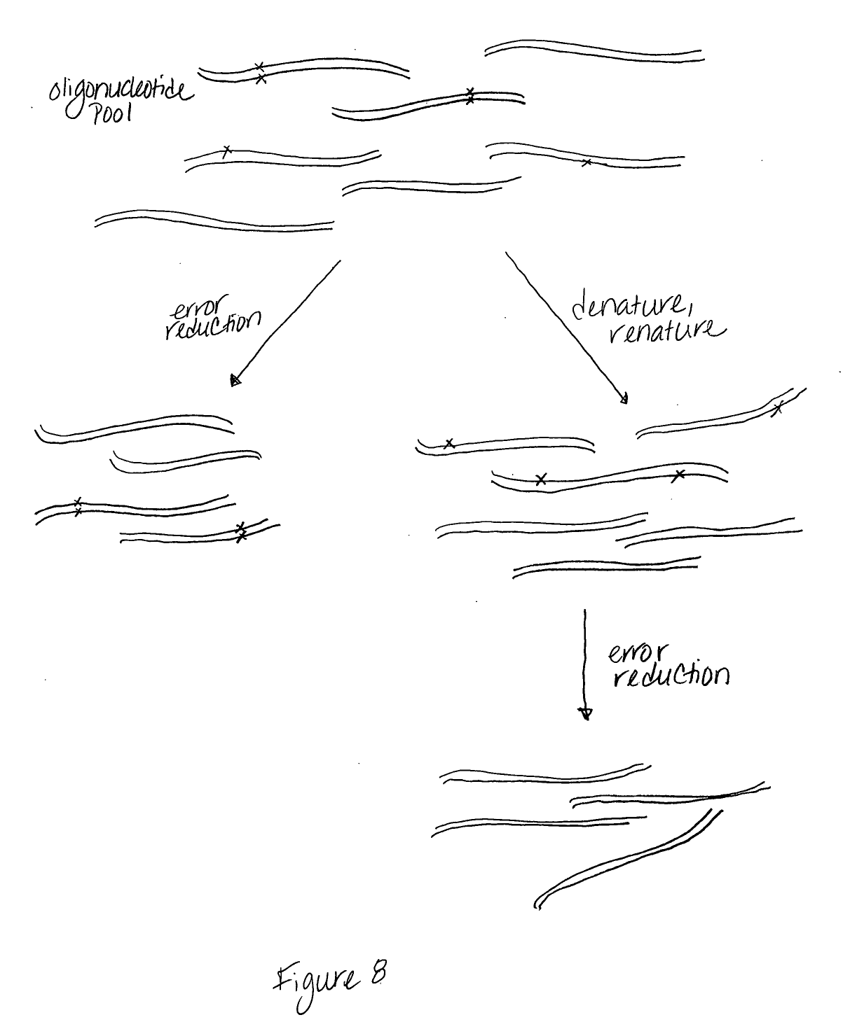

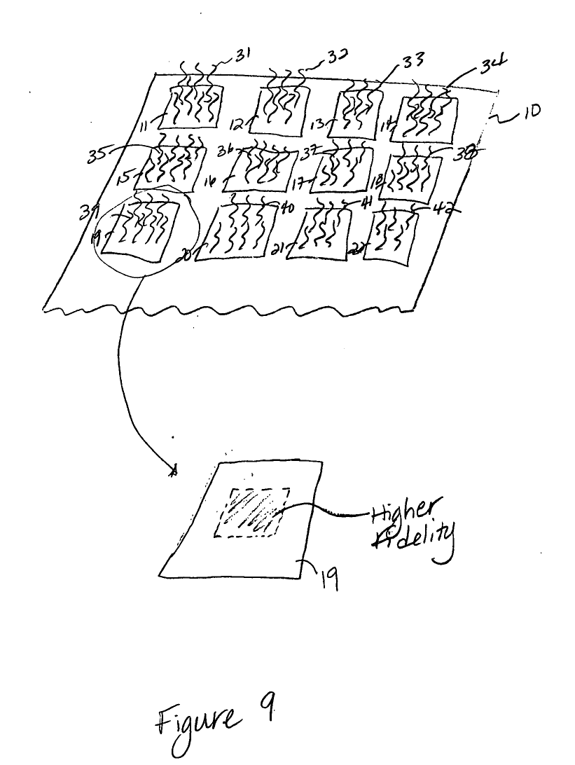

In another aspect, the invention provides methods for assembling polynucleotide constructs that involve one or more error reduction processes. FIG. 7 provides a flow diagram showing an iterative process involving error reduction and/or amplification followed by assembly. In one embodiment, construction oligonucleotides are synthesized and then subjected to one or more rounds of error reduction and/or amplification. For example, the construction oligonucleotides may be subjected to error reduction followed by amplification or amplification followed by error reduction. Successive rounds of amplification and error reduction may be repeated until a desired pool of construction oligonucleotides is obtained. The pool of construction oligonucleotides may then be subjected to assembly. The subassembly products may then be subjected to error reduction followed by amplification or amplification followed by error reduction. Successive rounds of amplification and error reduction may be repeated until a desired pool of subassemblies is obtained. The subassembly pool may represent the final polynucleotide constructs desired. However, in certain embodiments, the subassemblies may become the building blocks for further successive rounds of assembly into even longer polynucleotide constructs. At each stage, one or more rounds of error reduction followed by amplification or amplification followed by error reduction may be carried out until a final desired product having a desired level of fidelity has been obtained. In certain embodiments, it may be desirable to add in a round of denaturation/annealing prior to conducting an error reduction process. This is especially optimal when amplification has been conducted prior to error reduction. As shown in FIG. 8, the denaturation/renaturation process will increase the percent of error laden copies that may be removed by randomly reassociating complementary strands that likely do not contain errors at the same position (e.g., errors that were introduced early in the process and possibly perpetuated during amplification). As discussed in greater detail below, the type of error reduction that is utilized may vary based on the stage of assembly being conducted. For example, in an exemplary embodiment, error filtration by hybridization to selection oligonucleotides may be carried out on a pool of construction oligonucleotides that have not undergone assembly; error filtration using a mismatch binding agent may be carried out on a pool of subassemblies or final polynucleotide constructs having an intermediate length (e.g., from about 1 kb to about 10 kb, or about 1 kb to about 5 kb); and error correction may be carried out on a pool of subassemblies or final polynucleotide constructs having longer lengths (e.g., greater than about 5 kb, 10 kb, 25 kb, 50 kb, 100 kb, 1 megabase, or more). Based on the disclosure herein, one of ordinary skill in the art will be able to conduct the proper sequence of amplification, error reduction and assembly to produce a desired product.

3. Oligonucleotide Design and Synthesis

In various embodiments, the methods described herein utilize construction and/or selection oligonucleotides. The sequences of the construction and/or selection oligonucleotides will be determined based on the sequence of the final polynucleotide construct that is desired to be synthesized. Essentially the sequence of the polynucleotide construct may be divided up into a plurality of overlapping shorter sequences that can then be synthesized in parallel and assembled into the final desired polynucleotide construct using the methods described herein. Design of the construction and/or selection oligonucleotides may be facilitated by the aid of a computer program such as, for example, DNAWorks (Hoover and Lubkowski, Nucleic Acids Res. 30: e43 (2002), Gene2Oligo (Rouillard et al., Nucleic Acids Res. 32: W176-180 (2004) and world wide web at berry.engin.umich.edu/gene2oligo), or the implementation systems and methods discussed further below. In certain embodiments, it may be desirable to design a plurality of construction oligonucleotide/selection oligonucleotide pairs to have substantially similar melting temperatures in order to facilitate manipulation of the plurality of oligonucleotides in a single pool. This process may be facilitated by the computer programs described above. Normalizing melting temperatures between a variety of oligonucleotide sequences may be accomplished by varying the length of the oligonucleotides and/or by codon remapping the sequence (e.g., varying the A/T vs. G/C content in one or more oligonucleotides without altering the sequence of a polynucleotide that may ultimately be encoded thereby) (see e.g., WO 99/58721).

In certain embodiments, the construction oligonucleotides are designed to provide essentially the full complement of sense and antisense strands of the desired polynucleotide construct. For example, the construction oligonucleotides merely need to be hybridized together and subjected to ligation in order to form the full polynucleotide construct. In other embodiments, the complement of construction oligonucleotides may be designed to cover the full sequence, but leave single stranded gaps that may be filed in by chain extension prior to ligation. This embodiment will facilitate production of polynucleotide constructs because it requires synthesis of fewer and/or shorter construction oligonucleotides and/or selection oligonucleotides.

In one embodiment, construction and/or selection oligonucleotides may comprise universal tags. Universal tags are sequences that flank a construction oligonucleotide on either the 5′ end or 3′ end or both and are common to at least a portion of the construction and/or selection oligonucleotides in a pool. Exemplary universal tags may comprise, for example, one or more of the following: a universal primer binding site, a mismatch repair enzyme cut site, a tag for isolation/immobilization of the oligonucleotide, and a restriction endonuclease cleavage site at the junction between the universal tags and the construction oligonucleotide.

In an exemplary embodiment, construction and/or selection oligonucleotides may comprise one or more sets of binding sites for universal primers that may be used for amplification of a pool of nucleic acids with one set, or a few sets, of primers. The sequence of the universal primer binding sites may be chosen to have an appropriate length and sequence to permit efficient primer hybridization and chain extension. Additionally, the sequence of the universal primer binding sites may be optimized so as to minimize non-specific binding to an undesired region of a nucleic acid in the pool. Design of universal primers and binding sites for the universal primers may be facilitated using a computer program such as, for example, DNAWorks (supra), Gene2Oligo (supra), or the implementation systems and methods discussed further below. In certain embodiments, it may be desirable to design several sets of universal primers/primer binding sites that will permit amplification of nucleic acids at different stages of polynucleotide construction (FIG. 6). For example, one set of universal primers may be used to amplify a set of construction and/or selection oligonucleotides. After assembly of a set of construction oligonucleotides into a subassembly, the subassembly may be amplified using the same or a different set of universal primers. For example, the 3′ and 5′ most terminal construction oligonucleotides that are incorporated into the subassembly may contain two or more nested sets of universal primer binding sites, the outermost set which may be used for initial amplification of the construction oligos and second set that may be used to amplify the subassembly. It is possible to incorporate multiple sets of universal primers for amplification at each stage of an assembly (e.g., construction and/or selection oligonucleotides, subassemblies, and/or polynucleotide constructs).

In exemplary embodiments, the universal primers may be designed as temporary primers, e.g., primers that can be removed from the nucleic acid molecule by chemical or enzymatic cleavage. Methods for chemical, thermal, light based, or enzymatic cleavage of nucleic acids are described in detail below. In an exemplary embodiment, the universal primers may be removed using a Type IIS restriction endonuclease or a DNA glycosylase.

Construction and/or selection oligonucleotides may be prepared by any method known in the art for preparation of oligonucleotides having a desired sequence. For example, oligonucleotides may be isolated from natural sources, purchased from commercial sources, or designed from first principals. Preferably, oligonucleotides may be synthesized using a method that permits high-throughput, parallel synthesis so as to reduce cost and production time and increase flexibility. In an exemplary embodiment, construction and/or selection oligonucleotides may be synthesized on a solid support in an array format, e.g., a microarray of single stranded DNA segments synthesized in situ on a common substrate wherein each oligonucleotide is synthesized on a separate feature or location on the substrate. Arrays may be constructed, custom ordered, or purchased from a commercial vendor. Various methods for constructing arrays are well known in the art. For example, methods and techniques applicable to synthesis of construction and/or selection oligonucleotide synthesis on a solid support, e.g., in an array format have been described, for example, in WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752 and Zhou et al., Nucleic Acids Res. 32: 5409-5417 (2004).

In an exemplary embodiment, construction and/or selection oligonucleotides may be synthesized on a solid support using maskless array synthesizer (MAS). Maskless array synthesizers are described, for example, in PCT application No. WO 99/42813 and in corresponding U.S. Pat. No. 6,375,903. Other examples are known of maskless instruments which can fabricate a custom DNA microarray in which each of the features in the array has a single stranded DNA molecule of desired sequence. The preferred type of instrument is the type shown in FIG. 5 of U.S. Patent No. 6,375,903, based on the use of reflective optics. It is a desirable that this type of maskless array synthesizer is under software control. Since the entire process of microarray synthesis can be accomplished in only a few hours, and since suitable software permits the desired DNA sequences to be altered at will, this class of device makes it possible to fabricate microarrays including DNA segments of different sequence every day or even multiple times per day on one instrument. The differences in DNA sequence of the DNA segments in the microarray can also be slight or dramatic, it makes no different to the process. The MAS instrument may be used in the form it would normally be used to make microarrays for hybridization experiments, but it may also be adapted to have features specifically adapted for the compositions, methods, and systems described herein. For example, it may be desirable to substitute a coherent light source, i.e. a laser, for the light source shown in FIG. 5 of the above-mentioned U.S. Pat. No. 6,375,903. If a laser is used as the light source, a beam expanded and scatter plate may be used after the laser to transform the narrow light beam from the laser into a broader light source to illuminate the micromirror arrays used in the maskless array synthesizer. It is also envisioned that changes may be made to the flow cell in which the microarray is synthesized. In particular, it is envisioned that the flow cell can be compartmentalized, with linear rows of array elements being in fluid communication with each other by a common fluid channel, but each channel being separated from adjacent channels associated with neighboring rows of array elements. During microarray synthesis, the channels all receive the same fluids at the same time. After the DNA segments are separated from the substrate, the channels serve to permit the DNA segments from the row of array elements to congregate with each other and begin to self-assemble by hybridization.

Other methods synthesizing construction and/or selection oligonucleotides include, for example, light-directed methods utilizing masks, flow channel methods, spotting methods, pin-based methods, and methods utilizing multiple supports.

Light directed methods utilizing masks (e.g., VLSEPS™ methods) for the synthesis of oligonucleotides is described, for example, in U.S. Pat. Nos. 5,143,854, 5,510,270 and 5,527,681. These methods involve activating predefined regions of a solid support and then contacting the support with a preselected -monomer solution. Selected regions can be activated by irradiation with a light source through a mask much in the manner of photolithography techniques used in integrated circuit fabrication. Other regions of the support remain inactive because illumination is blocked by the mask and they remain chemically protected. Thus, a light pattern defines which regions of the support react with a given monomer. By repeatedly activating different sets of predefined regions and contacting different monomer solutions with the support, a diverse array of polymers is produced on the support. Other steps, such as washing unreacted monomer solution from the support, can be used as necessary. Other applicable methods include mechanical techniques such as those described in U.S. Pat. No. 5,384,261.

Additional methods applicable to synthesis of construction and/or selection oligonucleotides on a single support are described, for example, in U.S. Pat. No. 5,384,261. For example reagents may be delivered to the support by either (1) flowing within a channel defined on predefined regions or (2) “spotting” on predefined regions. Other approaches, as well as combinations of spotting and flowing, may be employed as well. In each instance, certain activated regions of the support are mechanically separated from other regions when the monomer solutions are delivered to the various reaction sites.

Flow channel methods involve, for example, microfluidic systems to control synthesis of oligonucleotides on a solid support. For example, diverse polymer sequences may be synthesized at selected regions of a solid support by forming flow channels on a surface of the support through which appropriate reagents flow or in which appropriate reagents are placed. One of skill in the art will recognize that there are alternative methods of forming channels or otherwise protecting a portion of the surface of the support. For example, a protective coating such as a hydrophilic or hydrophobic coating (depending upon the nature of the solvent) is utilized over portions of the support to be protected, sometimes in combination with materials that facilitate wetting by the reactant solution in other regions. In this manner, the flowing solutions are further prevented from passing outside of their designated flow paths.

Spotting methods for preparation of oligonucleotides on a solid support involve delivering reactants in relatively small quantities by directly depositing them in selected regions. In some steps, the entire support surface can be sprayed or otherwise coated with a solution, if it is more efficient to do so. Precisely measured aliquots of monomer solutions may be deposited dropwise by a dispenser that moves from region to region. Typical dispensers include a micropipette to deliver the monomer solution to the support and a robotic system to control the position of the micropipette with respect to the support, or an ink-jet printer. In other embodiments, the dispenser includes a series of tubes, a manifold, an array of pipettes, or the like so that various reagents can be delivered to the reaction regions simultaneously.

Pin-based methods for synthesis of oligonucleotides on a solid support are described, for example, in U.S. Pat No.5,288,514. Pin-based methods utilize a support having a plurality of pins or other extensions. The pins are each inserted simultaneously into individual reagent containers in a tray. An array of 96 pins is commonly utilized with a 96-container tray, such as a 96-well microtitre dish. Each tray is filled with a particular reagent for coupling in a particular chemical reaction on an individual pin. Accordingly, the trays will often contain different reagents. Since the chemical reactions have been optimized such that each of the reactions can be performed under a relatively similar set of reaction conditions, it becomes possible to conduct multiple chemical coupling steps simultaneously.

In yet another embodiment, a plurality of construction and/or selection oligonucleotides may be synthesized on multiple supports. On example is a bead based synthesis method which is described, for example, in U.S. Pat. Nos. 5,770,358, 5,639,603, and 5,541,061. For the synthesis of molecules such as oligonucleotides on beads, a large plurality of beads are suspended in a suitable carrier (such as water) in a container. The beads are provided with optional spacer molecules having an active site to which is complexed, optionally, a protecting group. At each step of the synthesis, the beads are divided for coupling into a plurality of containers. After the nascent oligonucleotide chains are deprotected, a different monomer solution is added to each container, so that on all beads in a given container, the same nucleotide addition reaction occurs. The beads are then washed of excess reagents, pooled in a single container, mixed and re-distributed into another plurality of containers in preparation for the next round of synthesis. It should be noted that by virtue of the large number of beads utilized at the outset, there will similarly be a large number of beads randomly dispersed in the container, each having a unique oligonucleotide sequence synthesized on a surface thereof after numerous rounds of randomized addition of bases. An individual bead may be tagged with a sequence which is unique to the double-stranded oligonucleotide thereon, to allow for identification during use.

Various exemplary protecting groups useful for synthesis of oligonucleotides on a solid support are described in, for example, Atherton et al., 1989, Solid Phase Peptide Synthesis, IRL Press.

In various embodiments, the methods described herein utilize solid supports for immobilization of nucleic acids. For example, oligonucleotides may be synthesized on one or more solid supports. Additionally, selection oligonucleotides may be immobilized on a solid support to facilitate removal of construction oligonucleotides containing sequence errors. Exemplary solid supports include, for example, slides, beads, chips, particles, strands, gels, sheets, tubing, spheres, containers, capillaries, pads, slices, films, or plates. In various embodiments, the solid supports may be biological, nonbiological, organic, inorganic, or combinations thereof. When using supports that are substantially planar, the support may be physically separated into regions, for example, with trenches, grooves, wells, or chemical barriers (e.g., hydrophobic coatings, etc.). Supports that are transparent to light are useful when the assay involves optical detection (see e.g., U.S. Pat. No. 5,545,531). The surface of the solid support will typically contain reactive groups, such as carboxyl, amino, and hydroxyl or may be coated with functionalized silicon compounds (see e.g., U.S. Pat. No. 5,919,523).

In one embodiment, the oligonucleotides synthesized on the solid support may be used as a template for the production of construction oligonucleotides and/or selection oligonucleotides for assembly into longer polynucleotide constructs. For example, the support bound oligonucleotides may be contacted with primers that hybridize to the oligonucleotides under conditions that permit chain extension of the primers. The support bound duplexes may then be denatured and subjected to further rounds of amplification.

In another embodiment, the support bound oligonucleotides may be removed from the solid support prior to assembly into polynucleotide constructs. The oligonucleotides may be removed from the solid support, for example, by exposure to conditions such as acid, base, oxidation, reduction, heat, light, metal ion catalysis, displacement or elimination chemistry, or by enzymatic cleavage.

In one embodiment, oligonucleotides may be attached to a solid support through a cleavable linkage moiety. For example, the solid support may be functionalized to provide cleavable linkers for covalent attachment to the oligonucleotides. The linker moiety may be of six or more atoms in length. Alternatively, the cleavable moiety may be within an oligonucleotide and may be introduced during in situ synthesis. A broad variety of cleavable moieties are available in the art of solid phase and microarray oligonucleotide synthesis (see e.g., Pon, R., Methods Mol. Biol. 20:465-496 (1993); Verma et al., Annu. Rev. Biochem. 67:99-134 (1998); U.S. Pat. Nos. 5,739,386, 5,700,642 and 5,830,655; and U.S. Patent Publication Nos. 2003/0186226 and 2004/0106728). A suitable cleavable moiety may be selected to be compatible with the nature of the protecting group of the nucleoside bases, the choice of solid support, and/or the mode of reagent delivery, among others. In an exemplary embodiment, the oligonucleotides cleaved from the solid support contain a free 3′-OH end. Alternatively, the free 3′-OH end may also be obtained by chemical or enzymatic treatment, following the cleavage of oligonucleotides. The cleavable moiety may be removed under conditions which do not degrade the oligonucleotides. Preferably the linker may be cleaved using two approaches, either (a) simultaneously under the same conditions as the deprotection step or (b) subsequently utilizing a different condition or reagent for linker cleavage after the completion of the deprotection step.

The covalent immobilization site may either be at the 5′ end of the oligonucleotide or at the 3′end of the oligonucleotide. In some instances, the immobilization site may be within the oligonucleotide (i.e. at a site other than the 5′ or 3′ end of the oligonucleotide). The cleavable site may be located along the oligonucleotide backbone, for example, a modified 3′-5′ intemucleotide linkage in place of one of the phosphodiester groups, such as ribose, dialkoxysilane, phosphorothioate, and phosphoramidate intemucleotide linkage. The cleavable oligonucleotide analogs may also include a substituent on, or replacement of, one of the bases or sugars, such as 7-deazaguanosine, 5-methylcytosine, inosine, uridine, and the like.

In one embodiment, cleavable sites contained within the modified oligonucleotide may include chemically cleavable groups, such as dialkoxysilane, 3′-(S)-phosphorothioate, 5′-(S)-phosphorothioate, 3′-(N)-phosphoramidate, 5′-(N)phosphoramidate, and ribose. Synthesis and cleavage conditions of chemically cleavable oligonucleotides are described in U.S. Pat. Nos. 5,700,642 and 5,830,655. For example, depending upon the choice of cleavable site to be introduced, either a functionalized nucleoside or a modified nucleoside dimer may be first prepared, and then selectively introduced into a growing oligonucleotide fragment during the course of oligonucleotide synthesis. Selective cleavage of the dialkoxysilane may be effected by treatment with fluoride ion. Phosphorothioate intemucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide.

In another embodiment, a non-cleavable hydroxyl linker may be converted into a cleavable linker by coupling a special phosphoramidite to the hydroxyl group prior to the phosphoramidite or H-phosphonate oligonucleotide synthesis as described in U.S. Patent Application Publication No. 2003/0186226. The cleavage of the chemical phosphorylation agent at the completion of the oligonucleotide synthesis yields an oligonucleotide bearing a phosphate group at the 3′ end. The 3′-phosphate end may be converted to a 3′ hydroxyl end by a treatment with a chemical or an enzyme, such as alkaline phosphatase, which is routinely carried out by those skilled in the art.

In another embodiment, the cleavable linking moiety may be a TOPS (two oligonucleotides per synthesis) linker (see e.g., PCT publication WO 93/20092). For example, the TOPS phosphoramidite may be used to convert a non-cleavable hydroxyl group on the solid support to a cleavable linker. A preferred embodiment of TOPS reagents is the Universal TOPS™ phosphoramidite. Conditions for Universal TOPS™ phosphoramidite preparation, coupling and cleavage are detailed, for example, in Hardy et al, Nucleic Acids Research 22(15):2998-3004 (1994). The Universal TOPS™ phosphoramidite yields a cyclic 3′ phosphate that may be removed under basic conditions, such as the extended ammonia and/or ammonia/methylamine treatment, resulting in the natural 3′ hydroxy oligonucleotide.

In another embodiment, a cleavable linking moiety may be an amino linker. The resulting oligonucleotides bound to the linker via a phosphoramidite linkage may be cleaved with 80% acetic acid yielding a 3′-phosphorylated oligonucleotide.

In another embodiment, the cleavable linking moiety may be a photocleavable linker, such as an ortho-nitrobenzyl photocleavable linker. Synthesis and cleavage conditions of photolabile oligonucleotides on solid supports are described, for example, in Venkatesan et al. J. of Org. Chem. 61:525-529 (1996), Kahl et al., J. of Org. Chem. 64:507-510 (1999), Kahl et al., J. of Org. Chem. 63:4870-4871 (1998), Greenberg et al., J. of Org. Chem. 59:746-753 (1994), Holmes et al., J. of Org. Chem. 62:2370-2380 (1997), and U.S. Pat. No. 5,739,386. Ortho-nitobenzyl-based linkers, such as hydroxymethyl, hydroxyethyl, and Fmoc-aminoethyl carboxylic acid linkers, may also be obtained commercially.

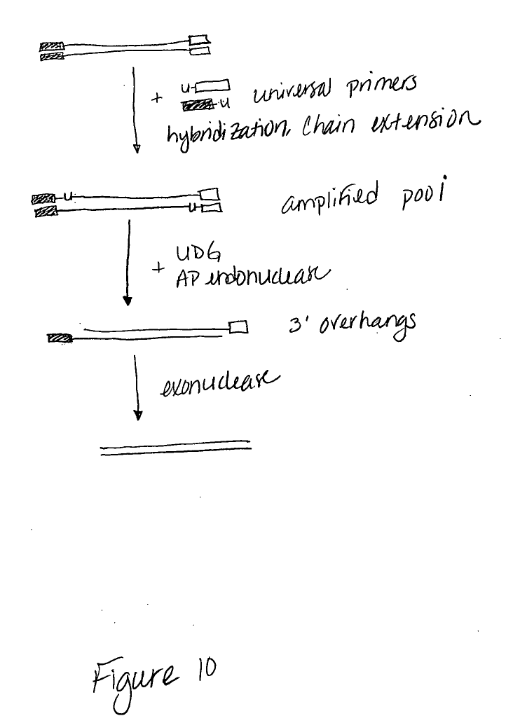

When synthesizing oligonucleotides on a solid support, the oligonucleotides at the edge of a particular location on the support tend to have a higher percentage of errors than the oligonucleotides located toward the center of that position. For example, FIG. 9 shows an illustration of a solid support containing locations 11-22 each having a different oligonucleotide sequence (e.g., 31-42) that has been synthesized on the different locations. As shown in the detailed view of location 19, the shaded region in the center represents the portion of the location that produces oligonucleotides having relatively higher fidelity (e.g., less sequence errors) as compared to oligonucleotides synthesized at the edges of the location. To increase the fidelity of the starting pool of construction and/or selection oligonucleotides it may be desirable to selectively release the oligonucleotides located toward the center of a location and minimize the oligonucleotides released from near the edges of a location. This may be accomplished using photolabile linking moieties for attachment of the oligonucleotides to the solid support. The oligonucleotides towards the center of the location may then be selectively removed by directing light to the center of the location. Highly accurate irradiation of the center of a location on a solid support may be achieved, for example, using a maskless array synthesizer or MAS (see e.g., PCT Publication W099/42813 and U.S. Pat. No. 6,375,903). The MAS instrument may be used in the form it would normally be used to make microarrays for hybridization experiments, but it may also be adapted to have features specifically adapted for this application. For example, it may be desirable to use a coherent light source, i.e. a laser, to provide a narrow light beam and thus more accurate control over location of cleavage of the oligonucleotides.

In another embodiment, shorter construction oligonucleotides may be synthesized and used for construction because shorter oligonucleotides should be more pure and contain fewer sequence errors than longer oligonucleotides. For example, construction oligonucleotides may be from about 30 to about 100 nucleotides, from about 30 to about 75 nucleotides, or from about 30 to about 50 oligonucleotides. In other embodiments, the construction oligonucleotides are sufficient to essentially cover the entire sequence of the synthetic polynucleotide (e.g., there are no gaps between the oligonucleotides that need to be filled in by polymerase). The oligonucleotides themselves may serve as a checking mechanism because mismatched oligonucleotides will anneal less preferentially than fully matched oligonucleotides and therefore errors containing sequences may be reduced by carefully controlling hybridization conditions.

In another embodiment, oligonucleotides may be removed from a solid support by an enzyme such as nucleases and/or glycosylases. A wide range of oligonucleotide bases, e.g. uracil, may be removed by a DNA glycosylase which cleaves the N-glycosylic bond between the base and deoxyribose, thus leaving an abasic site (Krokan et. al., Biochem. J. 325:1-16 (1997)). The abasic site in an oligonucleotide may then be cleaved by an AP endonuclease such as Endonuclease IV, leaving a free 3′-OH end. In another embodiment, oligonucleotides may be removed from a solid support upon exposure to one or more restriction endonucleases, including, for example, class IIs restriction enzymes. For example, a restriction endonuclease recognition sequence may be incorporated into the immobilized oligonucleotides and the oligonucleotides may be contacted with one or more restriction endonucleases to remove the oligonucleotides from the support. In various embodiments, when using enzymatic cleavage to remove the oligonucleotides from the support, it may be desirable to contact the single stranded immobilized oligonucleotides with primers, polymerase and dNTPs to form immobilized duplexes. The duplexes may then be contacted with the enzyme (e.g., restriction endonuclease, DNA glycosylase, etc.) to remove the duplexes from the surface of the support. Methods for synthesizing a second strand on a support bound oligonucleotide and methods for enzymatic removal of support bound duplexes are described, for example, in U.S. Pat. No. 6,326,489. Alternatively, short oligonucleotides that are complementary to the restriction endonuclease recognition and/or cleavage site (e.g., but are not complementary to the entire support bound oligonucleotide) may be added to the support bound oligonucleotides under hybridization conditions to facilitate cleavage by a restriction endonuclease (see e.g., PCT Publication No. WO 04/024886).

4. Amplification of Nucleic Acids

In various embodiments, the methods disclosed herein comprise amplification of nucleic acids including, for example, construction oligonucleotides, selection oligonucleotides, subassemblies and/or polynucleotide constructs. Amplification may be carried out at one or more stages during an assembly scheme and/or may be carried out one or more times at a given stage during assembly. Amplification methods may comprise contacting a nucleic acid with one or more primers that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, or any other nucleic acid amplification method using techniques well known to those of skill in the art. In exemplary embodiments, the methods disclosed herein utilize PCR amplification.

In certain embodiments, a primer set specific for a nucleic acid sequence may be used to amplify a specific nucleic acid sequence that is isolated or to amplify a specific nucleic acid sequence that is part of a pool of nucleic acid sequences. In another embodiment, a plurality of primer sets may be used to amplify a plurality of specific nucleic acid sequences that may optionally be pooled together into a single reaction mixture. In an exemplary embodiment, a set of universal primers may be used to amplify a plurality of nucleic acid sequences that may be in a single pool or separated into a plurality of pools (FIG. 5). When amplifying nucleic acids at different stages during assembly it may be desirable to utilize a different set of universal primers for each stage at which amplification is desired (FIG. 6). For example, a first set of universal primers may be used to amplify construction and/or selection oligonucleotides and a second set of universal primers may be used to amplify a subassembly or polynucleotide construct (FIG. 6). As described above, the construction oligonucleotides and/or selection oligonucleotides may be designed with primer binding sites for one or more sets of universal primers. Alternatively, primer binding sites may be added to a nucleic acid after synthesis through the use of chimeric primers that contain a region complementary to the target nucleic acid and a non-complementary region that becomes incorporated during the amplification process (see e.g., WO 99/58721).

In exemplary embodiments, primers/primer binding sites may be designed to be temporary, e.g., to permit removal of the primers/primer binding sites at a desired stage during assembly. Temporary primers may be designed so as to be removable by chemical, thermal, light based, or enzymatic cleavage. Cleavage may occur upon addition of an external factor (e.g., an enzyme, chemical, heat, light, etc.) or may occur automatically after a certain time period (e.g., after n rounds of amplification). In one embodiment, temporary primers may be removed by chemical cleavage. For example, primers having acid labile or base labile sites may be used for amplification. The amplified pool may then be exposed to acid or base to remove the primer/primer binding sites at the desired location. Alternatively, the temporary primers may be removed by exposure to heat and/or light. For example, primers having heat labile or photolabile sites may be used for amplification. The amplified pool may then be exposed to heat and/or light to remove the primer/primer binding sites at the desired location. In another embodiment, an RNA primer may be used for amplification thereby forming short stretches of RNA/DNA hybrids at the ends of the nucleic acid molecule. The primer site may then be removed by exposure to an RNase (e.g., RNase H). In various embodiments, the method for removing the primer may only cleave a single strand of the amplified duplex thereby leaving 3′ or 5′ overhangs. Such overhangs may be removed using an exonuclease to form blunt ended double stranded duplexes. For example, RecJ_{f} may be used to remove single stranded 5′ overhangs and Exonuclease I or Exonuclease T may be used to remove single stranded 3′ overhangs. Additionally, S₁ nuclease, P₁ nuclease, mung bean nuclease, and CEL I nuclease, may be used to remove single stranded regions from a nucleic acid molecule. RecJ_{f}, Exonuclease I, Exonuclease T, and mung bean nuclease are commercially available, for example, from New England Biolabs (Beverly, Mass.). S1 nuclease, P1 nuclease and CEL I nuclease are described, for example, in Vogt, V. M., Eur. J. biochem., 33: 192-200 (1973); Fujimoto et al., Agric. Biol. Chem. 38: 777-783 (1974); Vogt, V. M., Methods Enzymol. 65: 248-255 (1980); and Yang et al., Biochemistry 39: 3533-3541 (2000).

In one embodiment, the temporary primers may be removed from a nucleic acid by chemical, thermal, or light based cleavage. Exemplary chemically cleavable internucleotide linkages for use in the methods described herein include, for example, β-cyano ether, 5′-deoxy-5′-aminocarbamate, 3′deoxy-3′-aminocarbamate, urea, 2′cyano-3′, 5′-phosphodiester, 3′-(S)-phosphorothioate, 5′-(S)-phosphorothioate, 3′-(N)-phosphoramidate, 5′-(N)-phosphoramidate, α-amino amide, vicinal diol, ribonucleoside insertion, 2′-amino-3′,5′-phosphodiester, allylic sulfoxide, ester, silyl ether, dithioacetal, 5′-thio-furmal, α-hydroxy-methyl-phosphonic bisamide, acetal, 3′-thio-furmal, methylphosphonate and phosphotriester. Intemucleoside silyl groups such as trialkylsilyl ether and dialkoxysilane are cleaved by treatment with fluoride ion. Base-cleavable sites include β-cyano ether, 5′-deoxy-5′-aminocarbamate, 3′-deoxy-3′-aminocarbamate, urea, 2′-cyano-3′, 5′-phosphodiester, 2′-amino-3′, 5′-phosphodiester, ester and ribose. Thio-containing internucleotide bonds such as 3′-(S)-phosphorothioate and 5′-(S)-phosphorothioate are cleaved by treatment with silver nitrate or mercuric chloride. Acid cleavable sites include 3′-(N)-phosphoramidate, 5′-(N)-phosphoramidate, dithioacetal, acetal and phosphonic bisamide. An α-aminoamide internucleoside bond is cleavable by treatment with isothiocyanate, and titanium may be used to cleave a 2′-amino-3′,5′-phosphodiester-O-ortho-benzyl internucleoside bond. Vicinal diol linkages are cleavable by treatment with periodate. Thermally cleavable groups include allylic sulfoxide and cyclohexene while photo-labile linkages include nitrobenzylether and thymidine dimer. Methods synthesizing and cleaving nucleic acids containing chemically cleavable, thermally cleavable, and photo-labile groups are described for example, in U.S. Pat. No. 5,700,642.

In other embodiments, temporary primers/primer binding sites may be removed using enzymatic cleavage. For example, primers/primer binding sites may be designed to include a restriction endonuclease cleavage site. After amplification, the pool of nucleic acids may be contacted with one or more endonucleases to produce double stranded breaks thereby removing the primers/primer binding sites. In certain embodiments, the forward and reverse primers may be removed by the same or different restriction endonucleases. Any type of restriction endonuclease may be used to remove the primers/primer binding sites from nucleic acid sequences. A wide variety of restriction endonucleases having specific binding and/or cleavage sites are commercially available, for example, from New England Biolabs (Beverly, Mass.). In various embodiments, restriction endonucleases that produce 3′ overhangs, 5′ overhangs or blunt ends may be used. When using a restriction endonuclease that produces an overhang, an exonuclease (e.g., RecJ_{f}, Exonuclease I, Exonuclease T, S₁ nuclease, P₁ nuclease, mung bean nuclease, CEL I nuclease, etc.) may be used to produce blunt ends. Alternatively, the sticky ends formed by the specific restriction endonuclease may be used to facilitate assembly of subassemblies in a desired arrangement (see e.g., FIG. 4A). In an exemplary embodiment, a primer/primer binding site that contains a binding and/or cleavage site for a type IIS restriction endonuclease may be used to remove the temporary primer.

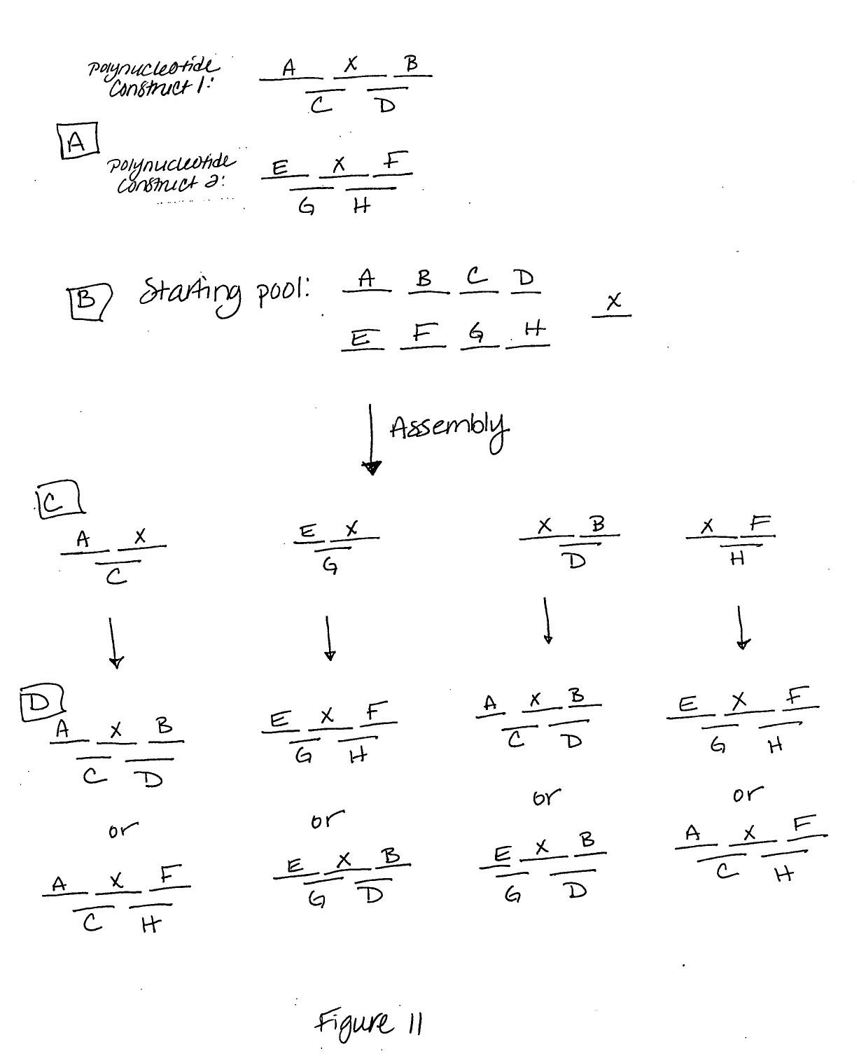

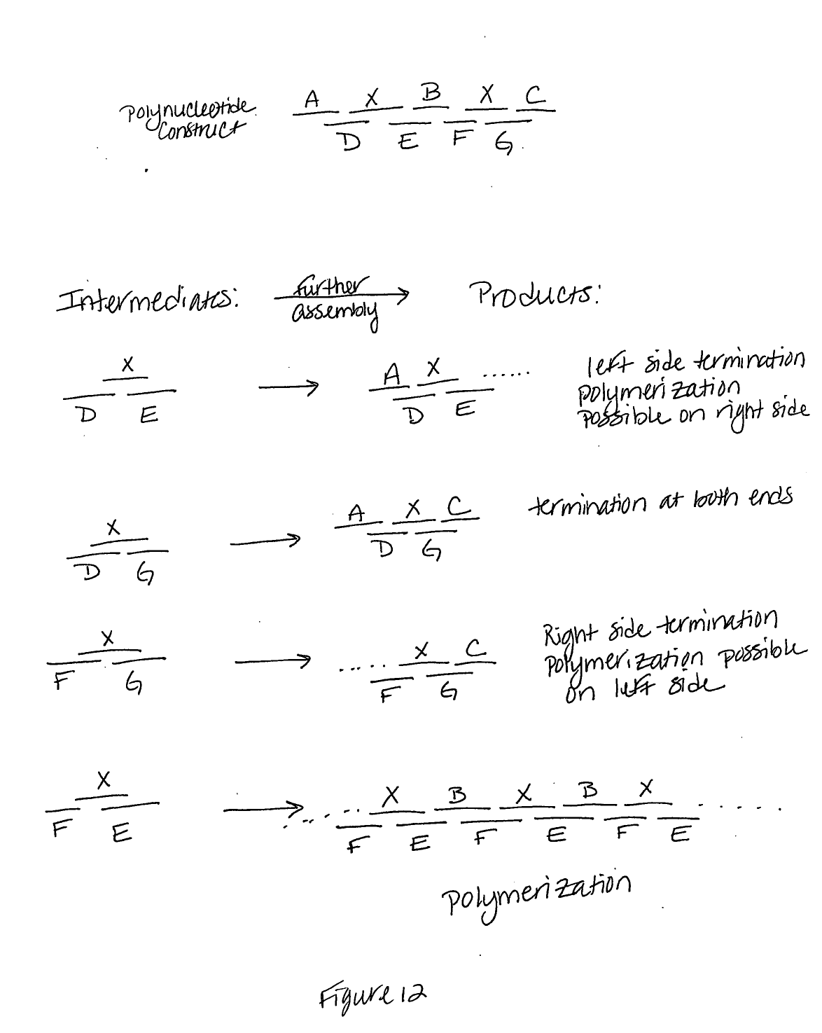

In an exemplary embodiment, a temporary primer may be designed to be removed using uracil DNA glycosylase and an AP endonuclease. For example, a primer may be designed to contain one or more uracil residues at the desired site of cleavage. During amplification, each amplified strand will incorporate the uracil residues at the desired location. The amplified pool may then be contacted with uracil DNA glycosylase (which will remove the uracil base from the backbone) and an AP endonuclease (which will cleave the backbone at the abasic site causing a single stranded break) producing a duplex having 3′ overhangs at each end. The overhangs may be removed using an exonuclease such as, for example, Exonuclease I or Exonuclease T, thereby forming a blunt ended double stranded duplex. This is illustrated in FIG. 10. In various other embodiments, other combinations of bases and DNA glycosylases may be used as means to remove a primer/primer binding site, including for example, Hmu-DNA glycosylase (recognizes hydroxymethyl uracil), 5-mC-DNA glycosylase (recognizes 5-methylcytosine), Hx-DNA glycosylase (recognizes hypoxanthine), 3-mA-DNA-glycosylase I (recognizes 3-methyladenine), 3-mA-DNA-glycosylase II (recognizes 3-methyladenine, 7-methylguanine and 3-methylguanine), FaPy-DNA glycosylase (recognizes formamidopyrimidines and 8 hydroxyguanine), and 5,6-HT-DNA-glycosylase (recognizes 5,6 hydrated thymines).

Primers suitable for use in the amplification methods disclosed herein may be designed with the aid of a computer program, such as, for example, DNAWorks (supra), Gene2oligo (supra), or the implementation systems and methods discussed further below. Typically primers are from about 5 to about 500, about 10 to about 100, about 10 to about 50, or about 10 to about 30 nucleotides in length. In exemplary embodiments, a set of primers or a plurality of sets of primers may be designed so as to have substantially similar melting temperatures to facilitate manipulation of a complex reaction mixture.

The melting temperature may be influenced, for example, by primer length and nucleotide composition.

In certain embodiments, it may be desirable to utilize a primer comprising one or more modifications such as a cap (e.g., to prevent exonuclease cleavage), a linking moiety (such as those described above to facilitate immobilization of an oligonucleotide onto a substrate), or a label (e.g., to facilitate detection, isolation and/or immobilization of a nucleic acid construct). Suitable modifications include, for example, various enzymes, prosthetic groups, luminescent markers, bioluminescent markers, fluorescent markers (e.g., fluorescein), radiolabels (e.g., ³²P, ³⁵S, etc.), biotin, polypeptide epitopes, etc. Based on the disclosure herein, one of skill in the art will be able to select an appropriate primer modification for a given application.

5. Assembly Methods

In various embodiments, the methods disclosed herein utilize methods for assembling long polynucleotide constructs from shorter oligonucleotides including, for example, PCR based assembly methods (including PAM or polymerase assembly multiplexing) and ligation based assembly methods (e.g., joining of polynucleotide segments having cohesive ends). In an exemplary embodiment, a plurality of polynucleotide constructs may be assembled in a single reaction mixture. In other embodiments, hierarchical based assembly methods may be used, for example, when synthesizing a large number of polynucleotide constructs, when synthesizing a polynucleotide construct that contains a region of internal homology, or when synthesizing two or more polynucleotide constructs that are highly homologous or contain regions of homology.

In one embodiment, assembly PCR may be used in accordance with the methods described herein. Assembly PCR uses polymerase-mediated chain extension in combination with at least two polynucleotides having complementary ends which can anneal such that at least one of the polynucleotides has a free 3′-hydroxyl capable of polynucleotide chain elongation by a polymerase (e.g., a thermostable polymerase (e.g., Taq polyrnerase, VENT™ polymerase (New England Biolabs), TthI polymerase (Perkin-Elmer) and the like). Overlapping oligonucleotides may be mixed in a standard PCR reaction containing dNTPs, a polymerase, and buffer. The overlapping ends of the oligonucleotides, upon annealing, create regions of double-stranded nucleic acid sequences that serve as primers for the elongation by polymerase in a PCR reaction. Products of the elongation reaction serve as substrates for formation of a longer double-strand nucleic acid sequences, eventually resulting in the synthesis of full-length target sequence (see e.g., FIG. 3B). The PCR conditions may be optimized to increase the yield of the target long DNA sequence.

In certain embodiments, the target sequence may be obtained in a single step by mixing together all of the overlapping oligonucleotides needed to form the polynucleotide construct of interest. Alternatively, a series of PCR reactions may be performed in parallel or serially, such that larger polynucleotide constructs may be assembled from a series of separate PCR reactions whose products are mixed and subjected to a second round of PCR. Moreover, if the self-priming PCR fails to give a full-sized product from a single reaction, the assembly may be rescued by separately PCR-amplifying pairs of overlapping oligonucleotides, or smaller sections of the target nucleic acid sequence, or by conventional filling-in and ligation methods.

Methods for performing assembly PCR are described, for example, in Kodumal et al. (2004) Proc. Natl. Acad. Sci. U.S.A. 101:15573; Stemmer et al. (1995) Gene 164:49; Dillon et al. (1990) BioTechniques 9:298; Hayashi et al. (1994) BioTechniques 17:310; Chen et al. (1994) J. Am. Chem. Soc. 116:8799; Prodromou et al. (1992) Protein Eng. 5:827; U.S. Pat. Nos. 5,928,905 and 5,834,252; and U.S. Patent Application Publication Nos. 2003/0068643 and 2003/0186226.

In an exemplary embodiment, polymerase assembly multiplexing (PAM) may be used to assemble polynucleotide constructs in accordance with the methods described herein (see e.g., Tian et al. (2004) Nature 432:1050; Zhou et al. (2004) Nucleic Acids Res. 32:5409; and Richmond et al. (2004) Nucleic Acids Res. 32:5011). Polymerase assembly multiplexing involves mixing sets of overlapping oligonucleotides and/or amplification primers under conditions that favor sequence-specific hybridization and chain extension by polymerase using the hybridizing strand as a template. The double stranded extension products may optionally be denatured and used for further rounds of assembly until a desired polynucleotide construct has been synthesized.

In various embodiments, methods for assembling polynucleotide constructs in accordance with the methods described herein include, for example, ligation of preformed duplexes (see e.g., Scarpulla et al., Anal. Biochem. 121: 356-365 (1982); Gupta et al., Proc. Natl. Acad. Sci. USA 60: 1338-1344 (1968)), the Fok I method (see e.g., Mandecki and Bolling, Gene 68: 101-107 (1988)), dual asymmetrical PCR (DA-PCR) (see e.g., Stemmer et al., Gene 164: 49-53 (1995); Sandhu et al., Biotechniques 12: 14-16 (1992); Smith et al., Proc. Natl. Acad. Sci. USA 100: 15440-15445 (2003)), overlap extension PCR (OE-PCR) (see e.g., Mehta and Singh, Biotechniques 26: 1082-1086 (1999)), DA-PCR/OE-PCR combination (see e.g., Young and Dong, Nucleic Acids Res. 32: e59 (2004)).

In another embodiment, a combinatorial assembly strategy may be used for assembly of polynucleotides (see e.g., U.S. Pat. Nos. 6,670,127, 6,521,427 and 6,521,427). Briefly, oligonucleotides may be jointly co-annealed by temperature-based slow annealing followed by ligation chain reaction steps using a new oligonucleotide addition with each step. The first oligonucleotide in the chain is attached to a support. The second, overlapping oligonucleotide from the opposite strand is added, annealed and ligated. The third, overlapping oligonucleotide is added, annealed and ligated, and so forth. This procedure is replicated until all oligonucleotides of interest are annealed and ligated. This procedure can be carried out for long sequences using an automated device. The double-stranded nucleic acid sequence is then removed from the solid support.

In certain embodiments, assembly may be facilitated by functional selection of the assembled products in cells. For example, construction oligonucleotides may be assembled into subassemblies using one or more of the PCR based assembly methods described above. The subassemblies may then be cloned into vectors that will facilitate further assembly using ligation by selection (LBS) (see e.g., Kodumal et al., Proc. Nati. Acad. Sci. USA 101: 15573-15578 (2004)). The subassemblies may be cloned into vectors containing a set of unique selective markers using standard recombinant techniques (e.g., restriction enzyme digestion followed by ligation) or using uracil DNA glucosidase/ligation independent cloning (UDG/LIC cloning) (see e.g., Rashtchian, et al., Anal. Biochem. 206: 91-97 (1992); Chanbers et al., Nat. Biotechnol. 21: 1088-1092 (2003); Smith et al., PCR Methods Appl. 2: 328-332 (1993); and Kodumal et al., Proc. Nati. Acad. Sci. USA 101: 15573-15578 (2004)). Two subassemblies in different vectors having unique sets of selection markers may then be cleaved with a set of restriction enzymes, mixed and ligated together. The proper joining of the subassemblies into a desired product may be selected by transforming the products into cells and selecting for the unique combination of markers associated with the desired product. These products may then be optionally subjected to further rounds of assembly by LBS. Such LBS techniques may be carried out in a high throughput, parallel fashion to permit efficient assembly of long nucleic acids. Additionally, subassemblies or products of LBS may be further assembled using traditional recombinant cloning techniques involving restriction endonuclease cleavage, ligation, transformation into cells and growth selection (see e.g., Kodumal et al., Proc. Natl. Acad. Sci. USA 101: 15573-15578 (2004)).

In other embodiments, synthesis of long polynucleotide constructs may be conducted using homologous recombination, site-specific recombination (e.g., using a viral integrase), or transposition. For example, the ends of two or more polynucleotide sequences may be designed to contain sequences specifically designed to facilitate joining of the polynucleotides. Such recombination processes may be carried out in vitro or in vivo (e.g., in a host cell).

In certain embodiments, hierarchical assembly strategies may be used in accordance with the methods disclosed herein. Hierarchical assembly strategies include various methods for controlled mixing of various components of a reaction mixture so as to control the assembly in a staged or stepwise manner (see e.g., U.S. Pat. No. 6,586,211; U.S. Patent Application Publication No. 2004/0166567; PCT Publication No. WO 02/095073; Zhou et al. (2004) Nucleic Acids Res. 32:5409). For example, a plurality of assembly reactions may be conducted in separate pools. Products from these assemblies may then be mixed to together to form even larger assembled products, etc. Alternatively, hierarchical assembly strategies may involve a single reaction mixture that permits external control by varying the reactive species in the mixture. For example, oligonucleotides attached to a solid support via a photolabile linker may be released from the support in a highly specific and controlled manner that can be used to facilitate ordered assembly (e.g., oligonucleotides may be removed from a single addressable location on a solid support in a controlled fashion). A first set of construction oligonucleotides may be released from the support and subjected to assembly. Subsequently a second set of construction oligonucleotides may be released from the support and assembled, etc. In one embodiment, positive and negative strands of construction oligonucleotides may be synthesized on different locations or on different supports. The positive and negative strands may then be released from the chips into separate pools and mixed in a controlled fashion. In another embodiment, hierarchical assembly may be controlled by proximity of construction oligonucleotides on a solid support. For example, two construction oligonucleotides having complementary regions may be synthesized in close proximity to each other. Upon release from the solid support, oligonucleotides located in close proximity to each other will favorably interact due to the higher local concentrations of the oligonucleotides. In an exemplary embodiment, two or more construction oligonucleotides may be synthesized at the same location on a solid support thereby facilitating their interaction (see e.g., U.S. Patent Publication No. 2004/0101894). In yet another embodiment, microfluidic systems may be employed to control the reaction mixture and facilitate the assembly process. For example, oligonucleotides may be synthesized in a flow cell containing channels such that the features of the array are aligned in linear rows which are physically separated from one another thus separate, linear channels in which fluids may flow. Oligonucleotides in a given channel may hybridize with interact with other oligonucleotides in the same channel but will not be exposed to oligonucleotides from other channels. When adjoining oligonucleotide sequences are synthesized in the same channel, they can hybridize to one another after cleavage from the array to form “sub-assemblies”. Various sub-assemblies may then be contacted with other sub-assemblies in order to hybridize larger nucleic acid sequences. Ligases and/or polymerases may be added as needed to fill in and/or join gaps in the nucleic acid sequences.

In yet another embodiment, hierarchical assembly may be carried out using restriction endonucleases to form cohesive ends that may be joined together in a desired order. The construction oligonucleotides may be designed and synthesized to contain recognition and cleavage sites for one or more restriction endonucleases at sites that would facilitate joining in a specified order. After forming DNA duplexes, the pool of oligonucleotides may be contacted with one or more restriction endonucleases to form the cohesive ends. The pool is then exposed to hybridization and ligation conditions to join the duplexes together. The order of joining will be determined by hybridization of the complementary cohesive ends. The restriction endonucleases may be added in a staggered fashion so as to form only a subset of cohesive ends at a time. These ends may then be joined together followed by another round of endonuclease digestion, hybridization, ligation, etc. In an exemplary embodiment, a type IIS endonuclease recognition site may be incorporated into the termini of the construction oligonucleotides to permit cleavage by a type IIS restriction endonuclease.

6. Multiplex Assembly of Homologous Sequences

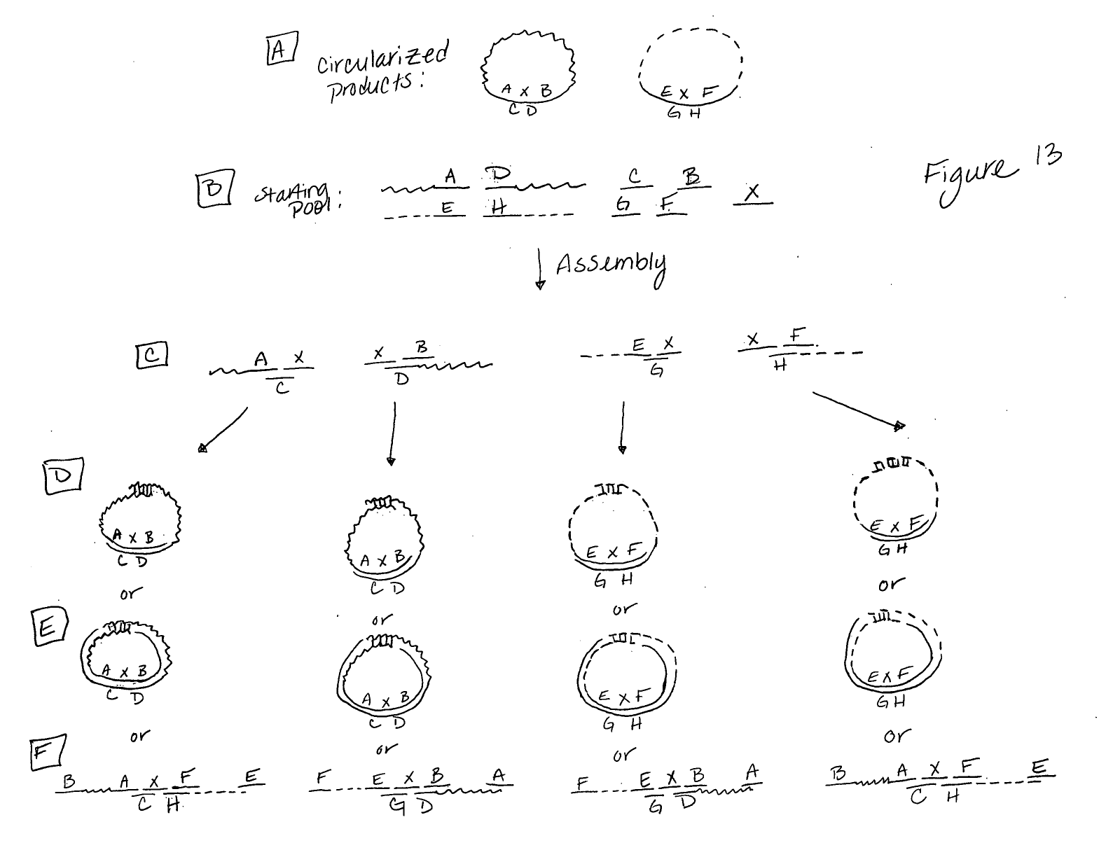

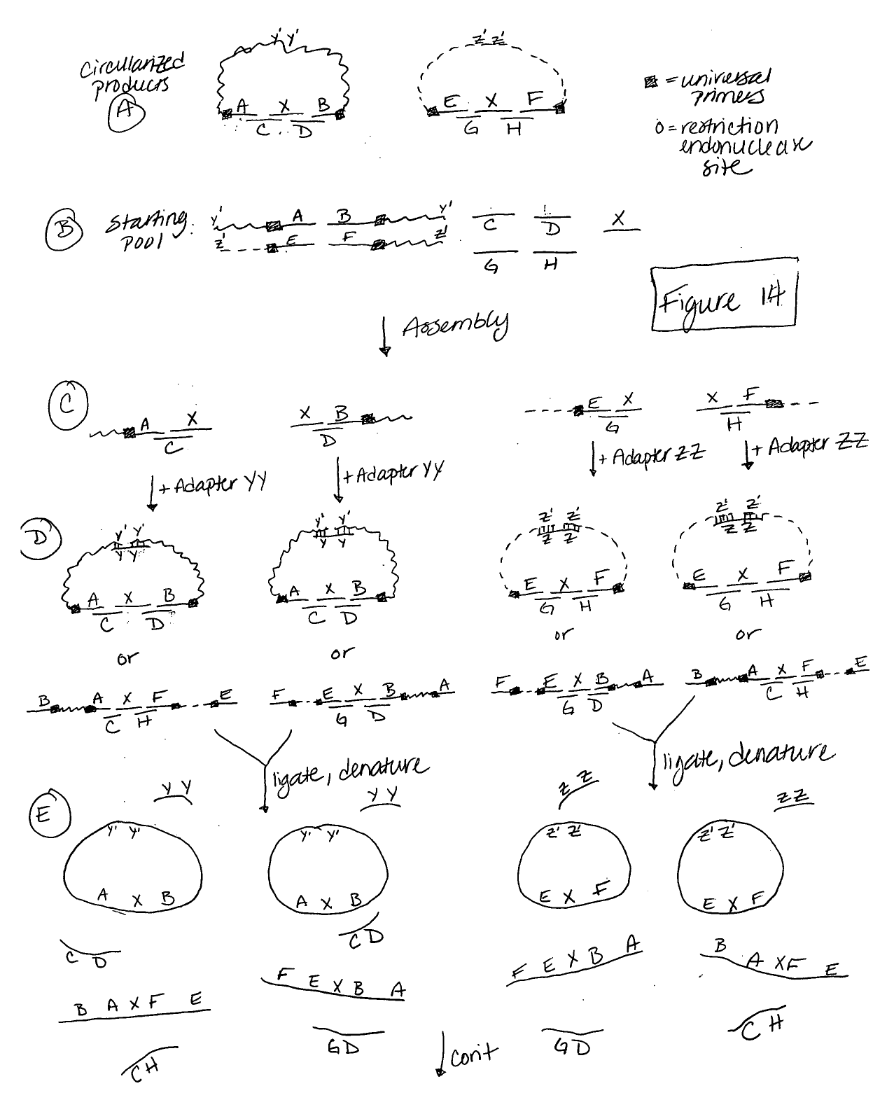

When conducting polymerase assembly multiplexing (PAM), homologous oligonucleotides can potentially act as crossover points leading to a mixture of full length products (FIGS. 11 and 12). Depending on the application, this can be a useful source of diversity, or a complication necessitating an additional separation step to obtain only the desired products. We have now discovered two strategies for accomplishing the selective separation of desired sequences from a mixture of crossover products: (1) selection by intermediate circularization and (2) selection by size. Both apply to PAM of polynucleotide constructs with one or more internal homologous regions.

In PAM (Tian et al., Nature 432: 1050-1054 (2004)), the order in which the oligonucleotide starting materials assemble to form polynucleotide constructs is defined by the mutual 5′ and 3′ complementarities of the oligos (Mullis et al., Cold Spring Harb. Symp. Quant. Biol. 51 pt 1: 263-273). The ends of each oligo can anneal to exactly one other oligo (except for the oligos at the end of a finished gene, which have a free end). This specificity of annealing ensures that only the desired full-length gene sequences will be assembled.

If there are sufficiently long regions of high homology among the genes to be synthesized in multiplexed format, however, this specificity can be lost. For example, when trying to synthesize two or more polynucleotide constructs that contain a highly homologous (or even identical) region X in a single pool, the common homologous region could lead to various assembled products in addition to the polynucleotide constructs of interest (see FIG. 11). This situation may arise when the homologous region X is at least as long as the construction oligonucleotide. This may occur, for example, when synthesizing polynucleotide constructs that encode closely related protein variants or proteins that share common domains. For example, as shown in FIGS. 11, A, B, C, D, E, F, G, H and X denote non-homologous construction oligos. By design, the 5′ end of X can hybridize with both C and G, and the 3′ end of X can hybridize with both D and H. This does not present a complication if the two sets of oligos do not come into contact with each other (e.g., they are in separate pools). However, if synthesis is performed in a single well, four distinct full-length products will be formed (identified by top strand only): AXB, AXF, EXB, and EXF (see FIG. 11D). Therefore, when dealing with a homologous region, the number of different products that may be formed is s^{x+1}, where s is the number of homologous sequences and x is the number of internal crossover points.

Internal homologous regions (e.g., two regions contained in the same sequence which are highly homologous or identical) are a special case because they have the potential to lead to polymerization in PAM. As shown in FIG. 12, assembly of the AXBXC nucleic acid (represented by the top strand only) could lead to a family of products represented by AX(BX)ₙC, where n is any nonnegative integer. The number of products generated by this assembly is theoretically infinite.

In certain embodiments, it may be desirable to allow this type of combinatorial complexity to occur. For example, this crossover feature of PAM can be exploited to quickly and cheaply generate large combinatorial libraries for applications such as domain shuffling for protein design, creation of a library of RNAi molecules, etc, creation of a library of aptamers, creation of library of Fab polypeptides, etc.

In other embodiments, it is desirable to minimize or eliminate combinatorial complexity and synthesize only a defined set of homologous sequences. This may be achieved by separately synthesizing genes containing homologous regions (to prevent crossover), for example, using separate pools that are mixed together in an ordered fashion to prevent crossover products. Alternatively, a variety of genes with homologous regions may be synthesized in a single pool and the undesired products may be removed using the separation techniques described below.

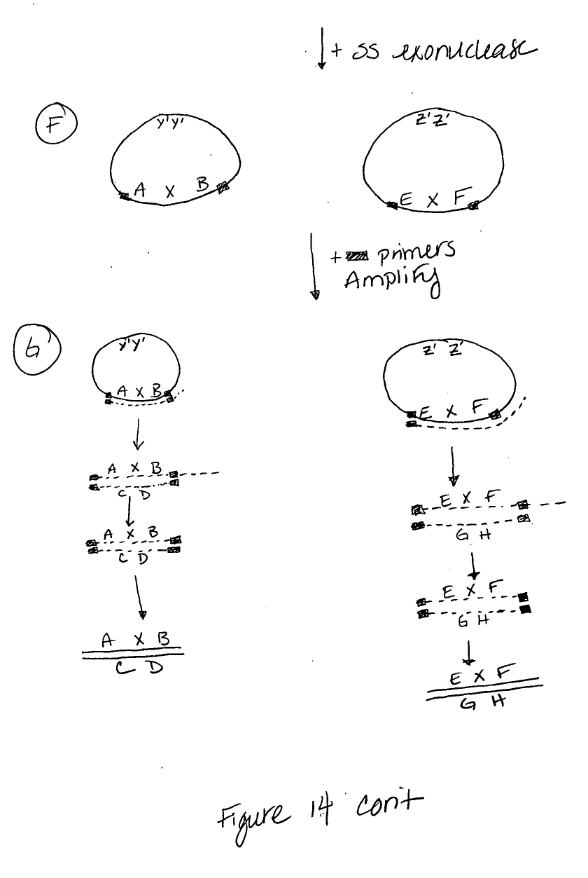

In one embodiment, undesired crossover products may be removed from a mixture of synthetic genes using a circle selection method. One embodiment of the circle selection method is illustrated in FIG. 13. The circle selection method takes advantage of the fact that circular single stranded DNA or double stranded DNA is exonuclease resistant. FIG. 13A illustrates two polynucleotide constructs that are desired to be constructed in a single pool (represented as a single strand for purposes of illustration). As shown in FIG. 13B, the terminal construction oligonucleotides are designed to form single stranded overhangs (which may optionally be formed by designing the construction oligos to contain an appropriate linker sequence) that allow the correct polynucleotide construct products to circularize, e.g., the complementary A/C oligos form a single stranded overhang that is complementary to a single stranded overhang formed by the complementary oligos B/D (represented by wavy lines) but are not complementary to a single stranded overhang formed by the F/H oligo pair (represented by dotted lines), etc. Therefore, only the correct products may circularize, while the incorrect crossover products (e.g., B-AXF-E and F-EXB-A) remain linear and may be degraded by an exonuclease leaving the circles intact (FIGS. 13D-F). The flanking regions and circularizing segment are assembled, and then the homologous linker X is added to the mixture. The desired sequences then form circles (FIGS. 13D and 13E), while the crossover products form linear sequences (FIG. 13F). These crossover products can be selectively degraded using an exonuclease. Then, an appropriate enzyme (e.g., a restriction enzyme or uracil DNA glycosylase (UDG)) can be added to linearize the circles and/or remove the circularizing segment (linkers), leaving only the desired products, e.g., AXB and EXF (represented by top strand only). As shown in FIGS. 13D and 13E, the circularized products may be partially double stranded (FIG. 13D) or alternatively may be completely double stranded (FIG. 13E). It is also possible to convert partially double stranded circles to fully double stranded circles using a polymerase and dNTPs.

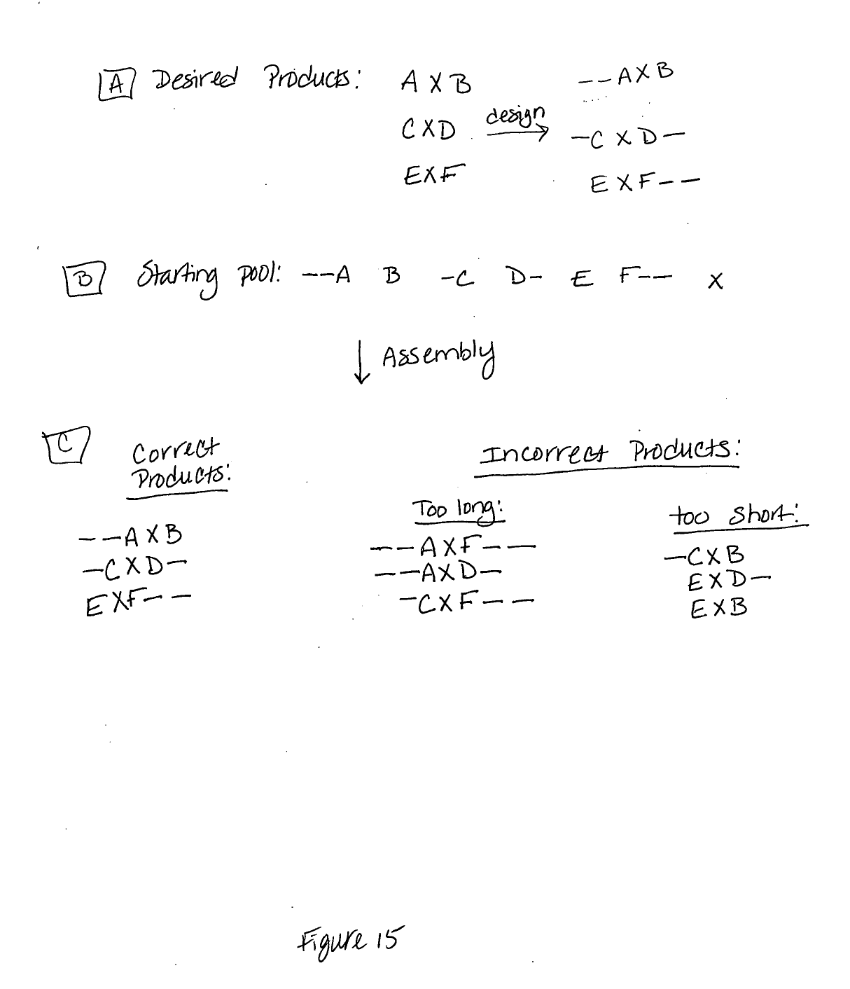

Another embodiment of the circle selection method is illustrated in FIG. 14. FIG. 14A shows the product polynucleotides that are desired to be synthesized in a single pool. FIG. 14B shows the construction oligonucleotides that define the polynucleotide constructs. The 5′ and 3′ most terminal construction oligonucleotides on the same strand contain flanking sequences that permit circularization of polynucleotide constructs that have been assembled in the proper order (e.g., oligos A and B, represented by wavy lines, and E and F, represented by dotted lines). After exposing the pool of polynucleotide constructs to hybridization conditions, liner sequences are added that are complementary to the flanking sequences of the terminal construction oligonucleotides. For example, as shown in FIGS. 14C and 14D, the adapter YY permits circularization of the AXB construct (e.g., by binding to the complementary Y′ regions) while the ZZ adapter permits circularization of the EXF construct (e.g., by binding to the complementary Z′ regions). However, incorrect crossover products (e.g., B-AXF-E and F-EXB-A) would have a combination of Y′ and Z′ complementary regions and therefore would not circularize upon exposure to the YY or ZZ adaptor oligonucleotides. The assembled constructs may then be ligated to form a covalently closed, partially single stranded circles and incorrect linear cross-over products (FIG. 14E). The constructs may then be denatured and subjected to a process to separate circles from linear nucleic acid strands (FIGS. 14E-14F). This may be accomplished, for example, using a size separation method (e.g., circles will migrate through a PAGE gel faster than linear products) or using a single stranded exonuclease to digest the linear strands while leaving the circles intact. The correct assembly products may then be produced by amplifying the appropriate region of the circular product using primers that bind to a region flanking the AXB and EXF products (FIG. 14G). It should be understood that the adapter oligos are represented by YY and ZZ merely for purposes of illustration. The adapter oligos may be any combination of sequences that is complementary to the appropriate pair of construction oligonucleotides (e.g., the sequence complementary to a region of the 5′ construction oligonucleotide need not be the same as the sequence complementary to a region of the 3′ construction oligonucleotide).

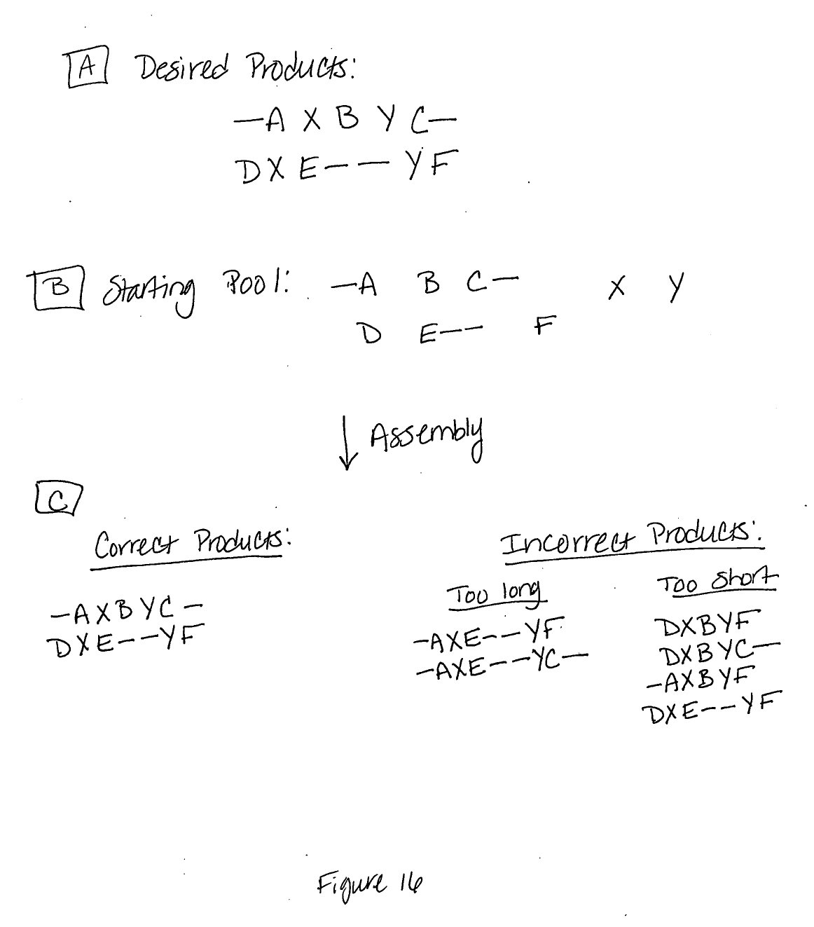

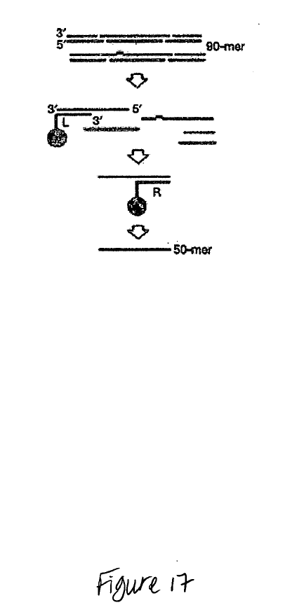

In another embodiment, undesired crossover products may be removed from a mixture of synthetic polynucleotide constructs using the size selection method which is illustrated in FIGS. 15 and 16. The size selection method takes advantage of the fact that the mobility of double stranded DNA is a function of its size, and thus DNA of different lengths can be separated, for example, via gel or column chromatography. In this embodiment, the initial polynucleotide constructs are designed such that the desired products have different lengths than all of the crossover products (see e.g., FIGS. 15A and 16A). For example, in one embodiment, the oligonucleotides are designed such that all of the desired products are about the same size, and any crossover products have significantly different sizes. This may be accomplished by designing the construction oligonucleotides such that the crossover point is in a different position in each of the target sequences. For example, as illustrated in FIG. 15, if the desired sequences are AXB, CXD, and EXF, and the A, B, C, C, E, F, and X are all approximately the same length, the sequences can be “padded” (e.g., the addition of extra bases or series of bases, represented as dashes) (FIG. 15B) to yield desired products having the same length, e.g., --AXB, -CXD-, and EXF--, and undesired crossover products having different lengths, e.g., --AXF--, --AXD-, -CXF--, -CXB, EXED-, or EXB (FIG. 15C). The genes can be assembled in multiplexed format and the desired products separated from the crossover products by size selection. The padding units can then be removed using a restriction enzyme or UDG. In certain embodiments, such size selection techniques may be achieved merely through careful design of the construction oligonucleotides without the need to pad the oligos, e.g., the A, B, C, C, E, F, and X are naturally different sizes and will permit the distinction between correct vs. incorrect products.

The degree of difference in length needed to distinguish the products may be determined based on the separation method to be used. For example, if the size separation will be performed by gel electrophoresis, then a separation resolution and size differential of about +/−5-10% of the full gene sequence may be reasonable.

In another embodiment, if an internal region of DNA with known markers can be selectively excised, a single size selection could be used on sequences with more than one region of homology. This embodiment is illustrated in FIG. 16 for products AXBYC and DXEYF which may be synthesized in a single pool, for example, as -AXBYC- and DXE--YF (FIG. 16A) using the construction oligonucleotides shown in FIG. 16B. Of the 8 possible products (FIG. 16C), the 2 desired products each contain 2 units of padding (“-”), while the 6 crossover products at X or Y contain either 0, 1, 3, or 4 units of padding (FIG. 16C). The regions of internal padding may then be excised, for example, using a restriction endonuclease (e.g. a type IIS restriction endonuclease). The fragments may then be exposed to hybridization and ligation conditions to form the correct, unpadded construct.

In another embodiment, when multiple internal homologous regions are present, separate assembly and separation steps may be performed for each homologous region. The resulting gene fragments will then be unique and can be assembled via PAM. This is a “linear” strategy which scales in complexity as the number of homologous regions. As the molecule length grows, conventional methods of error-reduction become prohibitively cumbersome and costly. Set forth below are tools for dramatically reducing errors in large-scale gene synthesis.

In other embodiments, multiplex synthesis of sequences containing homologous regions may be achieved by careful design of the construction oligonucleotides. For example, the construction oligonucleotides may be codon remapped to reduce the level of homology while still maintaining or minimally changing any polypeptide sequence encoded by the nucleic acid. Additionally, the areas of complementarity between two or more construction oligonucleotides may be carefully chosen to reduce the level of homology in undesired regions of hybridization (see e.g., PCT Publication WO 00/43942). Methods for oligonucleotide design and codon remapping may be facilitated through the aid of computer design using, for example, DNAWorks (supra), Gene2Oligo (supra), or the implementation methods and systems discussed further below.

7. Error Reduction

When using pairs of complementary DNA strands for error recognition, each strand in the pair may contain errors at some frequency, but when the strands are annealed together, the chance of errors occurring at a correlated location on both strands is very small, with an even smaller chance that such a correlation will produce a correctly matched Watson-Crick base pair (e.g. A-T, G-C). For example, in a pool of 50-mer oligonucleotides, with a per-base error rate of 1%, roughly 60% of the pool (0.9950) will have the correct sequence, and the remaining forty percent will have one or more errors (primarily one error per oligonucleotide) in random positions. The same would be true for a pool composed of the complementary 50-mer. After annealing the two pools, approximately 36% (0.62) of the DNA duplexes will have correct sequence on both strands, 48% (2×0.4×0.6) will have an error on one strand, and 16% (0.42) will have errors in both strands. Of this latter category, the chance of the errors being in the same location is only 2% (1/50) and the chance of these errors forming a Watson-Crick base pair is even less (1/3×1/50). These correlated mismatches, which would go undetected, then comprise 0.11% of the total pool of DNA duplexes (16×1/3×1/50). Removal of all detectable mismatch-containing sequences would thus enrich the pool for error-free sequences (i.e. reduce the proportion of error-containing sequences) by a factor of roughly 200 (0.6/0.4 originally for the single strands vs. 0.36/0.0011 after mismatch detection and removal). Furthermore, the remaining oligonucleotides can then be dissociated and re-annealed, allowing the error-containing strands to partner with different complementary strands in the pool, producing different mismatch duplexes. These can also be detected and removed as above, allowing for further enrichment for the error-free duplexes. Multiple cycles of this process can in principle reduce errors to undetectable levels. Since each cycle of error control may also remove some of the error-free sequences (while still proportionately enriching the pool for error-free sequences), alternating cycles of error control and DNA amplification can be employed to maintain a large pool of molecules.

In one embodiment, the number of errors detected and corrected may be increased by melting and reannealing a pool of DNA duplexes prior to error reduction. For example, if the DNA duplexes in question have been amplified by a technique such as the polymerase chain reaction (PCR) the synthesis of new (perfectly) complementary strands would mean that these errors are not immediately detectable as DNA mismatches. However, melting these duplexes and allowing the strands to re-associate with new (and random) complementary partners would generate duplexes in which most errors would be apparent as mismatches (FIG. 8).

Many of the methods for error reduction can be used together at multiple points during the assembly process. For example, error reduction may be applied to the construction oligonucleotides, subassemblies, and/or the final polynucleotide constructs. In an exemplary embodiment, error filtration by means of selective hybridization may be applied to the construction oligonucleotides, one or more error filtration, error neutralization, and/or error correction process may be applied to subassemblies/polynucleotide constructs ranging in size from about 500 to about 10,000 bases, and error correction process may be applied to subassemblies/polynucleotide constructs of about 10,000 bases or more.

In one aspect, the invention provides methods for increasing the fidelity of a polynucleotide pool by removing polynucleotide copies that contain errors via hybridization to one or more selection oligonucleotides. This type of error filtration process may be carried out on oligonucleotides at any stage of assembly, for example, construction oligonucleotides, subassemblies, and in some cases larger polynucleotide constructs. Additionally, error filtration using selection oligonucleotides may be conducted before and/or after amplification of the polynucleotide pool. In an exemplary embodiment, error filtration using selective oligonucleotides is used to increase the fidelity of the pool of construction oligonucleotides before and/or after amplification. An illustrative embodiment of error filtration through hybridization to selection oligonucleotides is shown in FIG. 17. A pool of construction oligonucleotides has been amplified using universal primers. Some of the construction oligonucleotides contain errors which are represented by a bulge in the strand. These errors may have arisen from the initial synthesis of the construction oligonucleotides or may have been introduced during the amplification process. The pool of construction oligonucleotides is then denatured to produce single strands and contacted with at least one pool of selection oligonucleotides under hybridization conditions. The pool of selection oligonucleotides comprises one or more selection oligonucleotides complementary to each of the construction oligonucleotides in the pool (e.g., the pool of selection oligos is at least as large as the pool of construction oligonucleotides, and in some cases may comprise, e.g., twice as many different oligonucleotides as compared to the pool of construction oligonucleotides). Copies of construction oligonucleotides that do not perfectly pair with a selection oligonucleotide (e.g., there is a mismatch) will not hybridize as tightly as perfectly matched copies and can be removed from the pool by controlling the stringency of the hybridization conditions. After removal of the oligonucleotides containing mismatches, the perfectly matched copies of the construction oligonucleotides may be removed by increasing the stringency conditions to elute them off of the selection oligonucleotides. In an exemplary embodiment, the selection oligonucleotides may be end immobilized (e.g., via chemical linkage, biotin/streptavidin, etc.) to facilitate removal of oligonucleotide copies containing errors. For example, the selection oligonucleotides may be immobilized on beads before or after hybridization to the pool of construction oligonucleotides. The beads may then be pelleted, or loaded onto a colurn, and exposed to different stringency conditions to remove copies of construction oligonucleotides containing a mismatch with the selection oligonucleotide. In certain embodiments, it may be desirable to submit the oligonucleotides to iterative rounds of amplification and error filtration through hybridization to a pool of selection oligonucleotides thereby increasing the number of copies of oligonucleotides in the pool while maintaining, or preferably increasing, the fidelity of the pool (e.g., increasing the number of error free copies in the pool).

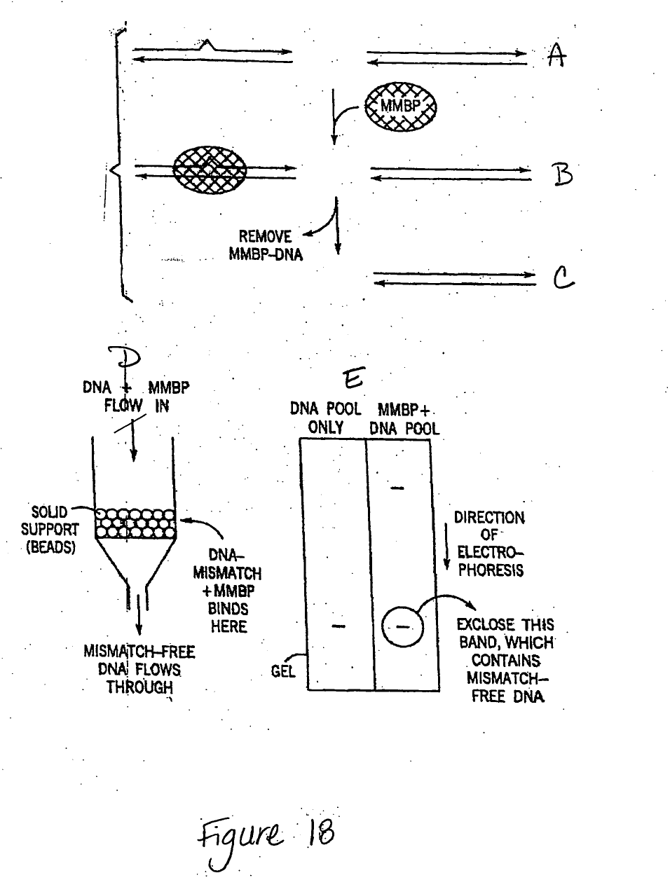

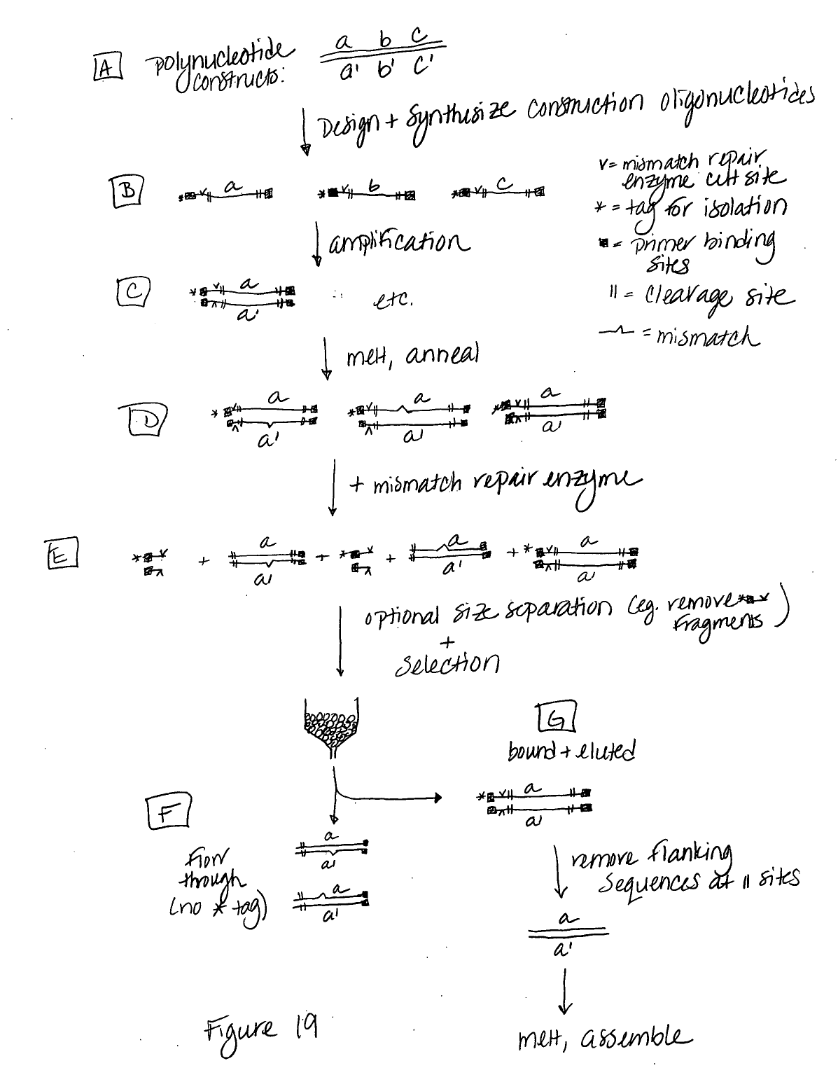

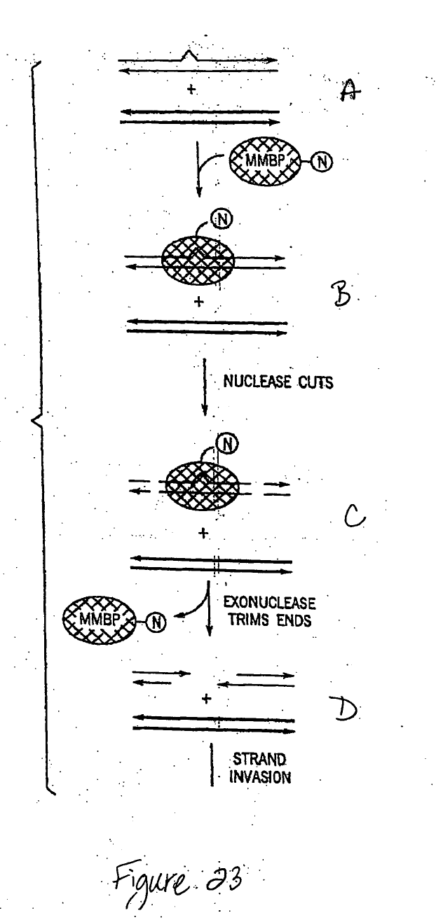

It should be noted that in some instances, the mismatch between the construction and selection oligonucleotides will arise from a sequence error in the selection oligonucleotide thereby removing an error free construction oligonucleotide from the pool. However, the net effect will still be increased fidelity of the construction oligonucleotide pool. In one embodiment, the fidelity of the selection oligonucleotide pool may be increased simultaneously with an increase in the fidelity of the construction oligonucleotide pool. For example, after mixing the pools of construction and selection oligonucleotide pools under hybridization conditions, the mixture may be exposed to one more agents that cleave a mismatched polynucleotide or crosslink to a mismatched polynucleotide (see e.g., FIGS. 18, 20-22 and 24 discussed below). This process will effectively remove copies of both the selection and construction oligonucleotides in the mixture that contained a mismatch when hybridized together. In subsequent rounds of filtration using the same selection oligonucleotide pool, the fidelity of this pool will be increased thereby reducing the number of error free construction oligonucleotides removed from the pool due to an error in a selection oligonucleotide. Additionally, use of an agent that cleaves or crosslinks to a mismatched polynucleotide may be used to facilitate removal of the mismatched copy from the pool of oligonucleotides (see e.g., FIGS. 21-26 discussed below).

FIG. 18 illustrates an exemplary method for removing sequence errors using mismatch binding agent. An error in a single strand of DNA causes a mismatch in a DNA duplex. A mismatch binding protein (MMBP), such as a dimer of MutS, binds to this site on the DNA. As shown in FIG. 18A, a pool of DNA duplexes contains some duplexes with mismatches (left) and some which are error-free (right). The 3′-terminus of each DNA strand is indicated by an arrowhead. An error giving rise to a mismatch is shown as a raised triangular bump on the top left strand. As shown in FIG. 18B, a MMBP may be added which binds selectively to the site of the mismatch. The MMBP-bound DNA duplex may then be removed, leaving behind a pool which is dramatically enriched for error-free duplexes (FIG. 18C). In one embodiment, the DNA-bound protein provides a means to separate the error-containing DNA from the error-free copies (FIG. 18D). The protein-DNA complexes can be captured by affinity of the protein for a solid support functionalized, for example, with a specific antibody, immobilized nickel ions (protein is produced as a his-tag fusion), streptavidin (protein has been modified by the covalent addition of biotin) or other such mechanisms as are common to the art of protein purification. Alternatively, the protein-DNA complex is separated from the pool of error-free DNA sequences by a difference in mobility, for example, using a size-exclusion column chromatography or by electrophoresis (FIG. 18E). In this example, the electrophoretic mobility in a gel is altered upon MMBP binding: in the absence of MMBP all duplexes migrate together, but in the presence of MMBP, mismatch duplexes are retarded (upper band). The mismatch-free band (lower) is then excised and extracted.

In an exemplary embodiment, the methods described herein utilize error filtration that involves contacting a pool of nucleic acid duplexes with a mutS polypeptide in the presence of ATP (see e.g., Junop et al., Mol. Cell 7: 1-12 (2001); Schofield et al., J. Biol. Chem. 276: 28291-28299 (2001); and Lamers et al., J. Biol. Chem. 279: 43879-43885 (2004)). The ATP increases the affinity of the mutS for the mismatched DNA strand thereby facilitating removal of the mismatch duplexes from the mixture. For example, ATP may be added to the reaction in about a 1-100 fold, 1-10 fold, or 2-5 molar excess as compared to mutS. In an exemplary embodiment, the amount of ATP included in the reaction is sufficient to increase the affinity and/or selectivity of a mutS protein for a duplex comprising a mismatch. The ATP may increase the affinity of the mutS protein for a duplex comprising a mismatch to the low nanomolar range, e.g., to less than about 50 nm, 20 nm, 10 nm, 5 nm, 1 nm, or less. In one embodiment, the amount of mutS needed to perform an error correction process may be significantly reduced by the addition of ATP to the reaction. For example, in the presence of ATP, the amount of mutS needed to conduct an error correction process may be reduced by at least 2-fold, 5-fold, 10-fold, 100-fold, or more. The mismatch duplexes may be removed from the pool of oligonucleotides using the methods described above (e.g., gel electrophoresis, size exclusion chromatography, affinity chromatography, etc.).

In another exemplary embodiment, a DNA glycosylase may be used as a mismatch binding agent in an error filtration process. Exemplary DNA glycosylases include, for example, thymine DNA glycosylase which recognizes T/G mismatches (e.g., GenBank Accession No. AF117602), a mutant thymine DNA glycosylase which recognizes a mismatch but has reduced catalytic activity (see e.g., U.S. Patent Publication No. 2004/0014083 and Hsu et al., Carcinogenesis 15: 1657-62 (1994)), mutY which recognizes G/A mismatches (e.g., GenBank Accession Nos. AF121797 (Streptomyces), U63329 (Human), AA409965 (Mus musculus) and AF056199 (Streptomyces)), and a mutant mutY which recognizes a mismatch (including A/G and A/C mismatches) but has reduced catalytic activity (see e.g., U.S. Patent Publication No. 2004/0014083, and Michaels et al., Proc. Natl. Acad. Sci. U.S.A., 89(15):7022-5 (1992)). In one embodiment, the mismatch binding agent is a mutant E. coli mutY polypeptide having E37S, V45N, G116D, D138N or K142A mutations (Lu et al., J. Biol. Chem., 271(39):24138-43 (1996); Guan et al., Nat. Struct. Biol., 5(12):1058-64 (1998); and Wright et al., J. Biol. Chem., 274(41):29011-18 (1999)).

In another embodiment, a mismatch binding agent is a mutS polypeptide which recognizes any mismatched base and small (1-5 bases) single stranded loops. Exemplary mutS polypeptides include, for example, polypeptides encoded by nucleic acids with the following GenBank accession Nos.: AF146227 (Mus musculus), AF193018 (Arabidopsis thaliana), AF144608 (Vibrio parahaemolyticus), AF034759 (Homo sapiens), AF104243 (Homo sapiens), AF007553 (Thermus aquaticus caldophilus), AF109905 (Mus musculus), AF070079 (Homo sapiens), AF070071 (Homo sapiens), AH006902 (Homo sapiens), AF048991 (Homo sapiens), AF048986 (Homo sapiens), U33117 (Thermus aquaticus), U16152 (Yersinia enterocolitica), AF000945 (Vibrio cholarae), U698873 (Escherichia coli), AF003252 (Haemophilus influenzae strain b (Eagan), AF003005 (Arabidopsis thaliana), AF002706 (Arabidopsis thaliana), L10319 (Mouse), D63810 (Thermus thermophilus), U27343 (Bacillus subtilis), U71155 (Thermotoga maritima), U71154 (Aquifex pyrophilus), U16303 (Salmoraella typhimurium), U21011 (Mus musculus), M84170 (S. cerevisiae), M84169 (S. cerevisiae), M18965 (S. typhimurium) and M63007 (Azotobacter vinelandii). The mismatch binding agent may also be a mutant mutS protein that recognizes mismatches but has reduced catalytic activity (see, e.g., U.S. Patent Publication No. 2004/0014083 and Wu et al., J. Biol. Chem., 274(9):5948-52 (1999)). In another embodiment, the mismatch binding agent may be a MSH2 protein, e.g., a eukaryotic homolog of mutS. Exemplary MSH2 proteins include, for example, polypeptides encoded by the nucleic acids having GenBank accession Nos.: AF109243 (Arabidopsis thaliana), AF030634 (Neurospora crassa), AF002706 (Arabidopsis thaliana), AF026549 (Arabidopsis thaliana), L47582 (Homo sapiens), L47583 (Homo sapiens), L47581 (Homo sapiens) and M84170 (S. cerevisiae). The mismatch binding agent may also be a mutant MSH2 protein that recognizes mismatches but has reduced catalytic activity (see e.g., U.S. Patent Publication No. 2004/0014083) such as a S. cerevisiae mutant MSH2 having a G693D or a G855D mutation (Alani et al., Mol. Cell. Biol., 17(5):2436-47 (1997)), or a human mutant MSH2 having a fragment encoding 195 amino acids within the C-terminal domain of hMSH-2 or having a K675R mutation (Whitehouse et al., Biochem. Biophys. Res. Commun., 232(1):10-3 (1997); and laccarino et al., EMBO J., 17(9):2677-86 (1998)).

In another embodiment, a mismatch binding agent may comprise a mixture of two or more mismatching binding agents. For example, a mixture of two or more mismatching binding agents that have different specificity or affinity for a different base pair mismatches, insertions, or deletions may be used so as to provide efficient recognition of any potential base error.

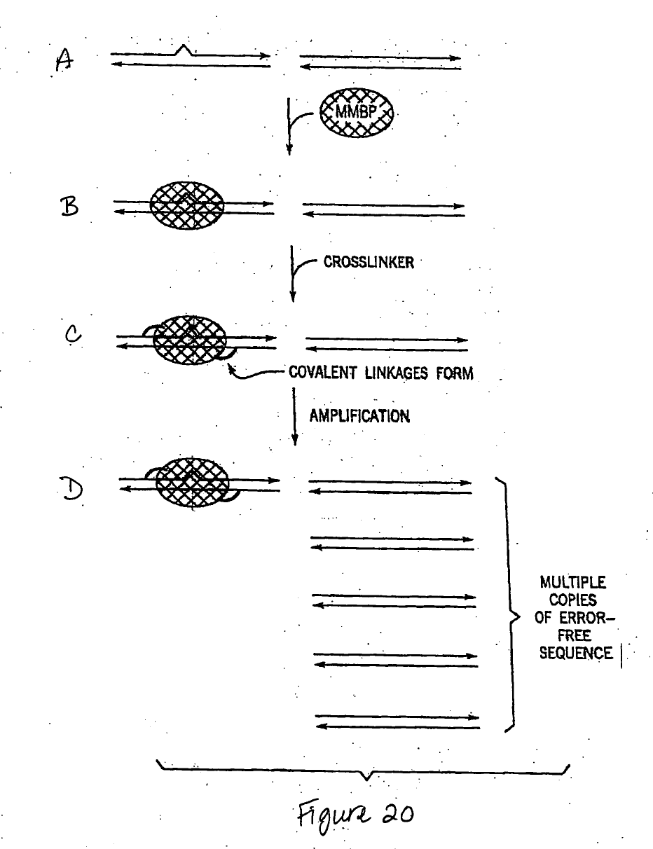

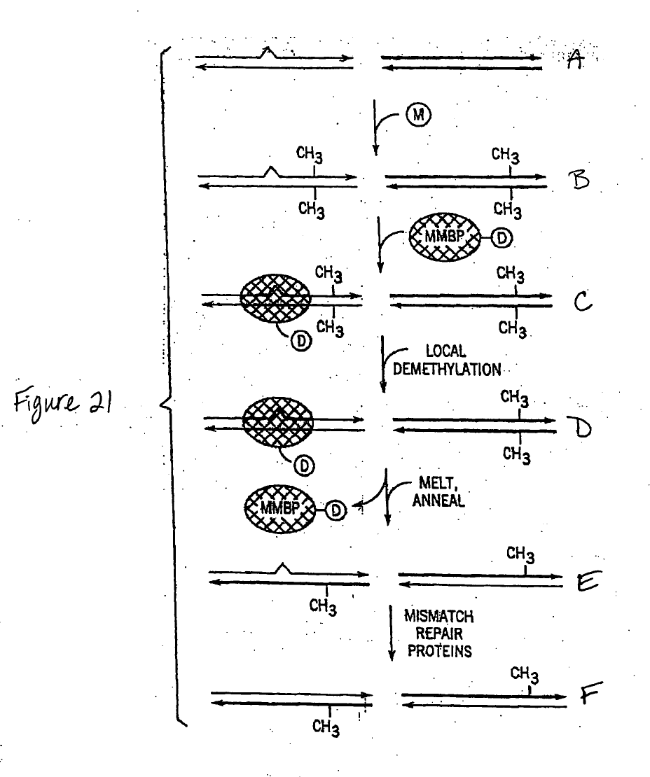

FIG. 19 illustrates another method for removing sequence errors using a mismatch binding agent. This method of error filtration may be used to remove errors from construction oligonucleotides, subassemblies, and/or polynucleotide constructs. FIG. 19A shows the polynucleotide constructs to be prepared using the methods described herein. Overlapping construction oligonucleotides defining the polynucleotide constructs are designed and synthesized. The construction oligonucleotides comprise universal tags that comprise a universal primer binding site, a mismatch repair enzyme cut site, a tag for isolation of the oligonucleotide, and a restriction endonuclease cleavage site at the junction between the universal tags and the construction oligonucleotide (FIG. 19B). In various embodiments, the universal tags at one or both of the 5′ and 3′ flanking sequences may comprise a mismatch repair enzyme cut site. The construction oligonucleotides are then amplified (FIG. 19C) followed by an optional round of denaturation and renaturation to form a pool of double stranded construction oligonucleotides wherein some copies contain a mismatch, insertion, or deletion (FIG. 19D). The pool of construction oligonucleotides is then contacted with a mismatch repair enzyme that cuts at the mismatch repair enzyme cut site located in one or more of the universal tags (FIG. 19E). This cleavage removes the tag for isolation from the construction oligonucleotide molecule thereby producing a pool of construction oligonucleotides wherein duplexes containing mismatches no longer contain the tag for isolation and error free duplexes still contain the tag for isolation. The short fragments containing the cleaved universal tags may optionally be removed prior to separation or may be removed at a later stage (e.g., by size separation using column chromatography, gel electrophoresis, etc.). The pool of construction oligonucleotides is then subjected to a separation process such passage through a column functionalized with a binding partner for the isolation tag (e.g., use of a streptavadin column for isolation of biotin tags). The mismatch containing sequences that have been cleaved by the mismatch repair enzyme do not contain the isolation tag and will not bind to the column (e.g., they will flow through the column) (FIG. 19F). The error free sequences that were not cleaved by the mismatch repair enzyme will bind to the column and may be eluted, optionally, after washing to remove any copies of the cleaved construction oligonucleotides that bound to the column non-specifically (FIG. 19G). The eluted construction oligonucleotides may then optionally be subjected to another round of error filtration and/or amplification. The pool of purified construction oligonucleotides may then be cleaved (e.g., using a type IIS restriction endonuclease) to remove the universal tags and assembled into subassemblies and/or polynucleotide constructs using the methods described herein. In an exemplary embodiment, the method illustrated in FIG. 19 utilizes a mutHLS complex as the mismatch repair enzyme. The mutHLS complex carries out double stranded cleavage at d(GATC) sites (see e.g., Smith and Modrich, Proc. Natl. Acad. Sci. USA 94: 6847-6850 (1997)).

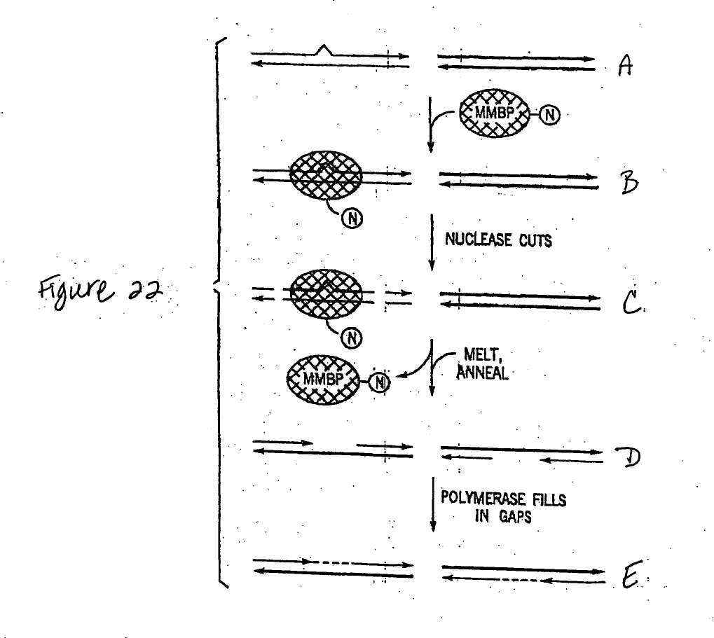

FIG. 20 illustrates an exemplary method for neutralizing sequence errors using a mismatch binding agent. In this embodiment, the error-containing DNA sequence is not removed from the pool of DNA products. Rather, it becomes irreversibly complexed with a mismatch recognition protein by the action of a chemical crosslinking agent (for example, dimethyl suberimidate, DMS), or of another protein (such as MutL). The pool of DNA sequences is then amplified (such as by the polymerase chain reaction, PCR), but those containing errors are blocked from amplification, and quickly become outnumbered by the increasing error-free sequences. FIG. 20A illustrates an exemplary pool of DNA duplexes containing some duplexes with mismatches (left) and some which are error-free (right). A MMBP may be used to bind selectively to the DNA duplexes containing mismatches (FIG. 20B). The MMBP may be irreversibly attached at the site of the mismatch upon application of a crosslinking agent (FIG. 20C). In the presence of the covalently linked MMBP, amplification of the pool of DNA duplexes produces more copies of the error-free duplexes (FIG. 20D). The MMBP-mismatch DNA complex is unable to participate in amplification because the bound protein prevents the two strands of the duplex from dissociating. For long DNA duplexes, the regions outside the MMBP-bound site may be able to partially dissociate and participate in partial amplification of those (error-free) regions.

As increasingly longer sequences of DNA are generated, the fraction of sequences which are completely error-free diminishes. At some length, it becomes likely that there will be no molecule in the entire pool which contains a completely correct sequence. Thus, for the generation of extremely long segments of DNA, it can be useful to produce smaller units first which can be subjected to the above error control approaches. Then these segments can be combined to yield the larger full length product. However, if errors in these extremely long sequences can be corrected locally, without removing or neutralizing the entire long DNA duplex, then the more complex stepwise assembly process can be avoided.

Many biological DNA repair mechanisms rely on recognizing the site of a mutation (error) and then using a template strand (most likely error-free) to replace the incorrect sequence. In the de novo production of DNA sequences, this process is complicated by the difficulty of determining which strand contains the error and which should be used as the template. One solution to this problem relies on using the pool of other sequences in the mixture to provide the template for correction. These methods can be very robust: even if every strand of DNA contains one or more errors, as long as the majority of strands have the correct sequence at each position (expected because the positions of errors are generally not correlated between strands), there is a high likelihood that a given error will be replaced with the correct sequence. FIGS. 21-22 and 24-27 present exemplary procedures for performing this sort of local error correction.

FIG. 21 illustrates an exemplary method for carrying out strand-specific error correction. In replicating organisms, enzyme-mediated DNA methylation is often used to identify the template (parent) DNA strand. The newly synthesized (daughter) strand is at first unmethylated. When a mismatch is detected, the hemimethylated state of the duplex DNA is used to direct the mismatch repair system to make a correction to the daughter strand only. However, in the de novo synthesis of a pair of complementary DNA strands, both strands are unmethylated, and the repair system has no intrinsic basis for choosing which strand to correct. Methylation and site-specific demethylation are employed to produce DNA strands that are selectively hemi-methylated. A methylase, such as the Dam methylase of E. coli, is used to uniformly methylate all potential target sites on each strand. The DNA strands are then dissociated, and allowed to re-anneal with new partner strands. A new protein is applied, a fusion of a mismatch binding protein (MMBP) with a demethylase. This fusion protein binds only to the mismatch, and the proximity of the demethylase removes methyl groups from either strand, but only near the site of the mismatch. A subsequent cycle of dissociation and annealing allows the (demethylated) error-containing strand to associate with a (methylated) strand which is error-free in this region of its sequence. (This should be true for the majority of the strands, since the locations of errors on complementary strands are not correlated.) The hemi-methylated DNA duplex now contains all the information needed to direct the repair of the error, employing the components of a DNA mismatch repair system, such as that of E. coli, which employs MutS, MutL, MutH, and DNA polymerase proteins for this purpose. The process can be repeated multiple times to ensure all errors are corrected.

FIG. 21A shows two DNA duplexes that are identical except for a single base error in the top left strand, giving rise to a mismatch. The strands of the right hand duplex are shown with thicker lines. Methylase (M) may then be used to uniformly methylates all possible sites on each DNA strand (FIG. 21 B). The methylase is then removed, and a protein fusion is applied, containing both a mismatch binding protein (MMBP) and a demethylase (D) (FIG. 21C). The MMBP portion of the fusion protein binds to the site of the mismatch thus localizing the fusion protein to the site of the mismatch. The demethylase portion of the fusion protein may then act to specifically remove methyl groups from both strands in the vicinity of the mismatch (FIG. 21D). The MMBP-D protein fusion may then be removed, and the DNA duplexes may be allowed to dissociated and re-associate with new partner strands (FIG. 21E). The error-containing strand will most likely re-associate with a complementary strand which a) does not contain a complementary error at that site; and b) is methylated near the site of the mismatch. This new duplex now mimics the natural substrate for DNA mismatch repair systems. The components of a mismatch repair system (such as E. coli MutS, MutL, MutH, and DNA polymerase) may then be used to remove bases in the error-containing strand (including the error), and uses the opposing (error-free) strand as a template for synthesizing the replacement, leaving a corrected strand (FIG. 21F).

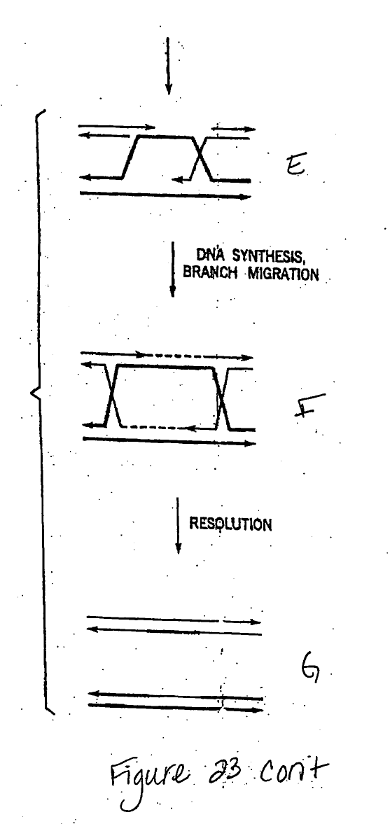

FIG. 22 illustrates an exemplary method for local removal of DNA on both strands at the site of a mismatch. Various proteins can be used to create a break in both DNA strands near an error. For example, an MMBP fusion to a non-specific nuclease (such as DNAseI) can direct the action of the nuclease (N) to the mismatch site, cleaving both strands. Once the break is generated, homologous recombination can be employed to use other strands (most of which will be error-free at this site) as template to replace the excised DNA. For example, the RecA protein can be used to facilitate single strand invasion, and early step in homologous recombination. Alternatively, a polymerase can be employed to allow broken strands to reassociate with new full-length partner strands, synthesizing new DNA to replace the error. For example, FIG. 22A shows two DNA duplexes that identical except that one contains a single base error as in FIG. 22A. In one embodiment, a protein, such as a fusion of a MMBP with a nuclease (N), may be added and will bind at the site of the mismatch (FIG. 22B). Alternatively, a nuclease with specificity for single-stranded DNA can be employed, using elevated temperatures to favor local melting of the DNA duplex at the site of the mismatch. (In the absence of a mismatch, a perfect DNA duplex will be less likely to melt.) An endonuclease, such as that of the MMBP-N fusion, may be used to make double-stranded breaks near the site of the mismatch (FIG. 22C). The MMBP-N complex is then removed, along with the bound short region of DNA duplex around the mismatch (FIG. 22D). Melting and re-annealing of partner strands produces some duplexes with single-stranded gaps. A DNA polymerase may then be used to fill in the gaps, producing DNA duplexes without the original error (FIG. 22E).

In an exemplary embodiment, the error correction process outlined in FIG. 22 may be carried out using a resolvase protein which introduces double stranded breaks in heteroduplex DNA at the sites of mismatches. Exemplary resolvase proteins include, for example, T7 endonuclease I and T4 endonuclease VII (see e.g., Young and Dong, Nucleic Acids Res. 32: e59 (2004); Qiu et al., Appl. Environ. Microbiol. 67: 880-887 (2001); Picksley et al., J. Mol. Biol. 212: 723-735 (1990); Mashal et al., Nature Genet. 9: 177-183 (1995); B. Kemper (1997) in DNA Damage and Repair, eds. J. Nickoloff and M. Hoekstra (Humana Press, Totosw, N.J.), 1, pp. 179-204). T7 endonuclease I may be purchased commercially, for example, from New England Biolabs (Beverly, Mass.) and t4 endonuclease VII may be purchased commercially, for example, from USB (Cleveland, Ohio).

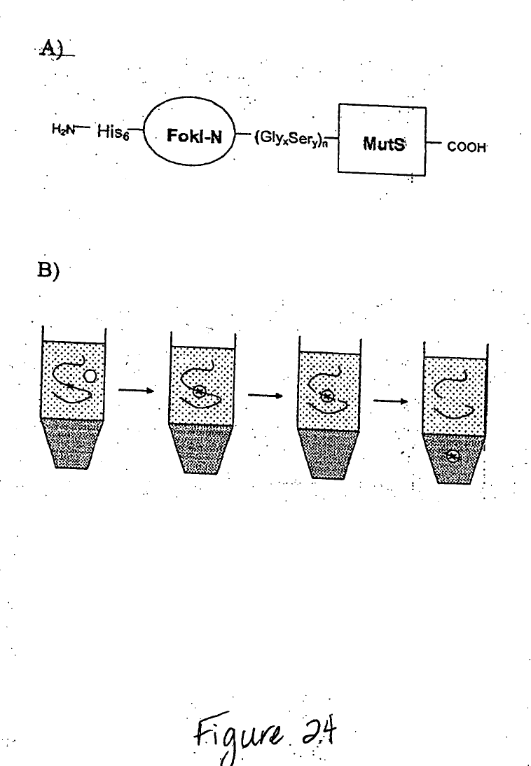

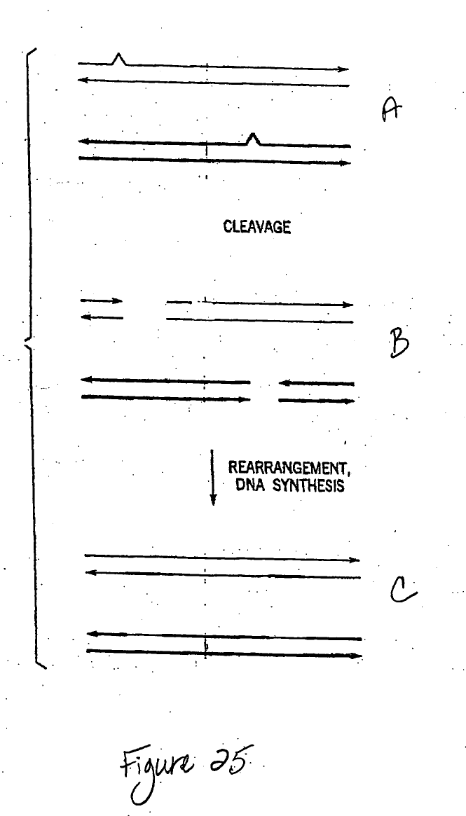

FIG. 23 illustrates a process similar to that of FIG. 22, however, in this embodiment, double-stranded gaps in DNA duplexes are repaired using the protein components of a recombination repair pathway. (Note that in this case no global melting and re-annealing of DNA strands is required, which can be preferable when dealing with especially large DNA molecules, such as genomic DNA.) For example, FIG. 23A shows two DNA duplexes (as in FIG. 22A), identical except that one contains a single base mismatch. As in FIG. 22B, a protein, such as a fusion of a MMBP with a nuclease (N), is added to bind at the site of the mismatch (FIG. 22B). As in FIG. 22C, an endonuclease, such as that of the MMBP-N fusion, may be used to make double-stranded breaks around the site of the mismatch (FIG. 23C). An exemplary MMBP-N fusion protein is illustrated in FIG. 24. Protein components of a DNA repair pathway, such as the RecBCD complex, may then be employed to further digest the exposed ends of the double-stranded break, leaving 3′ overlaps (FIG. 23D). Subsequently, protein components of a DNA repair pathway, such as the RecA protein, are employed to facilitate single strand invasion of the intact DNA duplex, forming a Holliday junction (FIG. 23E). A DNA polymerase may then be used to synthesize new DNA, filling in the single-stranded gaps (FIG. 23F). Finally, protein components of a DNA repair pathway may be employed, such as the RuvC protein, to resolve the Holliday junction (FIG. 23G). The two resulting DNA duplexes do not contain the original error. Note that there can be more than one way to resolve such junctions, depending on migration of the branch points.

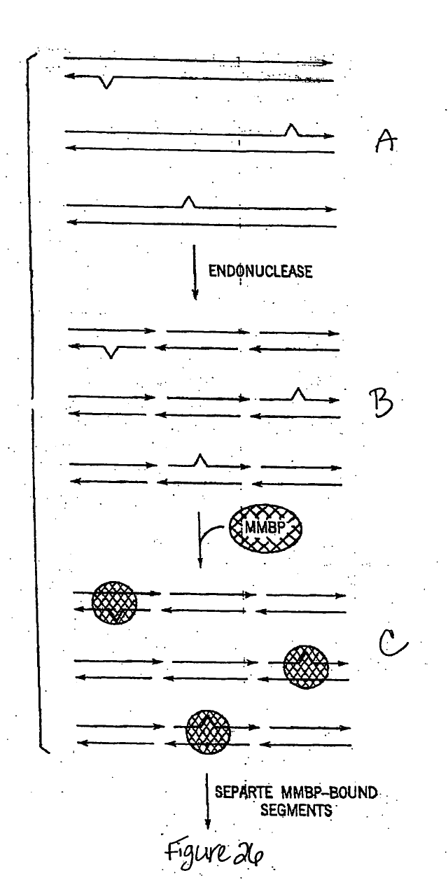

It is important to make clear that the methods described herein are capable of generating large error-free DNA sequences, even if none of the initial DNA products are error-free. FIG. 25 summarizes the effects of the methods of FIG. 22 (or equivalently, FIG. 23) applied to two DNA duplexes, each containing a single base (mismatch) error. For example, FIG. 25A illustrates two DNA duplexes, identical except for a single base mismatch in each, at different locations in the DNA sequence. Mismatch binding and localized nuclease activity are then used to generated double-stranded breaks which excise the errors (FIG. 25B). Recombination repair (as in FIG. 23) or melting and reassembly (as in FIG. 22) are employed to generate DNA duplexes where each excised error sequence has been replaced with newly synthesized sequence, each using the other DNA duplex as template (and unlikely to have an error in that same location) (FIG. 25C). Note that complete dissociation and re-annealing of the DNA duplexes is not necessary to generate the error-free products (if the methods shown in FIG. 23 are employed).

A simple way to reduce errors in long DNA molecules is to cleave both strands of the DNA backbone at multiple sites, such as with a site-specific endonuclease which generates short single stranded overhangs at the cleavage site. Of the resulting segments, some are expected to contain mismatches. These can be removed by the action and subsequent removal of a mismatch binding protein, as described in FIG. 18. The remaining pool of segments can be re-ligated into full length sequences. As with the approach of FIG. 23, this approach includes several advantages including: 1) removal of an entire full length DNA duplex is not required to remove an error; 2) global dissociation and re-annealing of DNA duplexes is not necessary; 3) error-free DNA molecules can be constructed from a starting pool in which no one member is an error-free DNA molecule.

If the most common type of restriction endonuclease were employed for this approach, all DNA cleavage sites would result in identical overhangs. Thus the segments would associate and ligate in random order. However, use of a site-specific “outside cutter” endonuclease (such as HgaI, FokI, or BspMI) produces cleavage sites adjacent to (non-overlapping) the DNA recognition site. Thus each overhang would have sequence specific to that part of the DNA, distinct from that of the other sites. The re-association of these specifically complementary cohesive ends will then cause the segments to come together in the proper order. The cohesive ends generated can be up to five bases in length, allowing for up to 4⁵=1024 different combinations. Conceivably this many distinct restriction sites could be employed, though the need to avoid near matches between cohesive ends could lower this number.

The necessary restriction sites can be specifically included in the design of the sequence, or the random distribution of restriction sites within a desired sequence can be utilized (the recognition sequence of each endonuclease allows prediction of the typical distribution of fragments produced). Also, the target sequence can be analyzed for which choice of endonuclease produces the most ideal set of fragments.

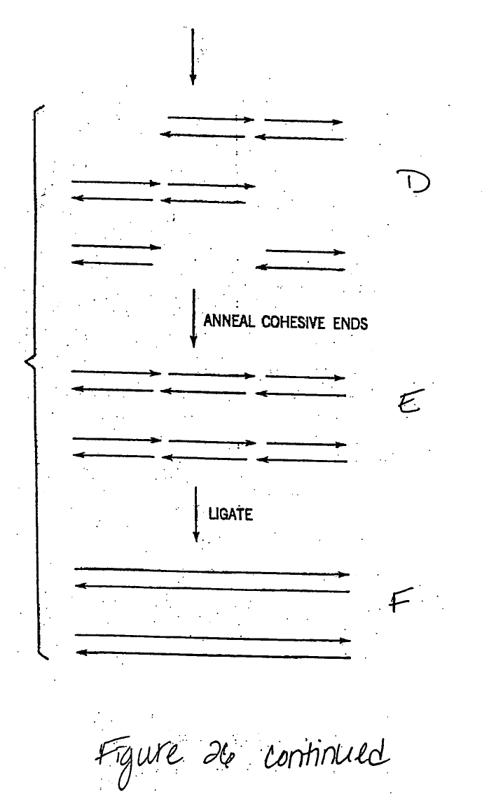

FIG. 26 shows an example of semi-selective removal of mismatch-containing segments. For example, FIG. 26A illustrates three DNA duplexes, each containing one error leading to a mismatch. The DNA is cut with a site-specific endonuclease, leaving double-stranded fragments with cohesive ends complementary to the adjacent segment (FIG. 26B). A MMBP is then applied, which binds to each fragment containing a mismatch (FIG. 26C). Fragments bound to MMBP are removed from the pool, as described in FIG. 18 (FIG. 26D). The cohesive ends of each fragment allow each DNA duplex to associate with the correct sequence-specific neighbor fragment (FIG. 26E). A ligase (such T4 DNA ligase) is employed to join the cohesive ends, producing full length DNA sequences (FIG. 26F). These DNA sequences can be error-free in spite of the fact that none of the original DNA duplexes was error-free. Incomplete ligation may leave some sequences which are less than full-length, which can be purified away on the basis of size.

The above approaches provide a major advantage over one of the conventional methods of removing errors, which employs sequencing first to find an error, and then relies on choosing specific error-free subsequences to “cut and paste” with endonuclease and ligase. In this embodiment, no sequencing or user choice is required in order to remove errors.

When complementary DNA strands are synthesized and allowed to anneal, both strands may contain errors, but the chance of errors occurring at the same base position in both sequences is extremely small, as discussed above. The above methods are useful for eliminating the majority case of uncorrelated errors which can be detected as DNA mismatches. In the rare case of complementary errors at identical positions on both strands (undetectable by the mismatch binding proteins), a subsequent cycle of duplex dissociation and random re-annealing with a different complementary strand (with a different distribution of error positions) remedies the problem. But in some applications it is desirable to not melt and re-anneal the DNA duplexes, such as in the case of genomic-length DNA strands. In such an embodiment, correlated errors may be removed using a different method. For example, though the initial population of correlated errors is expected to be low, amplification or other replication of the DNA sequences in a pool will ensure that each error is copied to produce a perfectly complementary strand which contains the complementary error. This approach does not require global dissociation and re-annealing of the DNA strands. Essentially, various forms of DNA damage and recombination are employed to allow single-stranded portions of the long DNA duplex to re-assort into different duplexes.

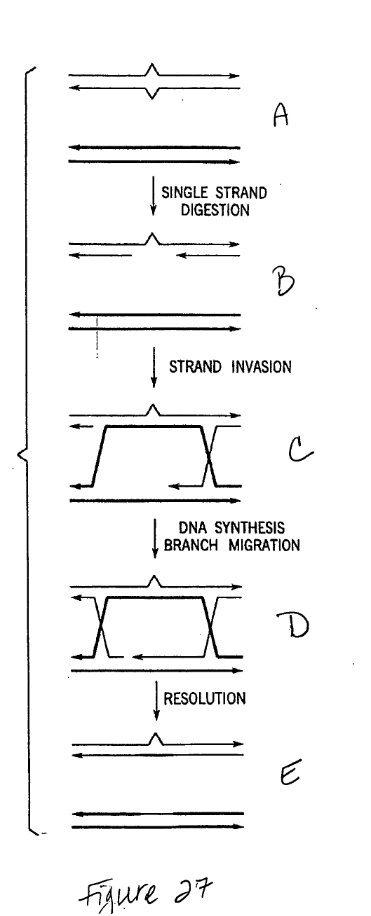

FIG. 27 shows a procedure for reducing correlated errors in synthesized DNA. FIG. 27A shows two DNA duplexes identical except for a single error in one strand. Non-specific nucleases may be used to generate short single-stranded gaps in random locations in the DNA duplexes in the pool (FIG. 27B). Shown here is the result of one of these gaps generated at the site of one of the correlated locations. Recombination-specific proteins such as RecA and RuvB are employed to mediate the formation of a four-stranded Holliday junction (FIG. 27C). DNA polymerase is employed to fill in the gap shown in the lower portion of the complex (FIG. 27D). Action of other recombination and/or repair proteins such as RuvC is employed to cleave the Holliday junction, resulting in two new DNA duplexes, containing some sequences which are hybrids of their progenitors (FIG. 27E). In the example shown, one of the error-containing regions has been eliminated. However, since the cutting, rearrangement, and replacement of strands employed in this method is intended to be random, it is expected that the total number of errors in the sequence will actually not change, simply that errors will be reasserted to different strands. Thus, pairs of errors correlated in one duplex will be reshuffled into separate duplexes, each with a single error. This random reassortment of strands will yield new duplexes containing mismatches which can be repaired using the mismatch repair proteins detailed above. Unique to this embodiment is the use of recombination to separate the correlated errors into different DNA duplexes.

8. Sequencing/In Vivo Selection

In certain embodiments, it may be desirable to evaluate successful assembly of a subassembly and/or synthetic polynucleotide construct by DNA sequencing, hybridization-based diagnostic methods, molecular biology techniques, such as restriction digest, selection marker assays, functional selection in vivo, or other suitable methods. For example, functional selection may be carried out by introducing a polynucleotide construct into a cell and assaying for expression of one or polynucleotides on the construct. Successful assemblies may be determined by assaying for a detectable marker, a selectable marker, a polypeptide of a given size (e.g., by size exclusion chromatography, gel electrophoresis, etc.), or by assaying for an enzymatic function of one or more polypeptides encoded by the polynucleotide construct. DNA manipulations and enzyme treatments are carried out in accordance with established protocols in the art and manufacturers' recommended procedures. Suitable techniques have been described in Sambrook et al. (2nd ed.), Cold Spring Harbor Laboratory, Cold Spring Harbor (1982, 1989); Methods in Enzymol. (Vols. 68, 100, 101, 118, and 152-155) (1979, 1983, 1986 and 1987); and DNA Cloning, D. M. Clover, Ed., IRL Press, Oxford (1985).

In certain embodiments, the polynucleotide constructs may be introduced into an expression vector and transfected into a host cell. The host cell may be any prokaryotic or eukaryotic cell. For example, a polypeptide of the invention may be expressed in bacterial cells, such as E. coli, insect cells (baculovirus), yeast, plant, or mammalian cells. The host cell may be supplemented with tRNA molecules not typically found in the host so as to optimize expression of the polypeptide. Ligating the polynucleotide construct into an expression vector, and transforming or transfecting into hosts, either eukaryotic (yeast, avian, insect or mammalian) or prokaryotic (bacterial cells), are standard procedures. Examples of expression vectors suitable for expression in prokaryotic cells such as E. coli include, for example, plasmids of the types: pBR322-derived plasmids, pEMBL-derived plasmids, pEX-derived plasmids, pBTac-derived plasmids and pUC-derived plasmids; expression vectors suitable for expression in yeast include, for example, YEP24, YIP5, YEP51, YEP52, pYES2, and YRP17; and expression vectors suitable for expression in mammalian cells include, for example, pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors.

9. Exemplary Uses

The polynucleotide constructs that can be synthesized in accordance with the compositions and methods described herein are essentially unlimited in variety. The methods provided herein permit the researcher to develop nucleic acid (and corresponding polypeptide) sequences from first principles without being bound by the limitations of naturally occurring sequences, site directed mutagenesis, or random mutagenesis techniques. Additionally, the methods permit the construction of very large, even genome sized, nucleic acid constructs, with high fidelity.

In one embodiment, the methods disclosed herein permit the production of codon remapped nucleotide sequences. The term “codon remapping” refers to modifying the codon content of a nucleic acid sequence without modifying the sequence of the polypeptide encoded by the nucleic acid. In certain embodiments, the term is meant to encompass “codon optimization” wherein the codon content of the nucleic acid sequence is modified to enhance expression in a particular cell type. In other embodiments, the term is meant to encompass “codon normalization” wherein the codon content of two or more nucleic acid sequences are modified to minimize any possible differences in protein expression that may arise due to the differences in codon usage between the sequences. In still other embodiments, the term is meant to encompass modifying the codon content of a nucleic acid sequence as a means to control the level of expression of a protein (e.g., either increases or decrease the level of expression). Codon remapping may be achieved by replacing at least one codon in the “wild-type sequence” with a different codon encoding the same amino acid that is used at a higher or lower frequency in a given cell type.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, nucleic acid sequences can be tailored for optimal expression in a given organism based on codon optimization.

Given the large number of gene sequences available for a wide variety of animal, plant and microbial species, it is possible to calculate the relative frequencies of codon usage. Codon usage tables are readily available, for example, at the “Codon Usage Database” available at http://www.kazusa.orjp/codon/, and these tables can be adapted in a number of ways. See Nakamura, Y., et al. “Codon usage tabulated from the international DNA sequence databases: status for the year 2000” Nucl. Acids Res. 28:292 (2000). These tables use mRNA nomenclature, and so instead of thymine (T) which is found in DNA, the tables use uracil (U) which is found in RNA. The tables have been adapted so that frequencies are calculated for each amino acid, rather than for all 64 codons.

By utilizing these or similar tables, one of ordinary skill in the art can apply the frequencies to any given polypeptide sequence, and produce a nucleic acid fragment of a codon-remapped coding region which encodes the same polypeptide, but which uses codons more or less optimal for a given species.

Codon-remapped coding regions can be designed by various different methods. For example, codon optimization may be carried out using a method termed “uniform optimization” wherein a codon usage table is used to find the single most frequent codon used for any given amino acid, and that codon is used each time that particular amino acid appears in the polypeptide sequence. For example, in humans the most frequent leucine codon is CUG, which is used 41% of the time. Therefore, codon optimization may be carried out by assigning the codon CUG for all leucine residues in a given amino acid.

In another method, termed “full-optimization,” the actual frequencies of the codons are distributed randomly throughout the coding region. Thus, using this method for optimization, if a hypothetical polypeptide sequence had 100 leucine residues and was to be optimized for expression in human cells, about 7, or 7% of the leucine codons would be UUA, about 13, or 13% of the leucine codons would be UUG, about 13, or 13% of the leucine codons would be CUU, about 20, or 20% of the leucine codons would be CUC, about 7, or 7% of the leucine codons would be CUA, and about 41, or 41% of the leucine codons would be CUG. These frequencies would be distributed randomly throughout the leucine codons in the coding region encoding the hypothetical polypeptide. As will be understood by those of ordinary skill in the art, the distribution of codons in the sequence can vary significantly using this method, however, the sequence always encodes the same polypeptide. Such methods may be adapted similarly adapted for other codon remapping techniques, including codon normalization.

Randomly assigning codons at an optimized frequency to encode a given polypeptide sequence, can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the VectorNTI Suite, available from InforMax, Inc., Bethesda, Md., and the “backtranslate” function in the GCG--Wisconsin Package, available from Accelrys, Inc., San Diego, Calif. In addition, various resources are publicly available to codon-optimize coding region sequences. For example, the “backtranslation” function at http://www.entelechon.com/eng/backtranslation.html, the “backtranseq” function available at http://bioinfo.pbi.nrc.ca:-8090/EMBOSS/index.html. Constructing a rudimentary algorithm to assign codons based on a given frequency can also easily be accomplished with basic mathematical functions by one of ordinary skill in the art.

In another embodiment, the methods disclosed herein may be used to synthesize viral genomes for a variety of applications including, viral vaccines, viral vectors for gene therapy, etc. The viral sequences may be designed to provide desired characteristics such as, attenuated viruses for vaccines, virus with lower antigenic or infectious properties for gene therapy applications, etc. For example, attenuated viruses can be used as vaccines against a broad range of viruses and/or antigens, including but not limited to antigens of strain variants, different viruses or other infectious pathogens (e.g., bacteria, parasites, fungi), or tumor specific antigens. In another embodiment, the attenuated viruses, which inhibit viral replication and tumor formation, can be used for the prophylaxis or treatment of infection (viral or nonviral pathogens) or tumor formation or treatment of diseases for which IFN is of therapeutic benefit. Many methods may be used to introduce the live attenuated virus formulations to a human or animal subject to induce an immune or appropriate cytokine response. These include, but are not limited to, intranasal, intratrachial, oral, intradermal, intramuscular, intraperitoneal, intravenous and subcutaneous routes. In a preferred embodiment, the attenuated viruses of the present invention are formulated for delivery intranasally. Any type of viral genome may be synthesized in accordance with the methods disclosed herein, including, for example, variants of DNA viruses, e.g., vaccinia, adenoviruses, hepadna viruses, herpes viruses, poxviruses, and parvoviruses; and RNA viruses, including hepatitis C3 virus, retrovirus, and segmented and non-segmented RNA viruses.

In another embodiment, the methods disclosed herein may be used to produce viral vectors suitable for gene therapy. Gene therapy is an area that offers an attractive approach for the treatment of many diseases and disorders. Many diseases are the result of genetic abnormalities such as gene mutations or deletions, and thus the prospect of replacing a damaged or missing gene with a fully functional gene is provocative. Throughout the last decade, studies of oncogenes and tumor suppressor genes have revealed increasing amounts of evidence that cancer is a disease caused by multiple genetic changes (Chiao et al., 1990; Levine, 1990; Weinberg, 1991; Sugimara et al., 1992). Based on this concept of carcinogenesis, new strategies of therapy have evolved rapidly as alternatives to conventional therapies such as chemo- and radiotherapy (Renan, 1990; Lotze et al., 1992; Pardoll, 1992). One of these strategies is gene therapy, in which tumor suppressor genes, antisense oligonucleotides, and other related genes are used to suppress the growth of malignant cells.

Gene therapy has also been contemplated for transfer of other therapeutically important genes into cells to correct genetic defects. Such genetic defects include deficiencies of adenosine deaminase that result in severe combined immunodeficiency, human blood clotting factor IX in hemophilia B, the dystrophin gene in Duchenne muscular dystrophy, and the cystic fibrosis transmembrane receptor in cystic fibrosis. Gene transfer in these situations requires long term expression of the transgene, and the ability to transfer large DNA fragments, such as the dystrophin cDNA, which is about 14 kB in size.

High efficiency transduction of cells and the ability to administer multiple doses of a therapeutic gene are particularly important points in gene therapy. The ability to transfer a gene into a cell requires a method of transferring the new genetic material across the plasma membrane of the cell and subsequent expression of the gene product to produce an effect on the cell. There are several means to transfer genetic material into a cell, including direct injection, lipofection, transfection of a plasmid, or transduction by a viral vector. The natural ability of viruses to infect a cell and direct gene expression make viral vectors attractive as gene transfer vectors. Other desirable elements of gene transfer vectors include a high transduction efficiency, large capacity for genetic material, targeted gene delivery, tissue-specific gene expression, and the ability to minimize host immunologic responses against the vector.

Many viral vectors have not produced the in vivo results that many have hoped. Expression levels and duration of expression appear to be two problems. It is thought that one of the causes for these problems is the toxicity and immunogenicity of virus, especially and high dosage. One way of attaining this goal is to reduce or eliminate the expression of viral proteins in the host. The diminution of viral gene expression and viral replication is desirable for the development of viral vectors used for gene therapy, for attenuated live viral vaccines and for the transformation of cells in vitro for the purpose of protein production. A common approach to this endeavor in the adenoviral system has been to delete certain viral genes.

In yet another embodiment, the methods disclosed herein may be used to produce polynucleotides containing various modifications at specific predetermined locations. Modifications that may be introduced into the polynucleotide constructs include, for example, modified bases (e.g., methylated bases, etc.), modified ribose rings, modified nucleobases, modified phosphate groups, modified backbone residues (e.g., phosphorthioate, etc.), and the production of peptide nucleic acid molecules (PNAs). Such modified polynucleotides may be useful for a variety of application in the fields of DNA diagnostics, therapeutics in the form of antisense and antigene, and the basic research of molecular biology and biotechnology (U. Englisch and D. H. Gauss, Angew. Chem. Int. Ed. Engl. 1991, 30, 613-629; A. D. Mesmaeker et al. Curr. Opinion Struct. Biol. 1995, 5, 343-355; P. E. Nielsen, Curr. Opin. Biotech., 2001, 12, 16-20.). PNA is DNA analogue in which an N-(2-aminoethyl)glycine polyamide replaces the phosphate-ribose ring backbone, and methylene-carbonyl linker connects natural as well as unnatural nucleo-bases to central amine of N-(2-aminoethyl)glycine. Despite radical change to the natural structure, PNA is capable of sequence specific binding to DNA as well as RNA obeying the Watson-Crick base pairing rule. PNAs bind with higher affinity to complementary nucleic acids than their natural counterparts, partly due to the lack of negative charge on backbone, a consequently reduced charge-charge repulsion, and favorable geometrical factors (S. K. Kim et al., J. Am. Chem. Soc., 1993, 115, 6477-6481; B. Hyrup et al., J. Am. Chem. Soc., 1994, 116, 7964-7970; M. Egholm et al., Nature, 1993, 365, 566-568; K. L. Dueholm et al., New J. Chem., 1997, 21, 19-31; P. Wittung et al., J. Am. Chem. Soc., 1996, 118, 7049-7054; M. Leijon et al., Biochemistry, 1994, 9820-9825.). The thermal stability of the resulting PNA/DNA duplex is independent of the salt concentration in the hybridization solution (H. Orum et al., BioTechniques, 1995, 19, 472-480; S. Tomac et al., J. Am. Chem. Soc., 1996, 118, 554-5552). Additionally, PNAs can bind in either parallel or antiparallel fashion, with antiparallel mode being preferred (E. Uhlman et al., Angew. Chem. Int. Ed. Engl., 1996, 35, 2632-2635).

In yet another embodiment, the methods disclosed herein may be used to produce polynucleotide constructs useful for studying epigenetics. Epigenetics refers to any change of the DNA structure, the chromatin or of the RNA which does not involve modifications of the nucleotides comprising the DNA or RNA. These changes can lead to the tri-dimensional modifications in DNA or chromatin structure. Examples of changes include chemical modifications of the purines or the pyrimidines constituting the DNA. In eukaryotes, a well known epigenetic regulation motif is the 5′CpG′ dinucleotides which can be methylated or unmethylated and thereby regulates transcription of a gene. In prokaryotes, a known epigenetic regulation motif includes the sequence 5′GATC3′.

5-methylcytosine is the most frequent covalent base modification in the DNA of eukaryotic cells. It plays a role, for example, in the regulation of the transcription, in genetic imprinting, and in tumorigenesis. For example, aberrant DNA methylation within CpG islands is common in human malignancies leading to abrogation or overexpression of a broad spectrum of genes (Jones, P. A., DNA methylation errors and cancer, Cancer Res. 65:2463-2467, 1996). Abnormal methylation has also been shown to occur in CpG rich regulatory elements in intronic and coding parts of genes for certain tumors (Chan, M. F., et al., Relationship between transcription and DNA methylation, Curr. Top. Microbiol. Immunol. 249:75-86, 2000). Using restriction landmark genomic scanning, Costello and coworkers were able to show that methylation patterns are tumour-type specific (Costello, J. F. et al., Aberrant CpG-island methylation has non-random and tumor-type-specific patterns, Nature Genetics 24:132-138, 2000). Highly characteristic DNA methylation patterns could also be shown for breast cancer cell lines (Huang, T. H.-M. et al., Hum. Mol. Genet. 8:459-470, 1999). DNA methylation may directly switch off gene expression, for example, by preventing transcription factors from binding to promoters. Additionally, methylated DNA attracts methyl-binding domain (MBD) proteins which are associated with further enzymes called histone deacetylases (HDACs). HDACs function to chemically modify histones and change chromatin structure. Chromatin containing acetylated histones is open and accessible to transcription factors, and the genes are potentially active. Histone deacetylation causes the condensation of chromatin making it inaccessible to transcription factors and the genes are therefore silenced. Since epigenetic modification plays an important role in various diseases such as cancer, the methods provided herein will permit synthesis of polynucleotide constructs that will be useful in screening for therapeutics or developing novel therapeutic strategies for modulating epigenetic regulation such as, for example, the reversal of DNA methylation or the inhibition of histone deacylation. In particular, the methods disclosed herein will permit synthesis of large polynucleotide constructs that may contain methylated residues at desired locations that can be used to study, for example, chromatin condensation under various screening conditions.

10. Implementation Systems and Methods

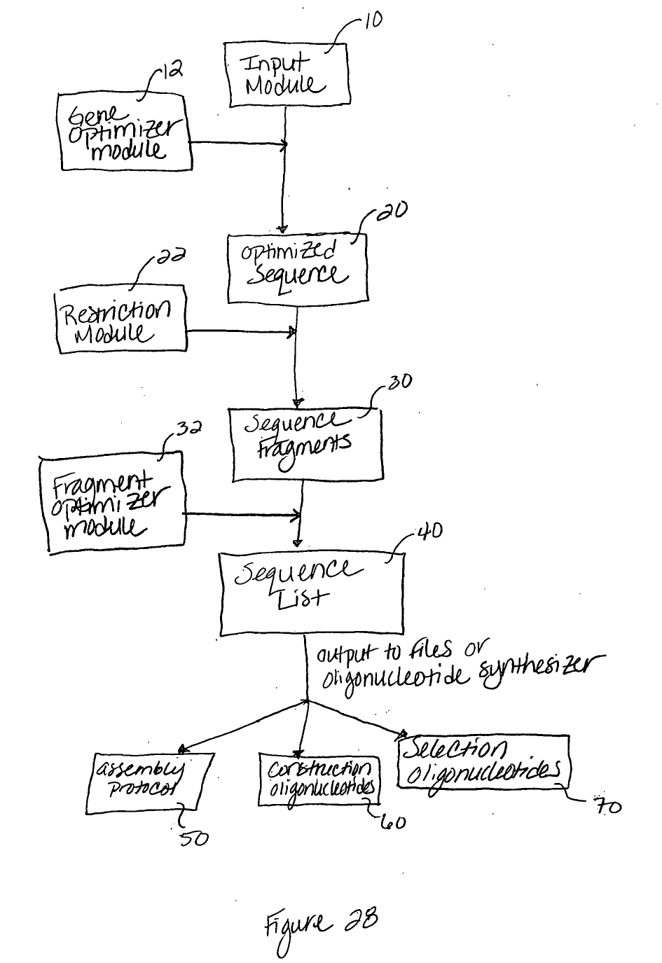

The disclosed methods and systems include methods and systems to design one or more sets of construction oligonucleotides, selection oligonucleotides, and/or to design an assembly strategy, for producing one or a plurality of polynucleotide constructs as described herein. FIG. 28 shows an illustrative block diagram for one embodiment of the disclosed methods and systems. As shown in FIG. 28, using the user input device 10 or another means, a user can input a sequence of a polynucleotide construct that is desired to be constructed and optionally other parameters. The user input device can be a processor-controlled device as provided herein, or can be provided with a user-interface that can allow a user or another to input information and/or data that can be used by the disclosed methods and systems. In various embodiments, the input sequence and/or parameters may be entered by the user or may be obtained from a database provided by the user, available over the internet, or available as part of the software program. Sequences and/or parameters obtained from a database may be provided by reference to a unique identifier rather than by input of the sequence and/or parameter itself. The user may input a single stranded or double stranded nucleic acid sequence (e.g., a DNA or RNA sequence) or may input a polypeptide sequence. When a polypeptide sequence is the input, the computer will reverse translate the sequence to produce one or more polynucleotide sequences that can encode the polypeptide sequence. The user may also input or reference a variety of parameters, including, for example: (i) the identity of an expression system (e.g., host cell, expression vector, regulatory sequences, etc.) that will be used to express a polynucleotide construct, (ii) whether or not the user wishes to conduct an error filtration process using selection oligonucleotides, (iii) whether the user wishes to construct a plurality of polynucleotide constructs in a single pool or multiple pools, (iii) whether the user wishes to amplify the construction oligonucleotides, selection oligonucleotides, subassemblies, and/or polynucleotide constructs, and/or (iv) information that classifies sections of an input polynucleotide sequence, such as, regulatory sequence, protein-coding sequence, RNA-coding sequence, and/or intergenic region. For the purposes of discussion with respect to the illustrative embodiments, reference is made to a single input sequence, although it can be understood that the methods and systems can be applied to one or more input sequences where such sequences can be in a single and/or multiple databases, and thus such discussion is merely for convenience and can be understood to encompass or otherwise embody multiple input sequences.

The user entered information can be provided to one or more servers, where such servers can be understood to be associated with one or more processor controlled devices as provided herein. Such servers can include instructions for accepting the user-provided information and for accessing processor-executable instructions as provided herein for providing and/or otherwise designing construction oligonucleotides, selection oligonucleotides, and/or an assembly strategy for preparing one or more polynucleotide constructs. The servers can have access to one or more databases which can include various types of information or analytical methods including, for example, methods for optimizing codon usage in a variety of host cells, methods for calculating melting temperature, methods for calculating sequence homology between two or more sequences, methods for determining secondary structure of nucleic acid sequences, methods for identifying restriction endonuclease binding and/or cleavage sites, methods for identifying binding and/or enzymatic sites for other proteins, such as, for example, mismatch binding proteins or mismatch repair proteins, etc., and/or methods for codon remapping sequences. In one embodiment, the user can request use of one or more of such analysis methods when designing construction and/or selection oligonucleotides by providing the aforementioned user-specified information at a user device, where such information can be transmitted to a server(s) via a wired or wireless connection using one or more intranets and/or the internet, where the servers can thereafter process the request by accessing the databases. Such database accessing can include querying the databases based on the user information. Upon completing the requested query and/or analysis, the servers can provide the user-device with outputs and/or results that can be provided to a memory, the device display, or other location.

Those of ordinary skill in the art will recognize that the illustrative system can be understood to be representative of a client-server paradigm, where the instructions on the user device for obtaining user information and requesting a comparison can be a client, and the servers can be a server in the client-server paradigm.

Accordingly, it can be understood that the user device instructions and instructions on the servers can be included in a single device, where such embodiment may also be considered within the client-server paradigm. The user device can access, via wired or wireless communications and using one or more intranets and/or the internet, the databases for querying, analyzing, and/or modifying sequences. Additionally, this embodiment can represent an embodiment that may not include a client-server paradigm.

With reference to FIG. 28, the gene optimizer module 12 takes the sequence and other parameters input by the user and determines an optimized polynucleotide sequence. The gene optimizer module will codon remap the sequence for optimized or normalized expression in a given host cell and/or to reduce secondary structure that may occur based on the input sequence. Preferably, the gene optimizer module modifies the polynucleotide sequence without modifying a polypeptide sequence encoded thereby. Alternatively, the gene optimizer module may minimize the effects of modification to the polypeptide sequence by optimizing the modifications, e.g., by controlling the location and/or identity of a modification (e.g., by only permitting modifications to a conserved residue). Various databases and algorithms for codon remapping are publicly available and are described further herein. The gene optimizer module results in an optimized sequence 20.

The optimized sequence 20 is then subjected to a restriction module 22 which divides the optimized sequence into fragments. The restriction module may divide the sequence into fragments based on the frequency and/or location of naturally occurring restriction sites in the optimized sequence. If the location of the naturally occurring restriction endonuclease sites are not optimal for the design of the construction oligonucleotides (e.g., the fragments are not of similar length, and/or have similar GC content), then the restriction module may codon remap the sequence to add or remove one or more restriction endonuclease sites from the sequence. Preferably the codon remapping will not, or will only minimally, affect the sequence of a polypeptide encoded by the polynucleotide. Alternatively, when using type IIS restriction endonuclease binding sites located in a flanking sequence, the restriction module may codon remap the sequence to remove any naturally occurring binding sites for the type IIS endonuclease from the sequence so as to prevent undesired cutting of the sequence. In another embodiment, the restriction module may divide the sequence into fragments of approximately the same size. The restriction module produces a set of sequence fragments that together define the input sequence 30.

The sequence fragments 30 are then subjected to a fragment optimizer module 32. The fragment optimizer module designs the sequences of the construction and/or selection oligonucleotides to be synthesized and assembled into a polynucleotide construct. The fragment optimizer module will design sequences of construction oligonucleotides that have overlapping sequences sufficient to permit assembly via the methods described herein. The fragment optimizer module will additionally design the sequences (e.g., selecting length, GC content, or by codon remapping) to produce a pool of construction oligonucleotides that has normalized melting temperature under a given set of hybridization conditions (which may be input by the user, selected from a parameters files, or determined by the software based on the design of the construction oligonucleotide sequences). If the user has indicated that error filtration using selective hybridization will be used, the fragment optimizer module may design one or more sets of selection oligonucleotides that may be used to purify the construction oligonucleotides as described further herein. The selection oligonucleotide sequences will be complementary to at least a portion of a construction oligonucleotide and may be designed as a set for optimal purification of a given set of construction oligonucleotides. Preferably a set of selection oligonucleotides will be optimized for hybridization to a set of construction oligonucleotides in a single reaction mixture (e.g., the melting temperatures of the pool of construction and selection oligonucleotides has been normalized). If the user has indicated that any of the oligonucleotides or polynucleotides will be amplified, the fragment optimizer module may add one or more primer hybridization sites onto the flanking regions of the construction and/or selection oligonucleotide sequences. These hybridization sites may be specified as an input or determined automatically by the algorithm based on the input sequences. Additionally, the fragment optimizer module may add restriction endonuclease sites into the flanking regions, e.g., a recognition sequence for a type IIS restriction endonuclease (such that the type IIS will remove the flanking sequence from the construction oligonucleotide sequences). Preferably, the fragment optimizer module will design a set of construction and/or selection oligonucleotides to contain primer hybridization sites and/or restriction endonuclease recognition sequences that are common to at least a portion of the construction and/or selection oligonucleotides. The sequence of the primers and/or restriction endonucleases to be used may be input by the user or may be designed by the fragment optimizer module. Additionally, the fragment optimizer module may utilize codon remapping to reduce homology between fragments. The fragment optimizer module 32 produces a sequence list 40 comprising the sequences of the construction and/or selection oligonucleotides to be synthesized and used to construct the input sequence. The fragment optimizer module may also specify an assembly protocol 50. The assembly protocol may be designed to be optimal with respect to process considerations such as cost, synthesis complexity, or product purity. The assembly protocol may specify subsets of the sequence list that should be assembled separately from the others and/or the order in which the subsets of the sequence list should be assembled. The sequence list 40 may then be output 60, 70 to a file which may be displayed to the user, stored in a computer readable medium (including a database), and/or printed out. The sequence list may also be output directly to an oligonucleotide synthesizer for preparation of the construction and/or selection oligonucleotides. The sequence list and the assembly protocol may also be output directly to a gene synthesizer for preparation of the entire, final sequence construct.

In various embodiments, software, or portions thereof, can be run in the RAM of general or special purpose computers or may be implemented in an application specific integrated circuit, digital signal processor, or other integrated circuit.

The methods and systems described herein are not limited to a particular hardware or software configuration, and may find applicability in many computing or processing environments. The methods and systems can be implemented in hardware or software, or a combination of hardware and software. The methods and systems can be implemented in one or more computer programs, where a computer program can be understood to include one or more processor executable instructions. The computer program(s) can execute on one or more programmable processors, and can be stored on one or more storage medium readable by the processor (including volatile and non-volatile memory and/or storage elements), one or more input devices, and/or one or more output devices. The processor thus can access one or more input devices to obtain input data, and can access one or more output devices to communicate output data. The input and/or output devices can include one or more of the following: Random Access Memory (RAM), Redundant Array of Independent Disks (RAID), floppy drive, CD, DVD, magnetic disk, internal hard drive, external hard drive, memory stick, or other storage device capable of being accessed by a processor as provided herein, where such aforementioned examples are not exhaustive, and are for illustration and not limitation.

The computer program(s) can be implemented using one or more high level procedural or object-oriented programming languages to communicate with a computer system; however, the program(s) can be implemented in assembly or machine language, if desired. The language can be compiled or interpreted.

As provided herein, the processor(s) can thus be embedded in one or more devices that can be operated independently or together in a networked environment, where the network can include, for example, a Local Area Network (LAN), wide area network (WAN), and/or can include an intranet and/or the internet and/or another network. The network(s) can be wired or wireless or a combination thereof and can use one or more communications protocols to facilitate communications between the different processors.

The processors can be configured for distributed processing and can utilize, in some embodiments, a client-server model as needed. Accordingly, the methods and systems can utilize multiple processors and/or processor devices, and the processor instructions can be divided amongst such single or multiple processor/devices.

The device(s) or computer systems that integrate with the processor(s) can include, for example, a personal computer(s), workstation (e.g., Sun, HP), personal digital assistant (PDA), handheld device such as cellular telephone, laptop, handheld, or another device capable of being integrated with a processor(s) that can operate as provided herein. Accordingly, the devices provided herein are not exhaustive and are provided for illustration and not limitation.

References to “a microprocessor” and “a processor”, or “the microprocessor” and “the processor,” can be understood to include one or more microprocessors that can communicate in a stand-alone and/or a distributed enviromnent(s), and can thus can be configured to communicate via wired or wireless communications with other processors, where such one or more processor can be configured to operate on one or more processor-controlled devices that can be similar or different devices. Use of such “microprocessor” or “processor” terminology can thus also be understood to include a central processing unit, an arithmetic logic unit, an application-specific integrated circuit (IC), and/or a task engine, with such examples provided for illustration and not limitation.

Furthermore, references to memory, unless otherwise specified, can include one or more processor-readable and accessible memory elements and/or components that can be internal to the processor-controlled device, external to the processor-controlled device, and/or can be accessed via a wired or wireless network using a variety of communications protocols, and unless otherwise specified, can be arranged to include a combination of external and internal memory devices, where such memory can be contiguous and/or partitioned based on the application. Accordingly, references to a database can be understood to include one or more memory associations, where such references can include commercially available database products (e.g., SQL, Informix, Oracle) and also proprietary databases, and may also include other structures for associating memory such as links, queues, graphs, trees, with such structures provided for illustration and not limitation.

References to a network, unless provided otherwise, can include one or more intranets and/or the internet. References herein to microprocessor instructions or microprocessor-executable instructions, in accordance with the above, can be understood to include programmable hardware.

Unless otherwise stated, use of the word “substantially” can be construed to include a precise relationship, condition, arrangement, orientation, and/or other characteristic, and deviations thereof as understood by one of ordinary skill in the art, to the extent that such deviations do not materially affect the disclosed methods and systems.

Elements, components, modules, and/or parts thereof that are described and/or otherwise portrayed through the figures to communicate with, be associated with, and/or be based on, something else, can be understood to so communicate, be associated with, and or be based on in a direct and/or indirect manner, uiless otherwise stipulated herein.

Certain illustrative embodiments of the systems and methods for carrying out the assembly methods described herein are described above. It will be understood by one of ordinary skill in the art that the systems and methods described herein can be adapted and modified to provide systems and methods for other suitable applications and that other additions and modifications can be made without departing from the scope of the systems and methods described herein.

Unless otherwise specified, the illustrated embodiments can be understood as providing exemplary features of varying detail of certain embodiments, and therefore, unless otherwise specified, features, components, modules, and/or aspects of the illustrations can be otherwise combined, separated, interchanged, and/or rearranged without departing from the disclosed systems or methods. Additionally, the shapes and sizes of components are also exemplary and unless otherwise specified, can be altered without affecting the scope of the disclosed and exemplary systems or methods of the present disclosure.

Although the methods and systems have been described relative to a specific embodiment thereof, they are not so limited. Obviously many modifications and variations may become apparent in light of the above teachings. Many additional changes in the details, materials, and arrangement of parts, herein described and illustrated, can be made by those skilled in the art. Accordingly, it will be understood that the following claims are not to be limited to the embodiments disclosed herein, can include practices otherwise than specifically described, and are to be interpreted as broadly as allowed under the law.

11. Automated System and Process for Custom-Designed Synthetic Polynucleotides

In one aspect, the present invention provides methods for interfacing computer technology with biological and chemical processing and synthesis equipment. In preferred embodiments, the present invention features methods for the computer to interface with equipment useful for biological and chemical processing and synthesis in a remote manner. Preferably, the methods of the present invention interface so as to run over a network or combination of networks such as the Internet, an internal network such as a company's own internal network, etc. thereby allowing the user to control the equipment remotely while maintaining a graphic display, updated in real time or near real time. Preferably, the methods of the present invention are used in conjunction with solid phase arrays that employ photolithographic or electrochemical methods for synthesis of chemical or biological materials.

In a second aspect, the present invention features a system for controlling and/or monitoring equipment for synthesizing or processing biological or chemical materials from a remote location. Such a system comprises a computer terminal remote from the equipment itself, software designed to monitor or control such equipment, and a communication means between the active part of such equipment and the computer terminal. Such a system preferably communicates between the computer terminal and the subject equipment via the internet or an internal intranet. Those skilled in the art readily understand that the software useful in such a system is highly specific depending upon the equipment itself and the parameter and conditions that need to be controlled or monitored to effect the desired processing or synthesis. As used herein, the term “remote” means not adjacent to. In effect, the term is used to denote that the computer terminal for effecting and monitoring the equipment may be located in the same vicinity as or in a completely location from the equipment. The present invention effectively allows the artisan to process or synthesize biological or chemical materials using appropriate equipment in a location that is removed from the equipment itself. Moreover, the present invention allows the artisan to control or monitor more than one or a plurality of pieces of equipment from such a remote location.

The present invention may be applied in, but is not limited to, the fields of chemical or biological synthesis such as the preparation of long, synthetic polynucleotides. The methods of the present invention are especially applicable to such equipment as DNA synthesizers, thermalcyclers, robotic instruments for controlled delivery of samples, etc. Such instruments may be controlled remotely according to the methods of the present invention thereby providing a graphic readout on progress and current status and controllable over a network.

The present invention provides a process for a manufacturer to obtain customer orders for custom-designed synthetic polynucleotides in an automated manner, comprising obtaining one or more desired sequence(s) from the customer, wherein the sequence(s) are single stranded or double stranded polynucleotide sequences (e.g., DNA or RNA) or polypeptide sequences; designing a set of construction oligonucleotides and/or selection oligonucleotides for production of the synthetic polynucleotides; designing a strategy for polynucleotide assembly that may involve, for example, rounds of amplification, error reduction, hierarchical assembly, etc.; synthesizing the set of construction and/or selection oligonucleotides; and assembling the construction oligonucleotides into the polynucleotide construct using the assembly strategy.

Preferably, the step of designing the set of construction and/or selection oligonucleotides comprises developing binding regions between complementary oligonucleotides according to consistent reaction conditions, wherein the reaction conditions include temperature, buffer conditions (including for example, pH and salt concentration), etc.

Preferably, the construction and/or selection oligonucleotides may be synthesized on a solid support using any of a variety of methods for array synthesis such as, for example, in situ synthesis of oligonucleotides by spotting (e.g., inkjet methods), in situ synthesis of oligonucleotides by photolithography methods, electrochemical-based pH changes in situ synthesis of oligonucleotides, photochemical-based pH changes for in situ synthesis of oligonucleotides, maskless array synthesis methods, and combinations thereof.

The present invention further provides a system for a manufacturer to obtain customer orders for custom-designed synthetic polynucleotide and/or polypeptide sequences comprising a network-based receiving station for a manufacturer to receive desired synthetic polynucleotide and/or polypeptide sequences from the customer; a software means for designing a set of construction and/or selection oligonucleotides and/or designing an assembly strategy; and a manufacturing system for synthesizing the construction oligonucleotides and assembling the polynucleotide constructs. Preferably, the software means designs the construction oligonucleotides and/or selection oligonucleotides to provide substantially uniform melting temperatures, G/C vs. AT content, pH, environment, stringency conditions, or other conditions for consistent hybridization of oligonucleotide sequence(s). The software means may further design universal tags (including universal primers) common to at least a portion of the construction and/or selection oligonucleotides. For example, the software may design primer binding sites and/or restriction endonuclease binding and cleavage sites to be added to flanking regions of the construction and/or selection oligonucleotides. The software may additional design primer sequences, select a restriction endonuclease, determine appropriate reaction conditions for PCR and/or enzyme digestion, etc. When assembling a plurality of constructs, particularly constructs having regions on internal homology, the software may additionally design an assembly strategy that permits assembly of a plurality of constructs in a single pool. Alternatively, the software may design a hierarchical assembly strategy for production of the polynucleotide constructs. In certain embodiments, the sequences for the set of construction and/or selection polynucleotides and/or the instructions for the assembly strategy may be retained within a storage device at the manufacturer. In certain embodiments, the customer may provide their own sequences for synthesis. Alternatively, the customers may be able to select a synthetic polynucleotide sequence for synthesis from a database of synthetic polynucleotide and/or polypeptide sequences.

Preferably, the design of construction and/or selection oligonucleotides comprises developing complementary binding regions between various construction and/or selection oligonucleotides according to consistent reaction conditions, wherein the reaction conditions include temperature, pH, stringency, ionic strength, hydrophilic or hydrophobic environment, nucleotide content, oligonucleotide length, and combinations thereof wherein a software program having melting temperature, stringency and proton (pH) chemistry algorithms is employed. In an exemplary embodiment, the software program may also optimize sequences by codon remapping to reduce regions of homology between two or more sequences, to remove and/or add one or more restriction endonuclease recognition and/or cleavage sites, to optimize or normalize expression in a particular expression system, and/or to reduce regions of secondary structure.

For example, a system may be employed whereby a researcher/customer designs a synthetic polynucleotide sequence using a computer at the remote (customer/researcher) location. The customer requests are transmitted to another computer that accesses at least one database to complete design of construction oligonucleotides and/or selection oligonucleotides and/or an assembly strategy. Alternatively, the customer's remote computer may access at least one database during the design stage and send a complete design of construction oligonucleotides and/or selection oligonucleotides and/or an assembly strategy to the local server. The local computer sends the complete design of construction oligonucleotides and/or selection oligonucleotides and/or an assembly strategy to an automated array fabrication unit, which constructs an array according to the design set of construction and/or selection oligonucleotides. The oligonucleotides are then assembled into the polynucleotide construct according to the assembly strategy. Preferably, the assembly takes places in a high-throughput and/or automated fashion using computer directed instruments such as thermocyclers and/or robotic systems for sample mixing, etc.

The present invention further provides a user interface that a user can employ at a location that might be different from or remote from the site of manufacture of the array. This interface can provide the user with a way to specify the polynucleotide sequence to be synthesized, the degree of errors that will be tolerated for the desired application,.the amount of polynucleotide that will be required, etc. The interface is deployed as a custom application that runs on a computer at the user's location, an applet that runs over a network, such as the Internet (such as with Java or Active X), a downloadable application, HTML forms, DHTML pages, XML forms, or any other technology that provides for interaction with the user and communication of data.

In a preferred embodiment, the synthesis of the polynucleotide construct is automated. A device (again, possibly at a site remote from the user) can take a specification for the polynucleotide sequence to be synthesized and produce the polynucleotide construct from that specification.

From a user's point of view, the user will first specify which polynucleotide sequences he or she is interested in synthesizing. Second, a server or servers (possibly with human intervention or help) will take the specification and design a set of construction oligonucleotides and/or selection oligonucleotides and/or an assembly strategy. Third, the server will send the oligonucleotide set design and assembly strategy to a DNA-array synthesizer that will synthesize the oligonucleotides. Fourth, the oligonucleotides will be cleaved from the array and subjected to assembly in an automated or semi-automated fashion. The assembly strategy may involve multiple rounds of amplification, error reduction and/or assembly. Fifth, after a polynucleotide construct is made that passes quality-control checks, the polynucleotide construct is shipped to the user.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Equivalents

The present invention provides among other things synthetic polynucleotide constructs and methods for producing synthetic polynucleotide constructs. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: Prodromou and Pearl (1992) Protein Eng. 5:827; Dillon, P. J. and Rosen, C. A. (1993) In White, B. A. (ed.), PCR Protocols: Current Methods and Applications. Humana Press, Totowa, N.J., Vol. 15, pp. 263-267; Sardana et al. (1996) Plant Cell Rep. 15: 677; Stermner (1994) Proc. Natl. Acad. Sci. U.S.A. 91: 10747; Ho et al. (1989) Gene 77: 51; PCT Publication Nos. WO 99/42813; WO 99/41007; WO 96/33207; WO 04/039953; WO 04/031399; WO 04/031351; WO 04/029586; WO 03/100012; WO 03/085094; WO 03/072832; WO 03/066212; WO 03/065038; WO 03/064699; WO 03/064027; WO 03/064026; WO 03/054,232; WO 03/046223; WO 03/040410; WO 02/44425; WO 02/095073; WO 02/095073; WO 02/081490; WO 02/072791; WO 02/04680; WO 02/04597; WO 02/02227; WO 01/34847; WO 01/34847; U.S. Patent Publication Nos. 2004/0132029; 2004/0101444; 2003/0118486; 2003/0068643; 2004/0132029; 2004/0132029; 2004/0126757; 2004/0110212; 2004/0110211; 2004/0101949; 2004/0101894; 2004/0014083; 2004/0009520; 2003/0186226; 2003/0087298; 2003/0068633; 2002/0081582; U.S. Pat. Nos. 6,670,127; 6,664,112; 6,650,822; 6,600,031; 6,586,211; 6,566,495; 6,521,427; 6,489,146; 6,480,324; 6,444,175; 6,426,184; 6,406,847; 6,375,903; 6,372,434; 6,365,355; 6,346,413; 6,346,399; 6,315,958; 6,291,242; 6,287,861; 6,287,825; 6,284,463; 6,271,957; 6,165,793; 6,150,102; 6,054,270; 6,027,877; 5,953,469; 5,928,905; 5,922,539; 5,916,794; 5,861,482; 5,858,754; 5,834,252; 5,750,335; 5,744,305; 5,702,894; 5,700,637; 5,679,522; 5,605,793; 5,556,750; 5,459,039; 5,445,934; 5,436,327; 5,436,150; 5,424,186; 5,405,783; 5,356,802; 4,999,294; 4,965,188; 4,800,159; 4,683,202; and 4,683,195.

1. A method for assembling a long polynucleotide construct having a predefined sequence, comprising:
a) providing a pool of construction oligonucleotides;
b) conducting i), ii), or i) and ii) in either order or simultaneously:
i) amplifying said construction oligonucleotides; and
ii) subjecting said construction oligonucleotides to an error reduction process; and
c) exposing said pool of construction oligonucleotides to hybridization conditions and one or more of the following conditions: (i) ligation, (ii) chain extension, or (iii) chain extension and ligation conditions, thereby forming a plurality of copies of at least one double stranded subassembly construct that is longer than said construction oligonucleotides;
d) conducting i), ii), or i) and ii) in either order or simultaneously:
i) amplifying said subassembly construct; and
ii) subjecting said subassembly construct to an error reduction process; and
e) incubating two or more subassembly constructs under hybridization conditions and one or more of the following conditions: (i) ligation, (ii) chain extension, or (iii) chain extension and ligation conditions, thereby forming a plurality of copies of a long polynucleotide construct.

2. The method of claim 1, wherein the construction oligonucleotides are subjected to an error filtration process.

3. The method of claim 2, wherein the error filtration process comprises:
a) contacting the pool of construction oligonucleotides with a pool of selection oligonucleotides under hybridization conditions to form duplexes, wherein the selection oligonucleotides comprise sequences that are complementary to at least portions of the construction oligonucleotides, and wherein at least a portion of the duplexes are stable duplexes comprising a copy of a construction oligonucleotide and a copy of a selection oligonucleotide that do not contain a mismatch in the complementary region and a portion of the duplexes are unstable duplexes comprising a copy of a construction oligonucleotide and a copy of a selection oligonucleotide that contain one or more mismatches in the complementary region;
b) removing copies of the construction oligonucleotides that have formed unstable duplexes; and
c) denaturing the remaining duplexes thereby forming a purified pool of construction oligonucleotides; and
d) incubating the purified pool of construction oligonucleotides under hybridization conditions and at least one of the following conditions: (i) ligation conditions, (ii) chain extension conditions, or (ii) chain extension and ligation conditions, thereby forming at least one subassembly construct.

4. The method of claim 3, wherein the selection oligonucleotides are immobilized on a solid support.

5. The method of claim 3, wherein the selection oligonucleotides comprise a biotin group at one terminus.

6. The method of claim 3, wherein copies of the construction oligonucleotides that have formed unstable duplexes are removed from the pool by controlling the stringency of hybridization or wash conditions, or both.

7. The method of claim 4, wherein the solid support is a column or beads.

8. The method of claim 3, further comprising amplifying the construction oligonucleotides prior to forming the subassembly construct.

9. The method of claim 3, further comprising repeating a)-c) at least one time prior to forming the subassembly construct.

10. The method of claim 1, wherein the construction oligonucleotides or the subassembly constructs are subjected to an error filtration process, comprising:
a) incubating the construction oligonucleotides or the subassembly construct with at least one agent that binds to a mismatch; and
b) removing copies of the construction oligonucleotides or the subassembly construct that bound to the agent.

11. The method of claim 10, wherein the agent is a mismatch binding protein.

12. The method of claim 11, wherein the mismatch binding protein is one or more of the following: Fok I, T7 endonuclease, mutH, mutL, mutM, mutS, mutY, dam, thymidine DNA glycosylase (TDG), uracil DNA glycosylase, AlkA, MLH1, MSH2, MSH3, MSH6, Exonuclease I, T4 endonuclease V, Exonuclease V, RecJ exonuclease, FEN1 (RAD27), dnaQ (mutD), or polC (dnaE).

13. The method of claim 12, wherein the mismatch binding protein is mutS.

14. The method of claim 13, wherein the construction oligonucleotides or the subassembly construct are incubated with mutS in the presence of ATP.

15. The method of claim 14, wherein the ATP is present in an amount sufficient to increase the affinity of mutS for a duplex containing a mismatch to less than about 10 nanomolar.

16. The method of claim 12, wherein the mismatch binding protein is a DNA glycosylase.

17. The method of claim 12, wherein the mismatch binding protein is TDG.

18. The method of claim 10, further comprising amplification of the copies of the construction oligonucleotides or the subassembly construct that did not bind to the agent.

19. The method of claim 18, further comprising repeating a) and b) at least one time.

20. The method of claim 18, wherein copies of the construction oligonucleotides or the subassembly construct that bound to the agent are removed by gel filtration.

21. The method of claim 18, wherein the agent is immobilized on a substrate.

22. The method of claim 18, wherein the substrate is a column or beads.

23. The method of claim 18, further comprising cross-linking the agent to the construction oligonucleotides or the subassembly construct.

24. The method of claim 1, wherein the construction oligonucleotides or the subassembly construct are subjected to an error neutralization process, comprising:
a) incubating the construction oligonucleotides or the subassembly construct with an agent that binds to a mismatch; and
b) crosslinking the agent to the copies of the construction oligonucleotides or the subassembly construct that contain a mismatch; and
c) amplifying the construction oligonucleotides or the subassembly construct, wherein copies of the construction oligonucleotides or the subassembly construct that are crosslinked to the agent are not amplified exponentially and are diluted out.

25. The method of claim 1, wherein the subassembly construct is subjected to an error correction process.

26. The method of claim 25, wherein the error correction process comprises:
a) incubating a plurality of copies of the subassembly construct with an agent that cleaves the subassembly construct to create a double stranded break and remove the mismatch;
b) melting and reannealing the plurality of copies of the subassembly construct;
c) incubating the plurality of copies of the subassembly construct under hybridization and chain extension conditions, wherein strands of the subassembly construct that were cleaved by the agent can hybridize to overlapping, complementary strands and serve as primers for chain extension.

27. The method of claim 26, wherein the agent is a fusion protein comprising a mismatch binding protein, or a functional fragment thereof, and a nuclease, or a functional fragment thereof.

28. The method of claim 26, wherein the agent is Fok I, T7 endonuclease I or T4 endonuclease.

29. The method of claim 25, wherein the error correction process comprises:
a) contacting a plurality of copies of the subassembly construct with a methylation agent;
b) contacting the plurality of copies of the subassembly construct with a site-specific demethylation agent;
c) denaturing the plurality of copies of the subassembly construct;
d) renaturing the plurality of copies of the subassembly construct thereby forming a plurality of copies of the double stranded subassembly construct at least a portion of which comprise at least one hemimethylated region; and
e) contacting the plurality of copies of the subassembly construct with a mismatch repair system.

30. The method of claim 29, wherein the site-specific demethylation agent is a fusion protein comprising a mismatch binding protein, or a functional fragment thereof, and a demethylase, or a functional fragment thereof.

31. The method of claim 25, wherein the error correction process comprises:
a) incubating a plurality of copies of the subassembly construct with an agent that cleaves the subassembly construct to create a double stranded break and remove the mismatch;
b) contacting the plurality of copies of the subassembly construct with an agent that promotes formation of Holliday junctions;
c) incubating the plurality of copies of the subassembly construct under conditions that promote chain extension; and
d) contacting the plurality of copies of the subassembly construct with an agent that promotes resolution of the Holliday junctions.

32. The method of claim 1, further comprising subjecting the long polynucleotide construct to an error reduction process.

33. The method of claim 32, wherein the error reduction process comprises:
a) contacting the plurality of copies of the long polynucleotide construct with an agent that causes site-specific double stranded breaks and cohesive ends thereby producing fragments;
b) contacting the fragments with an agent that binds to a mismatch;
c) removing fragments that bound to the agent; and
d) incubating the fragments under conditions that promote hybridization and ligation of the cohesive ends, thereby reforming the long polynucleotide construct.

34. The method of claim 33, wherein the agent that causes site-specific double stranded breaks is FokI, T7 endonuclease I, or T4 endonuclease.

35. The method of claim 32, wherein the error reduction process comprises:
a) contacting the plurality of copies of the long polynucleotide construct with an agent that causes non-specific single-stranded breaks;
b) contacting the plurality of copies of the long polynucleotide construct with an agent that promotes formation of Holliday junctions;
c) incubating the plurality of copies of the long polynucleotide construct under conditions that promote chain extension; and
d) contacting the plurality of copies of the long polynucleotide construct with an agent that promotes resolution of the Holliday junctions

36. The method of claim 32, wherein the error reduction process comprises:
a) contacting the plurality of copies of the long polynucleotide construct with an agent that cause non-specific double stranded breaks thereby forming double stranded fragments;
b) denaturing the fragments thereby forming single stranded fragments;
c) incubating the fragments under conditions which promote hybridization between complementary overlapping single stranded fragments;
d) contacting the fragments with an agent that binds to a mismatch;
e) removing fragments that bound to the agent; and
f) incubating fragments under hybridization conditions and at least one of the following conditions: (i) ligation, (ii) chain extension, or (iii) chain extension and ligation conditions, to reassemble the long polynucleotide construct.

37. The method of claim 1, wherein said pool forms a plurality of double stranded subassembly constructs.

38. The method of claim 1, wherein the two or more subassembly constructs are assembled in separate reactions.

39. The method of claim 1, wherein the two or more subassembly constructs are assembled in the same reaction.

40. The method of claim 1, wherein a plurality of subassembly constructs are incubated under hybridization conditions and at least one of the following conditions: (i) ligation conditions, (ii) chain extension conditions, (iii) chain extension and ligation conditions, thereby forming a plurality of long polynucleotide constructs.

41. The method of claim 1, further comprising a round of denaturation and renaturation prior to conducting an error reduction process.

42. The method of claim 1, wherein the construction oligonucleotides are from about 20 to about 150 nucleotides in length.

43. The method of claim 1, wherein the subassembly construct is at least about 200 to about 750 nucleotides in length.

44. The method of claim 1, wherein the subassembly construct is at least about 5 times as long as the construction oligonucleotides.

45. The method of claim 1, wherein the polynucleotide construct is at least about 5 times as long as the subassembly construct.

46. The method of claim 1, wherein the polynucleotide construct is at least about 1 kilobase in length.

47. The method of claim 46, wherein the polynucleotide construct is at least about 10 kilobases in length.

48. The method of claim 47, wherein the polynucleotide construct is at least about 100 kilobases in length.

49. The method of claim 1, wherein less than about 99% of the copies of the subassembly or polynucleotide constructs have a sequence error.

50. The method of claim 49, wherein less than about 95% of the copies of the subassembly or polynucleotide constructs have a sequence error.

51. The method of claim 50, wherein less than about 90% of the copies of the subassembly or polynucleotide constructs have a sequence error.

52. The method of claim 51, wherein less than about 50% of the copies of the subassembly or polynucleotide constructs have a sequence error.

53. The method of claim 1, wherein said oligonucleotides are synthesized on a solid support.

54. The method of claim 53, wherein the oligonucleotides are attached to the solid support by a cleavable linker.

55. The method of claim 54, wherein the linker is a chemically cleavable linker, a thermally cleavable linker, an enzymatically cleavable linker, or a photocleavable linker.

56. The method of claim 53, wherein the synthesis uses light triggered reactions at discrete location on said support.

57. The method of claim 56, wherein the light is directed to discrete locations using masks.

58. The method of claim 57, wherein the light is directed to discrete locations using light directing maskless optics.

59. The method of claim 53, wherein the oligonucleotides are severed from the solid support prior to amplification.

60. The method of claim 1, wherein said oligonucleotides comprise at least one pair of primer hybridization sites flanking at least a portion of said oligonucleotides and common to at least a subset of said oligonucleotides.

61. The method of claim 60, wherein all of the oligonucleotides comprise at least one pair of primer hybridization sites in common.

62. The method of claim 60, wherein said oligonucleotides comprise cleavage sites between at least a portion of the primer hybridization sites and the oligonucleotides.

63. The method of claim 62, wherein the cleavage site is a restriction endonuclease site.

64. The method of claim 63, wherein the restriction endonuclease is a type IIS endonuclease.

65. The method of claim 1, wherein said subassembly constructs comprise at least one pair of primer hybridization sites flanking at least a portion of said subassembly constructs and common to at least a subset of said subassembly constructs.

66. The method of claim 65, wherein the subassembly constructs comprise a cleavage site between at least one of the primer hybridization sites and the subassembly constructs.

67. The method of claim 1, wherein the amplification of the construction oligonucleotides or the subassembly constructs uses at least one primer containing at least one uracil residue.

68. The method of claim 67, wherein the uracil residue is located at the junction between the primer hybridization site and the construction oligonucleotides or the subassembly constructs.

69. The method of claim 67, wherein the primer hybridization site is removed using uracil DNA glycosylase and an AP endonuclease.

70. The method of claim 1, wherein at least two subassembly constructs are formed in the same reaction mixture.

71. The method of claim 70, wherein at least four subassembly constructs are formed in the same reaction mixture.

72. The method of claim 71, wherein at least ten subassembly constructs are formed in the same reaction mixture.

73. The method of claim 1, wherein at least two polynucleotide constructs are formed in the same reaction mixture.

74. The method of claim 73, wherein at least four polynucleotide constructs are formed in the same reaction mixture.

75. The method of claim 74, wherein at least ten polynucleotide constructs are formed in the same reaction mixture.

76. A method for preparing a long polynucleotide construct having a predefined sequence, comprising:
a) providing a pool of input oligonucleotides under hybridization conditions and at least one of the following conditions: (i) ligation conditions, (ii) chain extension conditions, or (iii) chain extension and ligation conditions, wherein said pool comprises a plurality of overlapping sequences, and wherein said pool forms at least one product polynucleotide that is longer than said oligonucleotides;
b) conducting i) and ii) in either order or simultaneously:
i) amplifying said product polynucleotides; and
ii) subjecting said product polynucleotides to an error reduction process; and
c) repeating steps a) and b) at least two times, wherein said product polynucleotides constitute the input oligonucleotides in the next cycle.

77. The method of claim 76, wherein the pool of input oligonucleotides comprises positive and negative strands that are complementary in the overlapping regions.

78. The method of claim 76, wherein the pool of input oligonucleotides is amplified prior to forming a polynucleotide product.

79. The method of claim 76, wherein the pool of input oligonucleotides is subjected to an error reduction process prior to forming a polynucleotide product.

80. The method of claim 79, wherein the error reduction process is error filtration using a pool of selection oligonucleotides.

81-162. (canceled)
